(19) United States
(12) Patent Application Publication
(10) Pub. No.: US 2026/0042848 A1
(43) Pub. Date: Feb. 12, 2026

(54) ANTI-AXL ANTIBODIES, ANTIGEN-BINDING FRAGMENTS THEREOF AND METHODS FOR MAKING AND USING THE SAME

(71) Applicant: BioLegend, Inc., San Diego, CA (US)

(72) Inventors: Takatoku OIDA, Tokyo (JP); Shuping YIN, San Diego, CA (US); Nicolas SCHRANTZ, San Diego, CA (US)

(21) Appl. No.: 19/099,222

(22) PCT Filed: Aug. 16, 2023
(86) PCT No.: PCT/US2023/072304

§ 371 Date: Jan. 28, 2025

Related U.S. Application Data
(60) Provisional application No. 63/399,044, filed on Aug. 18, 2022.

(51) Int. Cl. C07K 16/28; G01N 33/68

(52) U.S. Cl. CPC C07K 16/2863; G01N 33/6893; C07K 2317/76; G01N 2333/71

(57) ABSTRACT

Compositions and methods for making and using anti-AXL antibodies or antigen-binding fragments thereof, for example, monoclonal antibodies. AXL-binding antibody fragments, and derivatives are described, as are nucleic acids encoding such molecules, diagnostic reagents and kits that include anti-AXL antibodies or antigen-binding fragments thereof, and methods of making and using the same.

### CROSS REFERENCE TO RELATED APPLICATION

The present application claims priority to U.S. Provisional Application No. 63,399,044, filed Aug. 18, 2022, which is incorporated by reference herein in its entirety.

### FIELD

The technology relates in part to antibodies and antigen-binding fragments thereof that bind tyrosine kinase receptor AXL and its variants, particularly to monoclonal antibodies, antigen binding fragments, and antibody derivatives specifically reactive to AXL under physiological and/or in vitro conditions. Such antibodies and antigen-binding fragments thereof can be useful for laboratory/research purposes (e.g., flow cytometry), and may be used in the treatment and/or prevention of various diseases or disorders through the delivery of pharmaceutical or other compositions that contain such antibodies and antigen-binding fragments thereof.

### BACKGROUND

The tyrosine-protein kinase receptor UFO, also known as AXL, is a widely expressed glycoprotein in the TAM receptor tyrosine kinase family. The function of AXL has been implicated in leukocyte adhesion to Extracellular Matrix (ECM) proteins and diapedesis across the vascular endothelium and is capable of promoting metastatic diffusion and epithelial-mesenchymal transition. AXL has also been implicated as a host cell receptor for multiple viruses, including Marburg, Ebola and Lassa viruses and is a candidate receptor for the SARS-CoV2 virus However, currently available anti-AXL antibodies and associated methods are limited in their range of both in vitro and in vivo applications. Thus, there remains a need for reagents, devices and methods of detecting AXL, and diagnosing and/or treating AXL related diseases or disorders. Provided herein are embodiments that meet such needs.

### SUMMARY

Disclosed is an isolated antibody or antigen binding fragment thereof that specifically binds to AXL, wherein the isolated antibody comprises: a) a heavy chain variable region comprising: (i) a heavy chain complementarity determining region 1 (CDRH1) comprising a sequence set forth in SEQ ID NO:24 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:24; (ii) a heavy chain complementarity determining region 2 (CDRH2) comprising a sequence set forth in SEQ ID NO: 27 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:27; and, (iii) a heavy chain complementarity determining region 3 (CDRH3) comprising a sequence set forth in SEQ ID NO:31 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:31; and, b) a light chain variable region comprising: (i) a light chain complementarity determining region 1 (CDRL1) comprising a sequence set forth in SEQ ID NO:36 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:36; (ii) a light chain complementarity determining region 2 (CDRL2) comprising a sequence set forth in SEQ ID NO: 40 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:40; and, (iii) a light chain complementarity determining region 3 (CDRL3) comprising the sequence set forth in SEQ ID NO:45 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:45.

In an embodiment, the heavy chain variable region comprises at least 90% identity to the sequence set forth in SEQ ID NO:1. In some embodiments, the light chain variable region comprises at least 90% identity to a sequence set forth in SEQ ID NO:7. Accordingly, in some embodiments, the heavy chain variable region comprises at least 90% identity to a sequence set forth in SEQ ID NO:1, and the light chain variable region comprises at least 90% identity to a sequence set forth in SEQ ID NO:7.

In certain aspects, the isolated antibody or antigen binding fragment further comprises an Fc polypeptide having at least 90% identity to a sequence set forth in SEQ ID NO:63.

Also disclosed are a pharmaceutical composition comprising the isolated antibody or antigen binding fragment and a pharmaceutically acceptable carrier; a diagnostic reagent comprising the isolated antibody or antigen binding fragment thereof; and, kits comprising the isolated antibody or antigen binding fragment thereof, or the diagnostic reagent.

The disclosure also includes an isolated nucleic acid comprising a nucleotide sequence that encodes a heavy chain variable region comprising a sequence set forth in SEQ ID NO: 13 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:13; and, an isolated nucleic acid comprising a nucleotide sequence that encodes a light chain variable region comprising a sequence set forth in SEQ ID NO:19 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:19. Also disclosed is an expression vector comprising the aforementioned nucleic acids, and an isolated host cell comprising the aforementioned expression vector.

In another embodiment, disclosed is an isolated antibody or antigen binding fragment thereof that specifically binds to AXL, wherein the isolated antibody comprises: a) a heavy chain variable region comprising: (i) a heavy chain complementarity determining region 1 (CDRH1) comprising a sequence set forth in SEQ ID NO:25 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:25; (ii) a heavy chain complementarity determining region 2 (CDRH2) comprising a sequence set forth in SEQ ID NO: 28 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:28; and, (iii) a heavy chain complementarity determining region 3 (CDRH3) comprises a sequence set forth in SEQ ID NO:32 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:32; and, b) a light chain variable region comprising: (i) a light chain complementarity determining region 1 (CDRL1) a sequence set forth in SEQ ID NO:37 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:37; (ii) a light chain complementarity determining region 2 (CDRL2) comprising a sequence set forth in SEQ ID NO: 41 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:41; and, (iii) a light chain complementarity determining region 3 (CDRL3) comprising a sequence set forth in SEQ ID NO:46 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:46.

In certain such embodiments, the heavy chain variable region comprises at least 90% identity to the sequence set forth in SEQ ID NO:2. In certain such embodiments, the light chain variable region comprises at least 90% identity to the sequence set forth in SEQ ID NO:8. Accordingly, in certain such embodiments, the heavy chain variable region comprises at least 90% identity to a sequence set forth in SEQ ID NO:2, and the light chain variable region comprises at least 90% identity to a sequence set forth in SEQ ID NO:8.

In some such embodiments, the isolated antibody or antigen binding fragment thereof comprises an Fc polypeptide having at least 90% identity to a sequence of SEQ ID NO:63.

Also disclosed is a pharmaceutical composition comprising such isolated antibody or antigen binding fragment thereof and a pharmaceutically acceptable carrier; a diagnostic reagent comprising such isolated antibody or antigen binding fragment thereof; and, a kit comprising such isolated antibody or antigen binding fragment thereof or the diagnostic reagent.

Disclosed is an isolated nucleic acid comprising a nucleotide sequence that encodes a heavy chain variable region comprising a sequence set forth in SEQ ID NO: 14 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO: 14; and, an isolated nucleic acid comprising a nucleotide sequence that encodes a light chain variable region comprising a sequence set forth in SEQ ID NO:20 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:20. Also disclosed is an expression vector comprising the aforementioned nucleic acids, and an isolated host cell comprising the expression vector.

Disclosed herein is a method of detecting AXL, the method comprising: a) contacting a sample with the isolated antibody or antigen binding fragment thereof disclosed herein, under conditions to bind said antibody to an AXL on said sample, wherein the binding generates the production of a receptor/antibody complex; and, b) detecting the presence of the receptor/antibody complexes, wherein the detecting comprises the presence or absence of the AXL on said sample.

Also disclosed is a method of treating or preventing a disease or disorder associated with AXL in a subject, the method comprising: a) contacting a sample known or suspected to contain AXL with the isolated antibody or antigen binding fragment thereof disclosed herein; b) detecting the presence of complexes comprising AXL and the antibody; wherein the presence of the complexes indicates the presence of a disease or disorder; and, c) administering to the subject the isolated antibody or antigen binding fragment thereof that is disclosed herein.

Another disclosed method includes a method of diagnosing a disease or disorder, the method comprising: a) isolating a sample from a subject; b) incubating the sample with the isolated antibody or antigen binding fragment thereof disclosed herein, for a period of time sufficient to generate AXL:anti-AXL complexes; c) detecting the presence or absence of the AXL:anti-AXL complexes from the isolated tissue; and, d) associating presence or abundance of AXL with a location of interest of a tissue sample.

In one aspect, the disclosure provides an isolated antibody or antigen binding fragment thereof that specifically binds to AXL, wherein the isolated antibody comprises a) a heavy chain variable region comprising: (i) a heavy chain complementarity determining region 1 (CDRH1) comprising the sequence of X₁X₂X₃X₄X₅X₆ (SEQ ID NO:57), wherein X₁ is S, N or D, X₂ is A or Y, X₃ is Y or G, X₄ is S or M, X₅ is W, N or Y, and X₆ is H or no amino acid; (ii) a heavy chain complementarity determining region 2 (CDRH2) comprising the sequence of X₁IX₃X₄X₅X₆X₇X₈X₉X₁₀X₁X₁₂X₁₃X₁₄X₁₅X₁₆X₁₇ (SEQ ID NO:58), wherein X₁ is Y or W, X₃ is H, N or S, X₄ is Y, T or N, X₅ is S, Y or G, X₆ is G, I or T, X₇ is S or G, X₈ is T, E or S, X₉ is N, P or T, X₁₀ is Y or T, X₁₁ is N or Y, X₁₂ is Por A, X₁₃ is S or D, X₁₄ is L, D or T, X₁₅ is K, F or V, X₁₆ is S or K, and X₁₇ is G or no amino acid; (iii) a heavy chain complementarity determining region 3 (CDRH3) comprising the sequence of X₁X₂X₃FYAMDY (SEQ ID NO: 59), wherein X₁ is S, G or no amino acid, X₂ is L, T or D, and X₃ is W, T, R or H; and b) a light chain variable region comprising: (iv) a light chain complementarity determining region 1 (CDRL1) comprising the sequence of X₁ASX₄X₅X₆X₇X₈X₉X₁₀X₁X₁₂ (SEQ ID NO:60), wherein X₁ is R, H, S or K, X₄ is K, Q or S, X₅ is S, N or D, X₆ is I or V, X₇ is S or N, X₈ is K, V, S or T, X₉ is Y, V, S or T, X₁₀ is L, Y or V, X₁₁ is A, N or L, and X₁₂ is H or no amino acid; (v) a light chain complementarity determining region 2 (CDRL2) comprising the sequence of X₁X₂SX₄X₅X₆X₇ (SEQ ID NO:61), wherein X₁ is S, K, R or W, X₂ is G, A or T, X₄ is T or N, X₅ is L or R, X₆ is Q, H or A, X₇ is S or T; and (vi) a light chain complementarity determining region 3 (CDRL3) comprising the sequence of QQX₃X₄X₅X₆X₇X₈X₉ (SEQ ID NO:62), wherein X₃ is H or G, X₄ is N, Q, S or Y, X₅ is E, S or N, X₆ Y, I or T, X₇ is P, L or F, X₈ is W, L, T or F, X₉ is T or no amino acid.

In some embodiments, the CDRH1 comprises the sequence set forth in any of SEQ ID NOS: 24, 25, and 26 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 24, 25 and 26; the CDRH2 comprises the sequence set forth in any of SEQ ID NOS: 27, 28, 29 and 30 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 27, 28, 29 and 30; and the CDRH3 comprises the sequence set forth in any of SEQ ID NOS: 31, 32, 33 and 34 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 31, 32, 33 and 34.

In some embodiments, the CDRL1 comprises the sequence set forth in any one of SEQ ID NOS: 35, 36, 37 and 38 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 35, 36, 37 and 38; the CDRL2 comprises the sequence set forth in any one of SEQ ID NOS: 39, 40, 41, 42 and 43 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 39, 40, 41, 42 and 43; and the CDRL3 comprises the sequence set forth in any one of SEQ ID NOS: 44, 45, 46, 47 and 48 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 44, 45, 46, 47 and 48.

In some embodiments, the CDRH1 comprises the sequence set forth in any of SEQ ID NOS: 24, 25, and 26 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 24, 25 and 26; the CDRH2 comprises the sequence set forth in any of SEQ ID NOS: 27, 28, 29 and 30 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 27, 28, 29 and 30; the CDRH3 comprises the sequence set forth in any of SEQ ID NOS: 31, 32, 33 and 34 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 31, 32, 33 and 34, the CDRL1 comprises the sequence set forth in any one of SEQ ID NOS: 35, 36, 37 and 38 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 35, 36, 37 and 38; the CDRL2 comprises the sequence set forth in any one of SEQ ID NOS: 39, 40, 41, 42 and 43 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 39, 40, 41, 42 and 43; and the CDRL3 comprises the sequence set forth in any one of SEQ ID NOS: 44, 45, 46, 47 and 48 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 44, 45, 46, 47 and 48.

In some embodiments, the CDRH1 comprises the sequence set forth in SEQ ID NO: 24, the CDRH2 comprises the sequence set forth in SEQ ID NO:27, the CDRH3 comprises the sequence set forth in SEQ ID NO:31, the CDRL1 comprises the sequence set forth in SEQ ID NO: 36, the CDRL2 comprises the sequence set forth in SEQ ID NO:40, and the CDRL3 comprises the sequence set forth in SEQ ID NO:45.

In some embodiments, the heavy chain variable region comprises at least 90% identity to the sequence set forth in any one of SEQ ID NOS: 1-5. In some embodiments, the heavy chain variable region comprises at least 90% identity to the sequence set forth in SEQ ID NO: 1. In some embodiments, the light chain variable region comprises at least 90% identity to a sequence set forth in any of SEQ ID NOS: 6-11. In some embodiments, the light chain variable region comprises at least 90% identity to the sequence set forth in SEQ ID NO:7.

In some embodiments, the heavy chain variable region comprises at least 90% identity to the sequence set forth in any one of SEQ ID NOS: 1-5, and the light chain variable region comprises at least 90% identity to the sequence set forth in any of SEQ ID NOS: 6-11. In some embodiments, the heavy chain variable region comprises at least 90% identity to the sequence set forth in SEQ ID NO:1, and the light chain variable region comprises at least 90% identity to the sequence set forth in SEQ ID NO:7.

In some embodiments, any of the isolated antibodies or antigen binding fragments thereof further contains an Fc polypeptide having at least 90% identity to a sequence of SEQ ID NO: 63. In some embodiments, the antibody or antigen binding fragment thereof is monoclonal. In some embodiments, the antibody or antigen binding fragment thereof is humanized. In some embodiments, the isolated antibody or antigen binding fragment thereof further comprises one or more human framework regions. In some embodiments, the antibody is conjugated to a detectable marker or label. In some embodiments, the antibody is non-diffusively immobilized on a solid support.

Provided herein is an isolated nucleic acid encoding the isolated antibody or antigen binding fragment thereof of any one of embodiments described herein. In some embodiments, the isolated nucleic acid comprises a nucleotide sequence that encodes a heavy chain variable region comprising at least 90% sequence identity to the sequence set forth in any of SEQ ID NOS: 12-17. In some embodiments, the isolated nucleic acid comprises a nucleotide sequence that encodes a light chain variable region comprising at least 90% sequence identity to the sequence set forth in any of SEQ ID NOS: 18-23.

In another aspect, the disclosure provides an expression vector comprising the nucleic acid described herein.

In another aspect, the disclosure provides an isolated host cell comprising the expression vector described herein.

In another aspect, the disclosure provides a pharmaceutical composition comprising the isolated antibody or antigen binding fragment thereof described herein and a pharmaceutically acceptable carrier.

In another aspect, the disclosure provides a diagnostic reagent comprising the isolated antibody or antigen binding fragment thereof described herein.

In another aspect, the disclosure provides a kit comprising the isolated antibody or antigen binding fragment thereof described herein or the diagnostic reagent described herein.

In another aspect, the disclosure provides a method of detecting AXL comprising contacting a sample known or suspected to contain AXL with the isolated antibody or antigen binding fragment thereof described herein.

In another aspect, the disclosure provides a method of detecting AXL, wherein the method includes contacting a sample with the isolated antibody or antigen binding fragment thereof described herein, under conditions to bind said antibody to a AXL on said sample, wherein the binding generates the production of a receptor/antibody complex; and detecting the presence of the receptor/antibody complexes, wherein the detecting comprises the presence or absence of the AXL on said sample.

In another aspect, the disclosure provides a method of treating or preventing a disease or disorder associated with AXL in a subject, including contacting a sample known or suspected to contain AXL with the isolated antibody or antigen binding fragment thereof described herein; detecting the presence of complexes comprising AXL and the antibody; wherein the presence of the complexes indicates the presence of a disease or disorder; and administering to the subject the isolated antibody or antigen binding fragment thereof described herein.

In another aspect, the disclosure provides a method of diagnosing a disease or disorder, wherein the method includes isolating a sample from a subject; incubating the sample with the isolated antibody or antigen binding fragment thereof described herein, for a period of time sufficient to generate AXL:anti-AXL complexes; detecting the presence or absence of the AXL:anti-AXL complexes from the isolated tissue; and associating presence or abundance of AXL with a location of interest of a tissue sample.

In some of any embodiments of the methods described herein, the method is performed in vitro.

In some embodiments of the methods described herein, the detection comprises hybridization of a detectable moiety to the antibody or antigen binding fragment thereof. In some embodiments, the detectable moiety comprises an oligonucleotide. In some embodiments, the detectable moiety comprises a fluorescent label. In some embodiments, the detection comprises sequencing.

In some embodiments of the methods described herein, the sample comprises a cell. In some embodiments, the sample comprises a tissue sample.

Certain embodiments are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding or particular embodiments.

FIGS. 1A and 1B depict surface staining of antibodies AB1-AB6 on transfected BA/F3 cell lines. FIG. 1A depicts surface staining on BA/F3 cells transfected with human AXL, and FIG. 1B depicts surface staining on BA/F3 cells transfected with mGypa (mouse Glycophorin A) as a control. BA/F3 cells (IL-3 dependent mouse pro-B cell line) were used to eliminate the possibility of human proteins being expressed. The transfection efficiency was measured by GFP, which is used to visualize/identify cells that have been transfected and are expressing the vector. GFP (or FITC) on the x-axis identifies cells that are positive for GFP (i.e., cells that have been transfected). Six (6) purified antibodies, AB1-AB6, or isotype controls mIgG1 and mIgG2a, were used to stain the transfected cells for 15 min. Cells were then washed and stained with a secondary antibody (anti-mouse IgG-APC). Staining was measured by APC fluorescence, which was used to visualize and thus identify those cells to which each purified antibody AB1-AB6 and the corresponding second antibody, bound. As shown, APC on the y-axis identifies cells that are positive for APC fluorescence, thereby indicating cells that were bound by purified antibodies AB1-AB6. As shown in FIG. 1A, the cells stained with antibodies AB1-AB6 shift into the top right quadrant (“Q2”) (as compared to Q2 for isotype controls mIgG1 and mIgG2a), thereby demonstrating that each of antibody clones AB1-AB6 binds to cells transfected with human AXL (FIG. 1A), while FIG. 1B (top right quadrant, “Q2”) shows that none of the antibody clones AB1-AB6 binds to the mGypa control vector (as compared to Q2 for isotype controls mIgG1 and mIgG2a).

FIGS. 2A-2G depict cell surface staining of PE (phycoerythrin)-conjugated antibodies, PE-AB2-PE-AB6, across multiple human cell lines: A-431 (FIG. 2A), A-549 (FIG. 2B), HeLa (FIG. 2C), MDA-MB-231 (FIG. 2D) and NCI-H1299 (FIG. 2E) cells, TH-29 (FIG. 2F) and Jurkat (FIG. 2G), as compared to isotype controls. The cell lines used in FIGS. 2A-2E (i.e., A-431, A-549, HeLa, MDA-MB-231 and NCI-H1299, respectively) are known to express human AXL to varying levels. In contrast, the cell lines used for FIGS. 2F-2G (TH-29 and Jurkat, respectively) are known to be negative for human AXL and were thus used as negative controls. Data is represented as histograms with relative PE-fluorescence displayed on the x-axis and the cell count on the y-axis. For each of FIGS. 2A-2G, cell populations of interest were selected by setting a gate based on the corresponding isotype control and consistently displayed to facilitate comparison. In FIGS. 2A-2E, and using the gate as reference, all histograms corresponding to PE-labeled AB2-AB6 (and cell lines known to express (at varying levels) human AXL) indicate a shift of the single peak to the right on the x-axis when compared to the corresponding histogram of the isotype control for the same cell line. In contrast, no such shift is shown in FIGS. 2F-2G in relation to the two cell lines, respectively, known to be negative for human AXL. Accordingly, FIGS. 2A-2E demonstrate that PE-labeled AB2-AB6 bind to native human AXL (i.e., in contrast to FIG. 1, FIGS. 2A-2E are non-transfected cell lines) across all five demonstrated cell lines known to express human AXL at varying levels, while FIGS. 2F-2G indicate that PE-labeled AB2-AB6 do not bind to cells known to be negative for human AXL.

FIGS. 3B and 3D depict blocking or neutralization of PE-conjugated antibodies AB2-AB6 (FIG. 3B) and PU-conjugated AB1 (FIG. 3D) after pre-incubation with human AXL recombinant protein, as compared to no blocking or neutralization in the absence of pre-incubation with human AXL recombinant protein of PE-conjugated AB2-AB6 (FIG. 3A) and PU-conjugated AB1 (FIG. 3C). FIGS. 3A through 3D depict cell surface staining of either pre-incubated or non-pre-incubated PE-conjugated AB2-AB6 and PU-conjugated AB1 using BA/F3 cells that were transfected with human AXL recombinant protein. Data is represented as histograms with relative PE-fluorescence displayed on the x-axis and the cell count on the y-axis. In each case, cell populations of interest were selected by setting and displaying gates (for visual comparison purposes) based on the corresponding isotype control. The shift to the right (x-axis) of the peaks in FIGS. 3A and 3C of PE-conjugated AB2-AB6 and PU-conjugated AB1, as compared to the corresponding isotype control of such Figures, shows that without pre-incubation with human AXL recombinant protein, PE-conjugated AB2-AB6 and PU-conjugated AB1, respectively, stain the BA/F3 transfected cells. In contrast, the lack of shift to the right (x-axis) of peaks in FIGS. 3B and 3D shows that pre-incubating PE-conjugated AB1-AB6 and PU-conjugated AB1, respectively, with human AXL recombinant protein, before using PE-conjugated AB1-AB6 and PU-conjugated AB1 to stain the human AXL transfected BA/F3 cells, prevents PE-conjugated AB1-AB6 and PU-conjugated AB1 from staining (i.e., binding to) the BA/F3 transfected cells. FIGS. 3B and 3D thus demonstrate that recombinant human AXL protein blocks or neutralizes the ability of PE-conjugated AB2-AB6 and PU-conjugated AB1 from binding to the BA/F3 transfected cells.

FIGS. 4A and 4B depict different functional activities of antibodies AB2 and AB3 (disclosed herein) to block AXL receptor activation and subsequent downstream AKT (FIG. 4A) and ERK (FIG. 4B) phosphorylation induced by AXL's naturally occurring high affinity ligand, Gas6. H1299 cells were pre-incubated with buffer, AB2, AB3, or an isotype control. After pre-incubation at 37 degrees Celsius, Gas6 was added to selected tubes as depicted/labelled in FIGS. 4A and 4B (e.g., labels of “AB #+Gas6”, “Isotype+Gas6”, or “Gas6”, where #is either “2” or “3”). Control tubes were labelled “AB #”, “Isotype”, or “UT” (untreated), as shown in FIGS. 4A and 4B. As shown in FIG. 4A, left-hand side (AB2), “AB2” and “Isotype” controls caused minor AXL receptor activation-mediated p-AKT phosphorylation when compared to the “UT” control; however, very surprisingly, AB2 (see “AB2”, “AB2+Gas6”) fully blocked Gas6-induced p-AKT when compared to “Gas6”. “Isotype+Gas6” shows that the isotype has no blocking effect on Gas6 and is comparable to “Gas6”. With reference to FIG. 4B, left-hand side (AB2), a similar AB2 blocking effect is observed for Gas6-induced p-ERK phosphorylation, although in this instance, AB2 does not appear to fully block the p-ERK phosphorylation in the presence of the ligand, but greatly limits it (see increase in p-ERK phosphorylation of “AB2+Gas6” when compared to any of “UT”, “AB2”, and the “Isotype”). In contrast, no blocking effect was observed for p-AKT and/or p-ERK phosphorylation for AB3. As shown in FIG. 4A, right-hand side, “AB3” (and similarly to “AB2”, left-hand side) causes minor activation when compared to the “UT” and “Isotype” controls; however, unlike AB2, AB3 does not appear to block Gas6-induced p-AKT when compared to Gas6-only (see FIG. 4A, right-hand side, “AB3+Gas6” and “Gas6”). Data from “Isotype+Gas6” shows that the isotype has no blocking effect (compare with “Gas6”). A similar trend was observed for p-ERK phosphorylation (FIG. 4B, right-hand side), whereby AB3 does not appear to be able to block Gas6-induced p-ERK (compare “AB3+Gas6” and “Isotype+Gas6”). In summary, AB2 blocks Gas6-induced phosphorylation of p-AKT and greatly limits Gas6-induced phosphorylation of p-ERK. The surprising blocking activity of AB2 and inability of AB3 to block the AXL receptor activation allow for very distinct applications for each/both antibodies, including for example, therapeutic applications for AB2 and diagnostic and/or research tool applications for AB2 and AB3.

### DETAILED DESCRIPTION

Provided herein are antibodies and antigen-binding fragments thereof that bind AXL. For example, particular monoclonal antibodies to AXL that provide superior target specificity, signal-to-noise ratios, and the like as compared to other reported anti-AXL antibodies, as well as antigen-binding fragments of such antibodies that bind AXL, are described herein. Also provided herein are methods for producing anti-AXL antibodies and antigen-binding fragments thereof with desirable properties including affinity and/or specificity for AXL and/or its variants.

The following description includes information that may be useful in understanding the present technology. It is not an admission that any of the information provided herein, or any publication specifically or implicitly referenced herein, is prior art, or even particularly relevant, to the presently claimed technology.

### Antibody Generation and Characterization

Anti-AXL antibodies and antigen-binding fragments thereof provided herein may have a strong binding affinity and/or specificity for AXL. In some embodiments, anti-AXL antibodies and antigen-binding fragments thereof may be chimeric antibodies. In some embodiments, anti-AXL antibodies and antigen-binding fragments thereof may be humanized antibodies. In some embodiments, anti-AXL antibodies and antigen-binding fragments thereof may be variant antibodies. Antibodies, for example, may have beneficial properties from a therapeutic perspective. Assays for determining the activity of anti-AXL antibodies provided herein include, for example, cell-based ELISA (e.g., to measure cell specificity of the antibody), and cytotoxicity (e.g., to measure potential to mediate direct or indirect killing of AXL-expressing target cells). In certain instances, a humanized or variant antibody fails to elicit an immunogenic response upon administration of a therapeutically effective amount of the antibody to a human patient. In certain instances, if an immunogenic response is elicited, the response may be such that the antibody still provides a therapeutic benefit to the patient treated therewith.

In some embodiments, anti-AXL antibodies and antigen-binding fragments thereof (e.g., humanized anti-AXL antibodies) herein bind the same epitope. To screen for antibodies that bind to an epitope on AXL bound by an antibody of interest (e.g., those that block binding of the antibody to AXL), a cross-blocking assay such as that described in Antibodies, A Laboratory Manual, Cold Spring Harbor Laboratory, Ed Harlow and David Lane (1988), can be performed. In certain instances, epitope mapping, e.g., as described in Champe et al., J. Biol. Chem. 270:1388-1394 (1995), in Cunningham and Wells, Science 244:1081-1085 (1989), or in Davidson and Doranz, Immunology 143:13-20 (2014), can be performed to determine whether the antibody binds an epitope of interest.

Antibodies herein generally have a heavy chain variable domain comprising an amino acid sequence represented by the formula: FRH1-CDRH1-FRH2-CDRH2-FRH3-CDRH3-FRH4, where “FRH1-4” represents the four heavy chain framework regions and “CDRH1-3” represents the three hypervariable regions of an anti-AXL antibody variable heavy domain. FRH1-4 may be derived from a consensus sequence (for example the most common amino acids of a class, subclass or subgroup of heavy or light chains of human immunoglobulins) or may be derived from an individual human antibody framework region or from a combination of different framework region sequences. Many human antibody framework region or from a combination of different framework region sequences. Many human antibody framework regions sequences are compiled in Kabat et al. (1992) SEQUENCES OF PROTEINS OF IMMUNOLOGICAL INTEREST, National Institutes of Health Publication No. 91-3242, for example. In one embodiment, the variable heavy FR is provided by a consensus sequence of a human immunoglobulin subgroup as compiled by Kabat et al., supra.

The human variable heavy FR sequence may have substitutions therein, e.g., where the human FR residue is replaced by a corresponding nonhuman residue (by “corresponding nonhuman residue” is meant the nonhuman residue with the same Kabat positional numbering as the human residue of interest when the human and nonhuman sequences are aligned), but replacement with the nonhuman residue is not necessary. For example, a replacement FR residue other than the corresponding nonhuman residue may be selected by phage display.

Antibodies herein may have a light chain variable domain comprising an amino acid sequence represented by the formula: FRL1-CDRL1-FRL2-CDRL2-FRL3-CDRL3-FRL4, where “FRL1-4” represents the four framework regions and “CDRL1-3” represents the three hypervariable regions of an anti-AXL antibody variable light domain. FRL1-4 may be derived from a consensus sequence (for example the most common amino acids of a class, subclass or subgroup of heavy or light chains of human immunoglobulins) or may be derived from an individual human antibody framework region or from a combination of different framework region sequences. In one embodiment, the variable light FR is provided by a consensus sequence of a human immunoglobulin subgroup as compiled by Kabat et al., supra.

The human variable light FR sequence may have substitution therein, e.g., where the human FR residue is replaced by a corresponding mouse residue, but replacement with the nonhuman residue is not necessary. For example, a replacement residue other than the corresponding nonhuman residue may be selected by phage display. Methods for generating humanized anti-AXL antibodies of interest herein are elaborated in more detail below.

### Anti-AXL Antibodies and Antigen-Binding Fragments Thereof

Provided herein are antibodies and antigen-binding fragments thereof that bind AXL. Such antibodies and antigen-binding fragments thereof may include anti-AXL antibodies, anti-AXL antibody fragments (e.g., antigen-binding fragments), and anti-AXL antibody derivatives.

AXL is a member of the Tyro3-Axl-Mer (TAM) receptor tyrosine kinase subfamily. The encoded protein possesses an extracellular domain which is composed of two immunoglobulin-like motifs at the N-terminal, followed by two fibronectin type-III motifs. It transduces signals from the extracellular matrix into the cytoplasm by binding to the vitamin K-dependent protein growth arrest-specific 6 (Gas6). GAS6/AXL signaling functions as an important pathway driving cancer cell survival, proliferation, migration and invasion. The encoded protein acts as a host cell receptor for multiple viruses, including Marburg, Ebola and Lassa viruses and is a candidate receptor for the SARS-COV2 virus.

The term “AXL” as used herein refers to human AXL proteins, isoforms or variants thereof, including naturally occurring variants of human AXL, such as splice variants or allelic variants. The amino acid sequence of an exemplary human AXL is shown in SEQ ID NO: 56. In some embodiments, human AXL can refer to a variant, such as an allelic variant or splice variant, that exhibits at least or at least about 85%, 86%, 87%, 88%, 89%, 90%, 91%, 92%, 93%, 94%, 95%, 96%, 97%, 98%, or 99% sequence identity to a SEQ ID NOs: 56. In some embodiments, it is understood that the provided antibodies or antigen-binding fragments may exhibit cross-reactive binding to another mammalian AXL protein, such as murine AXL, or a primate AXL.

Human AXL amino acid sequence
(SEQ ID NO: 56)
MAWRCPRMGRVPLAWCLALCGWACMAPRGTQAEESPFVGNPGNITGARGLTGTLR
CQLQVQGEPPEVHWLRDGQILELADSTQTQVPLGEDEQDDWIVVSQLRITSLQLSDT
GQYQCLVFLGHQTFVSQPGYVGLEGLPYFLEEPEDRTVAANTPFNLSCQAQGPPEPV
DLLWLQDAVPLATAPGHGPQRSLHVPGLNKTSSFSCEAHNAKGVTTSRTATITVLPQ
QPRNLHLVSRQPTELEVAWTPGLSGIYPLTHCTLQAVLSDDGMGIQAGEPDPPEEPLT
SQASVPPHQLRLGSLHPHTPYHIRVACTSSQGPSSWTHWLPVETPEGVPLGPPENISA
TRNGSQAFVHWQEPRAPLQGTLLGYRLAYQGQDTPEVLMDIGLRQEVTLELQGDGS
VSNLTVCVAAYTAAGDGPWSLPVPLEAWRPGQAQPVHQLVKEPSTPAFSWPWWYV
LLGAVVAAACVLILALFLVHRRKKETRYGEVFEPTVERGELVVRYRVRKSYSRRTTE
ATLNSLGISEELKEKLRDVMVDRHKVALGKTLGEGEFGAVMEGQLNQDDSILKVAV
KTMKIAICTRSELEDELSEAVCMKEFDHPNVMRLIGVCFQGSERESFPAPVVILPFMK
HGDLHSFLLYSRLGDQPVYLPTQMLVKFMADIASGMEYLSTKRFIHRDLAARNCML
NENMSVCVADFGLSKKIYNGDYYRQGRIAKMPVKWIAIESLADRVYTSKSDVWSFG
VTMWEIATRGQTPYPGVENSEIYDYLRQGNRLKQPADCLDGLYALMSRCWELNPQ
DRPSFTELREDLENTLKALPPAQEPDEILYVNMDEGGGYPEPPGAAGGADPPTQPDP
KDSCSCLTAAEVHPAGRYVLCPSTTPSPAQPADRGSPAAPGQEDGA

In some embodiments, the antibody or antigen-binding fragment thereof is isolated (e.g., separated from a component of its natural environment (e.g., an animal, a biological sample)). In some embodiments, the antibody or antigen-binding fragment thereof is a humanized antibody or an antigen-binding fragment thereof. In some embodiments, the antibody or antigen-binding fragment thereof is a derivative of a humanized antibody that binds AXL. In some embodiments, the antibody or antigen-binding fragment thereof binds AXL under laboratory conditions (e.g., binds AXL in vitro, binds AXL in a flow cytometry assay, binds AXL in an ELISA). In some embodiments, the antibody or antigen-binding fragment thereof binds AXL under physiological conditions (e.g., binds AXL in a cell in a subject).

Generally, any of the anti-AXL antibodies or antigen-binding fragments thereof provided herein comprises at least one immunoglobulin heavy chain variable domain and at least one immunoglobulin light chain variable domain. In some embodiments, any of the anti-AXL antibodies or antigen-binding fragments thereof herein comprises two immunoglobulin heavy chain variable domains and two immunoglobulin light chain variable domains. Typically, each immunoglobulin heavy chain variable domain of the anti-AXL antibody or antigen-binding fragment thereof comprises first, second, and third heavy chain complementarity determining regions (CDRs; CDRH1, CDRH2, CDRH3), and each immunoglobulin light chain variable domain of the anti-AXL antibody or antigen-binding fragment thereof comprises first, second, and third light chain CDRs (CDRL1, CDRL2, CDRL3).

### CDRH1

In some embodiments, the CDRH1 of any of the anti-AXL antibodies or antigen-binding fragments thereof provided herein comprises an amino acid sequence chosen from SAYSWH (SEQ ID NO:24), NYGMN (SEQ ID NO:25), and DYYMY (SEQ ID NO:26) or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SAYSWH (SEQ ID NO: 24), NYGMN (SEQ ID NO:25), and DYYMY (SEQ ID NO:26). In some embodiments, the CDRH1 of any of the anti-AXL antibodies or antigen-binding fragments thereof provided herein comprises an amino acid sequence chosen from SAYSWH (SEQ ID NO:24), NYGMN (SEQ ID NO:25), and DYYMY (SEQ ID NO:26).

In some embodiments, the CDRH1 of any of the anti-AXL antibodies or antigen-binding fragments thereof provided herein comprises the amino acid sequence of X₁X₂X₃X₄X₅X₆ (SEQ ID NO:57), in which X₁ is S, N or D, X₂ is A or Y, X₃ is Y or G, X₄ is S or M, X₅ is W, N or Y, and X₆ is H or no amino acid.

### CDRH2

In some embodiments, the CDRH2 of any of the anti-AXL antibodies or antigen-binding fragments thereof provided herein comprises an amino acid sequence chosen from YIHYSGSTNYNPSLKS (SEQ ID NO:27), WINTYIGEPTYADDFKG (SEQ ID NO:28), WINTYTGEPTYADDFKG (SEQ ID NO:29) and YISNGGGSTYYPDTVKG (SEQ ID NO: 30) or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of YIHYSGSTNYNPSLKS (SEQ ID NO:27), WINTYIGEPTYADDFKG (SEQ ID NO:28), WINTYTGEPTYADDFKG (SEQ ID NO:29) and YISNGGGSTYYPDTVKG (SEQ ID NO: 30). In some embodiments, the CDRH2 of any of the anti-AXL antibodies or antigen-binding fragments thereof provided herein comprises an amino acid sequence chosen from YIHYSGSTNYNPSLKS (SEQ ID NO:27), WINTYIGEPTYADDFKG (SEQ ID NO:28), WINTYTGEPTYADDFKG (SEQ ID NO:29) and YISNGGGSTYYPDTVKG (SEQ ID NO: 30)

In some embodiments, the CDRH2 of any of the anti-AXL antibodies or antigen-binding fragments thereof provided herein comprises the amino acid sequence of X₁IX₃X₄X₅X₆X₇X₈X₉X₁₀X₁X₁₂X₁₃X₁₄X₁₅X₁₆X₁₇ (SEQ ID NO:58), X₁ is Y or W, X₃ is H, N or S, X₄ is Y, T or N, X₅ is S, Y or G, X₆ is G, I or T, X₇ is S or G, X₈ is T, E or S, X₉ is N, P or T, X₁₀ is Y or T, X₁₁ is N or Y, X₁₂ is Por A, X₁₃ is S or D, X₁₄ is L, D or T, X₁₅ is K, F or V, X₁₆ is S or K, and X₁₇ is G or no amino acid.

### CDRH3

In some embodiments, the CDRH3 of any of the anti-AXL antibodies or antigen-binding fragments t thereof provided herein comprises an amino acid sequence chosen from SLWFYAMDY (SEQ ID NO:31), GTTFYAMDY (SEQ ID NO:32), GDRFYAMDY (SEQ ID NO: 33) and LHFYAMDY (SEQ ID NO:34) or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SLWFYAMDY (SEQ ID NO:31), GTTFYAMDY (SEQ ID NO: 32), GDRFYAMDY (SEQ ID NO:33) and LHFYAMDY (SEQ ID NO:34) . . . . In some embodiments, the CDRH3 of any of the anti-AXL antibodies or antigen-binding fragments t thereof provided herein comprises an amino acid sequence chosen from SLWFYAMDY (SEQ ID NO: 31), GTTFYAMDY (SEQ ID NO:32), GDRFYAMDY (SEQ ID NO:33) and LHFYAMDY (SEQ ID NO:34).

In some embodiments, the CDRH3 of any of the anti-AXL antibodies or antigen-binding fragments thereof provided herein comprises the amino acid sequence of X₁X₂X₃FYAMDY (SEQ ID NO:59), in which X₁ is S, G or no amino acid, X₂ is L, T or D, and X₃ is W, T, R or H.

### CDRL1

In some embodiments, the CDRL1 of any of the anti-AXL antibodies or antigen-binding fragments thereof provided herein comprises an amino acid sequence chosen from RASKSISKYLA (SEQ ID NO:35), HASQNINVWLN (SEQ ID NO:36), SASSSISSNYLH (SEQ ID NO: 37) and KASQDVSTAVA (SEQ ID NO:38) or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of RASKSISKYLA (SEQ ID NO:35), HASQNINVWLN (SEQ ID NO:36), SASSSISSNYLH (SEQ ID NO: 37) and KASQDVSTAVA (SEQ ID NO:38. In some embodiments, the CDRL1 of any of the anti-AXL antibodies or antigen-binding fragments thereof provided herein comprises an amino acid sequence chosen from RASKSISKYLA (SEQ ID NO:35), HASQNINVWLN (SEQ ID NO: 36), SASSSISSNYLH (SEQ ID NO: 37) and KASQDVSTAVA (SEQ ID NO:38)

In some embodiments, the CDRL1 of any of the anti-AXL antibodies or antigen-binding fragments thereof provided herein comprises the amino acid sequence of X₁ASX₄X₅X₆X₇X₈X₉X₁₀X₁X₁₂ (SEQ ID NO:60), in which X₁ is R, H, S or K, X₄ is K, Q or S, X₅ is S, N or D, X₆ is I or V, X₇ is S or N, X₈ is K, V, S or T, X₉ is Y, V, S or T, X₁₀ is L, Y or V, X₁₁ is A, N or L, and X₁₂ is H or no amino acid.

### CDRL2

In some embodiments, the CDRL2 of any of the anti-AXL antibodies or antigen-binding fragments thereof provided herein comprises an amino acid sequence chosen from SGSTLQS (SEQ ID NO:39), KASNLHT (SEQ ID NO:40), RTSNLAS (SEQ ID NO:41), SGSTLHS (SEQ ID NO:42), and WASTRHT (SEQ ID NO:43) or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SGSTLQS (SEQ ID NO:39), KASNLHT (SEQ ID NO:40), RTSNLAS (SEQ ID NO:41), SGSTLHS (SEQ ID NO:42), and WASTRHT (SEQ ID NO:43). In some embodiments, the CDRL2 of any of the anti-AXL antibodies or antigen-binding fragments thereof provided herein comprises an amino acid sequence chosen from SGSTLQS (SEQ ID NO:39), KASNLHT (SEQ ID NO:40), RTSNLAS (SEQ ID NO:41), SGSTLHS (SEQ ID NO:42), and WASTRHT (SEQ ID NO:43).

In some embodiments, the CDRL2 of any of the anti-AXL antibodies or antigen-binding fragments thereof provided herein comprises the amino acid sequence of X₁X₂SX₄X₅X₆X₇ (SEQ ID NO:61), in which X₁ is S, K, R or W, X₂ is G, A or T, X₄ is T or N, X₅ is L or R, X₆ is Q, H or A, X₇ is S or T.

### CDRL3

In some embodiments, the CDRL3 of any of the anti-AXL antibodies or antigen-binding fragments thereof provided herein comprises an amino acid sequence chosen from QQHNEYPWT (SEQ ID NO:44), QQGQSYPLT (SEQ ID NO:45), QQGSSILT (SEQ ID NO: 46), QQGSSIFT (SEQ ID NO:47) and QQHYNTPFT (SEQ ID NO:48) or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of QQHNEYPWT (SEQ ID NO: 44), QQGQSYPLT (SEQ ID NO:45), QQGSSILT (SEQ ID NO:46), QQGSSIFT (SEQ ID NO: 47) and QQHYNTPFT (SEQ ID NO:48. In some embodiments, the CDRL3 of any of the anti-AXL antibodies or antigen-binding fragments thereof provided herein comprises an amino acid sequence chosen from QQHNEYPWT (SEQ ID NO:44), QQGQSYPLT (SEQ ID NO:45), QQGSSILT (SEQ ID NO:46), QQGSSIFT (SEQ ID NO:47) and QQHYNTPFT (SEQ ID NO: 48).

In some embodiments, the CDRL3 of any of the anti-AXL antibodies or antigen-binding fragments thereof provided herein comprises the amino acid sequence of QQX₃X₄X₅X₆X₇X₈X₉ (SEQ ID NO:62), in which X₃ is H or G, X₄ is N, Q, S or Y, X₅ is E, S or N, X₆ Y, I or T, X₇ is P, L or F, X₈ is W, L, T or F, X₉ is T or no amino acid.

### CDR Sets

In some embodiments, any of the anti-AXL antibodies or antigen-binding fragments thereof described herein comprises an immunoglobulin heavy chain variable domain comprising a set of CDRs (i.e., CDRH1, CDRH2, CDRH3); and an immunoglobulin light chain variable domain comprising a set of CDRs (i.e., CDRL1, CDRL2, CDRL3). In some embodiments, an anti-AXL antibody or antigen-binding fragment thereof herein comprises two immunoglobulin heavy chain variable domains each comprising a set of CDRs (i.e., CDRH1, CDRH2, CDRH3); and two immunoglobulin light chain variable domains each comprising a set of CDRs (i.e., CDRL1, CDRL2, CDRL3). Sets of CDRs may comprise any combination of CDR amino acid sequences (i.e., CDRH1, CDRH2, CDRH3; and CDRL1, CDRL2, CDRL3) provided herein. In some embodiments, an immunoglobulin heavy chain variable domain comprises a set of CDRH1, CDRH2, and CDRH3 amino acid sequences, and an immunoglobulin light chain variable domain comprises a set of CDRL1, CDRL2, and CDRL3 amino acid sequences chosen from sets 1-6 provided in the following table.

TABLE 1
CDR Sets

| | CDRH1 | CDRH2 | CDRH3 | CDRL1 | CDRL2 | CDRL3 |
|---|---|---|---|---|---|---|
| | (SEQ ID | (SEQ ID | (SEQ ID | (SEQ ID | (SEQ ID | (SEQ ID |
| Ab | NO:) | NO:) | NO:) | NO:) | NO:) | NO:) |
| 1 | SAYSWH | YIHYSGSTNY | SLWFYAMDY | RASKSISKYL | SGSTLQS | QQHNEYPWT |
| | (SEQ ID | NPSLKS (SEQ | (SEQ ID | A (SEQ ID | (SEQ ID | (SEQ ID NO: |
| | NO: 24) | ID NO: 27) | NO: 31) | NO: 35) | NO: 39) | 44) |
| 2 | SAYSWH | YIHYSGSTNY | SLWFYAMDY | HASQNINVW | KASNLHT | QQGQSYPLT |
| | (SEQ ID | NPSLKS (SEQ | (SEQ ID | LN (SEQ ID | (SEQ ID | (SEQ ID NO: |
| | NO: 24) | ID NO: 27) | NO: 31) | NO: 36) | NO:40) | 45) |
| 3 | NYGMN (SEQ | WINTYIGEPT | GTTFYAMDY | SASSSISSNYL | RTSNLAS | QQGSSILT |
| | ID NO: 25) | YADDFKG | (SEQ ID | H (SEQ ID | (SEQ ID | (SEQ ID NO: |
| | | (SEQ ID | NO: 32) | NO: 37) | NO: 41) | 46) |
| | | NO: 28) | | | | |
| 4 | SAYSWH | YIHYSGSTNY | SLWFYAMDY | RASKSISKYL | SGSTLHS | QQHNEYPWT |
| | (SEQ ID | NPSLKS (SEQ | (SEQ ID | A (SEQ ID | (SEQ ID | (SEQ ID NO: |
| | NO: 24) | ID NO: 27) | NO: 31) | NO: 35) | NO: 42) | 44) |
| 5 | NYGMN | WINTYTGEPT | GDRFYAMDY | SASSSISSNYL | RTSNLAS | QQGSSIFT |
| | (SEQ ID | YADDFKG | (SEQ ID | H (SEQ ID | (SEQ ID NO: | (SEQ ID |
| | NO: 25) | (SEQ ID | NO: 33) | NO: 37) | 41) | NO: 47) |
| | | NO: 29) | | | | |
| 6 | DYYMY | YISNGGGSTY | LHFYAMDY | KASQDVSTA | WASTRHT | QQHYNTPFT |
| | (SEQ ID | YPDTVKG | (SEQ ID NO: | VA (SEQ ID | (SEQ ID | (SEQ ID |
| | NO: 26) | (SEQ ID | 34) | NO: 38) | NO: 43) | NO: 48) |
| | | NO: 30) | | | | |

In some embodiments, any of the antibodies provided herein includes a CDRH1 containing the sequence set forth in SEQ ID NO:24 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:24, a CDRH2 containing the sequence set forth in SEQ ID NO:27, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:27, a CDRH3 containing the sequence set forth in SEQ ID NO:31, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:31, a CDRL1 containing the sequence set forth in SEQ ID NO:35, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:35, a CDRL2 containing the sequence set forth in SEQ ID NO: 39, and a CDRL3 containing the sequence set forth in SEQ ID NO:44, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:44.

In some embodiments, any of the antibodies provided herein includes a CDRH1 containing the sequence set forth in SEQ ID NO:24, a CDRH2 containing the sequence set forth in SEQ ID NO:27, a CDRH3 containing the sequence set forth in SEQ ID NO:31, a CDRL1 containing the sequence set forth in SEQ ID NO:35, a CDRL2 containing the sequence set forth in SEQ ID NO:39, and a CDRL3 containing the sequence set forth in SEQ ID NO:44.

In some embodiments, any of the antibodies provided herein includes a CDRH1 containing the sequence set forth in SEQ ID NO:24, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:24, a CDRH2 containing the sequence set forth in SEQ ID NO:27, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:27, a CDRH3 containing the sequence set forth in SEQ ID NO:31, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:31, a CDRL1 containing the sequence set forth in SEQ ID NO:36, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:36, a CDRL2 containing the sequence set forth in SEQ ID NO: 40, and a CDRL3 containing the sequence set forth in SEQ ID NO:45 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:45.

In some embodiments, any of the antibodies provided herein includes a CDRH1 containing the sequence set forth in SEQ ID NO:24, a CDRH2 containing the sequence set forth in SEQ ID NO:27, a CDRH3 containing the sequence set forth in SEQ ID NO:31, a CDRL1 containing the sequence set forth in SEQ ID NO:36, a CDRL2 containing the sequence set forth in SEQ ID NO:40, and a CDRL3 containing the sequence set forth in SEQ ID NO:45.

In some embodiments, any of the antibodies provided herein includes a CDRH1 containing the sequence set forth in SEQ ID NO:25, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:25, a CDRH2 containing the sequence set forth in SEQ ID NO:28, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:28, a CDRH3 containing the sequence set forth in SEQ ID NO:32, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:32, a CDRL1 containing the sequence set forth in SEQ ID NO:37, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:37, a CDRL2 containing the sequence set forth in SEQ ID NO: 41, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 41, and a CDRL3 containing the sequence set forth in SEQ ID NO:46 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:46.

In some embodiments, any of the antibodies provided herein includes a CDRH1 containing the sequence set forth in SEQ ID NO:25, a CDRH2 containing the sequence set forth in SEQ ID NO:28, a CDRH3 containing the sequence set forth in SEQ ID NO:32, a CDRL1 containing the sequence set forth in SEQ ID NO:37, a CDRL2 containing the sequence set forth in SEQ ID NO:41, and a CDRL3 containing the sequence set forth in SEQ ID NO:46.

In some embodiments, any of the antibodies provided herein includes a CDRH1 containing the sequence set forth in SEQ ID NO:24, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 24, a CDRH2 containing the sequence set forth in SEQ ID NO:27, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:27, a CDRH3 containing the sequence set forth in SEQ ID NO:31, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 31, a CDRL1 containing the sequence set forth in SEQ ID NO:35, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:35, a CDRL2 containing the sequence set forth in SEQ ID NO: 42, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 42, and a CDRL3 containing the sequence set forth in SEQ ID NO:44, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:44.

In some embodiments, any of the antibodies provided herein includes a CDRH1 containing the sequence set forth in SEQ ID NO:24, a CDRH2 containing the sequence set forth in SEQ ID NO:27, a CDRH3 containing the sequence set forth in SEQ ID NO:31, a CDRL1 containing the sequence set forth in SEQ ID NO:35, a CDRL2 containing the sequence set forth in SEQ ID NO:42, and a CDRL3 containing the sequence set forth in SEQ ID NO:44.

In some embodiments, any of the antibodies provided herein includes a CDRH1 containing the sequence set forth in SEQ ID NO:25, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:25, a CDRH2 containing the sequence set forth in SEQ ID NO:29, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 29 a CDRH3 containing the sequence set forth in SEQ ID NO:33, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 33, a CDRL1 containing the sequence set forth in SEQ ID NO:37, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:37, a CDRL2 containing the sequence set forth in SEQ ID NO: 41, and a CDRL3 containing the sequence set forth in SEQ ID NO:47, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:47.

In some embodiments, any of the antibodies provided herein includes a CDRH1 containing the sequence set forth in SEQ ID NO:25, a CDRH2 containing the sequence set forth in SEQ ID NO:29, a CDRH3 containing the sequence set forth in SEQ ID NO:33, a CDRL1 containing the sequence set forth in SEQ ID NO:37, a CDRL2 containing the sequence set forth in SEQ ID NO:41, and a CDRL3 containing the sequence set forth in SEQ ID NO:47.

In some embodiments, any of the antibodies provided herein includes a CDRH1 containing the sequence set forth in SEQ ID NO:26, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 26, a CDRH2 containing the sequence set forth in SEQ ID NO:30, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:30, a CDRH3 containing the sequence set forth in SEQ ID NO:34, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:34, a CDRL1 containing the sequence set forth in SEQ ID NO:38, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:38, a CDRL2 containing the sequence set forth in SEQ ID NO: 43, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 43, and a CDRL3 containing the sequence set forth in SEQ ID NO:48, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:48.

In some embodiments, any of the antibodies provided herein includes a CDRH1 containing the sequence set forth in SEQ ID NO:26, a CDRH2 containing the sequence set forth in SEQ ID NO:30, a CDRH3 containing the sequence set forth in SEQ ID NO:34, a CDRL1 containing the sequence set forth in SEQ ID NO:38, a CDRL2 containing the sequence set forth in SEQ ID NO:43, and a CDRL3 containing the sequence set forth in SEQ ID NO:48.

In some embodiments, all CDRs are from the same set. For example, for an anti-AXL antibody or antigen-binding fragment thereof comprising two immunoglobulin heavy chain variable domains and two immunoglobulin light chain variable domains, each immunoglobulin heavy chain variable domain may comprise a set of CDRH1, CDRH2, and CDRH3 amino acid sequences from set 1, and each immunoglobulin light chain variable domain may comprise a set of CDRL1, CDRL2, and CDRL3 amino acid sequences from set 1.

In some embodiments, CDRs are from different sets. For example, for an anti-AXL antibody or antigen-binding fragment thereof comprising two immunoglobulin heavy chain variable domains and two immunoglobulin light chain variable domains, each immunoglobulin heavy chain variable domain may comprise a set of CDRH1, CDRH2, and CDRH3 amino acid sequences from set 1, and each immunoglobulin light chain variable domain may comprise a set of CDRL1, CDRL2, and CDRL3 amino acid sequences from set 2. In another example, for an anti-AXL antibody or antigen-binding fragment thereof comprising two immunoglobulin heavy chain variable domains and two immunoglobulin light chain variable domains, one immunoglobulin heavy chain variable domain may comprise a set of CDRH1, CDRH2, and CDRH3 amino acid sequences from set 2; and one immunoglobulin light chain variable domain may comprise a set of CDRL1, CDRL2, and CDRL3 amino acid sequences from set 1 and other immunoglobulin light chain variable domain may comprise a set of CDRL1, CDRL2, and CDRL3 amino acid sequences from set 2.

### VH

In some embodiments, any of the anti-AXL antibodies or antigen-binding fragments thereof provided herein may comprise a heavy chain variable domain (VH). In some embodiments, a heavy chain variable domain (VH) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence DVQLQESGPDLVKPSQSLSLTCTVTGYSITSAYSWHWIRQFPGNKLEWMGYIHYSGS TNYNPSLKSRISITRDTSKNQFFLQLNSVTTEDTATYYCARSLWFYAMDYWGQGTSV TVSS (SEQ ID NO:1), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:1. In some embodiments, a VH comprises a polypeptide that is at least 90% identical to the amino acid sequence of SEQ ID NO:1. In some embodiments, a VH comprises a polypeptide that is at least 95% identical to the amino acid sequence of SEQ ID NO:1. In some embodiments, a VH comprises a polypeptide that is 100% identical to the amino acid sequence of SEQ ID NO: 1.

In some embodiments, a heavy chain variable domain (VH) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence QIQLVQSGPELKKPGETVKISCKASGYTFTNYGMNWVKQAPGKGLKWMGWINTYI GEPTYADDFKGRFAFSLETSASTAYLQINNLKNEDMGTYFCARGTTFYAMDYWGQ GTSVTVSSS (SEQ ID NO:2), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO: 2. In some embodiments, a VH comprises a polypeptide that is at least 90% identical to the amino acid sequence of SEQ ID NO:2. In some embodiments, a VH comprises a polypeptide that is at least 95% identical to the amino acid sequence of SEQ ID NO:2. In some embodiments, a VH comprises a polypeptide that is 100% identical to the amino acid sequence of SEQ ID NO:2.

In some embodiments, a heavy chain variable domain (VH) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence DVQLQESGPDL VKPSQSLSLTCTVTGYSITSAYSWHWIRQFPGNKLEWMGYIHYSGS TNYNPSLKSRISITRDTSKNQFFLQLSSVTTEDTATYYCARSLWFYAMDYWGQGTSV TVSS (SEQ ID NO:3), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:3. In some embodiments, a VH comprises a polypeptide that is at least 90% identical to the amino acid sequence of SEQ ID NO:3. In some embodiments, a VH comprises a polypeptide that is at least 95% identical to the amino acid sequence of SEQ ID NO:3. In some embodiments, a VH comprises a polypeptide that is 100% identical to the amino acid sequence of SEQ ID NO: 3.

In some embodiments, a heavy chain variable domain (VH) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence QIQLVQSGPELKKPGETVKISCKASGYTFTNYGMNWVKQAPGKGLKWMGWINTYT GEPTYADDFKGRFAFSLETSASTAYLQINNLKNEDMATYFCARGDRFYAMDYWGQ GTSVTVSS (SEQ ID NO:4), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO: 4. In some embodiments, a VH comprises a polypeptide that is at least 90% identical to the amino acid sequence of SEQ ID NO:4. In some embodiments, a VH comprises a polypeptide that is at least 95% identical to the amino acid sequence of SEQ ID NO:4. In some embodiments, a VH comprises a polypeptide that is 100% identical to the amino acid sequence of SEQ ID NO:4.

In some embodiments, a heavy chain variable domain (VH) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence EVNLVESGGGLVQPGGSLKLSCATSGFTFSDYYMYWVRQTPEKRLEWVAYISNGGG STYYPDTVKGRFTISRDNAKNTLYLQMSRLKSEDTAMYYCARLHFYAMDYWGQGT SVTVSSS (SEQ ID NO:5), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:5. In some embodiments, a VH comprises a polypeptide that is at least 90% identical to the amino acid sequence of SEQ ID NO:5. In some embodiments, a VH comprises a polypeptide that is at least 95% identical to the amino acid sequence of SEQ ID NO:5. In some embodiments, a VH comprises a polypeptide that is 100% identical to the amino acid sequence of SEQ ID NO: 5.

In some embodiments, a VH of an anti-AXL antibody or antigen-binding fragment thereof provided herein comprises a polypeptide chosen from SEQ ID NO:1, SEQ ID NO:2, SEQ ID NO:3, SEQ ID NO:4, and SEQ ID NO:5.

In some embodiments, the VH of any of the anti-AXL antibodies or antigen binding fragments thereof provided herein further comprises a signal sequence. In some embodiments, VH signal sequence of any of the anti-AXL antibodies or antigen binding fragments thereof provided herein comprises the sequence of amino acids set forth in SEQ ID NO:49, SEQ ID NO: 50 or SEQ ID NO:51. In some embodiments, VH signal sequence of any of the anti-AXL antibodies or antigen binding fragments thereof provided herein comprises the sequence of amino acids set forth in SEQ ID NO:49. In some embodiments, VH signal sequence of any of the anti-AXL antibodies or antigen binding fragments thereof provided herein comprises the sequence of amino acids set forth in SEQ ID NO:50. In some embodiments, VH signal sequence of any of the anti-AXL antibodies or antigen binding fragments thereof provided herein comprises the sequence of amino acids set forth in SEQ ID NO:51.

### VL

In some embodiments, any of the anti-AXL antibodies or antigen-binding fragments thereof provided herein may comprise a light chain variable domain (VL). In some embodiments, a light chain variable domain (VL) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence DVQITQSPSYLAASPGETITINCRASKSISKYLAWYQEKPGKTNKLLIYSGSTLQSGIPS RFSGSGSGTDFTLTISSLEPEDFAMYYCQQHNEYPWTFGGGTKLEIK (SEQ ID NO:6), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:6. In some embodiments, a VL comprises a polypeptide that is at least 90% identical to the amino acid sequence of SEQ ID NO: 6. In some embodiments, a VL comprises a polypeptide that is at least 95% identical to the amino acid sequence of SEQ ID NO:6. In some embodiments, a VL comprises a polypeptide that is 100% identical to the amino acid sequence of SEQ ID NO:6.

In some embodiments, a light chain variable domain (VL) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or identical the amino 100%) to acid sequence DIQMNQSPSSLSASLGDTITITCHASQNINVWLNWYQQKPGNIPKLLIYKASNLHTGV PSRFSGSGSGTGFTLTISSLQPEDIATYYCQQGQSYPLTFGGGTKLEIK (SEQ ID NO:7), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:7. In some embodiments, a VL comprises a polypeptide that is at least 90% identical to the amino acid sequence of SEQ ID NO: 7. In some embodiments, a VL comprises a polypeptide that is at least 95% identical to the amino acid sequence of SEQ ID NO:7. In some embodiments, a VL comprises a polypeptide that is 100% identical to the amino acid sequence of SEQ ID NO:7.

In some embodiments, a light chain variable domain (VL) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence EIVLTQSPTTMAASPGEKITITCSASSSISSNYLHWYQQKPGFSPKLLIYRTSNLASGVP ARFSGSGSGTSYSLTIGTMEAEDVATYYCQQGSSILTFGAGTKLELK (SEQ ID NO:8), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:8. In some embodiments, a VL comprises a polypeptide that is at least 90% identical to the amino acid sequence of SEQ ID NO: 8. In some embodiments, a VL comprises a polypeptide that is at least 95% identical to the amino acid sequence of SEQ ID NO:8. In some embodiments, a VL comprises a polypeptide that is 100% identical to the amino acid sequence of SEQ ID NO:8.

In some embodiments, a light chain variable domain (VL) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence DVQITQSPSYLAASPGETITIKCRASKSISKYLAWYQEKPGKTNKLLIYSGSTLHSGIPS RFSGSGSGTDFTLTISSLEPEDFAMYYCQQHNEYPWTFGGGTKLEIK (SEQ ID NO:9), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:9. In some embodiments, a VL comprises a polypeptide that is at least 90% identical to the amino acid sequence of SEQ ID NO: 9. In some embodiments, a VL comprises a polypeptide that is at least 95% identical to the amino acid sequence of SEQ ID NO:9. In some embodiments, a VL comprises a polypeptide that is 100% identical to the amino acid sequence of SEQ ID NO:9.

In some embodiments, a light chain variable domain (VL) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or 100%) identical to the amino acid sequence EIVLTQSPTTMAASPGEKITITCSASSSISSNYLHWYQQKPGFSPKLLIYRTSNLASGVP TRFSGSGSGTSYSLTIGTMEAEDVATYYCQQGSSIFTFGSGTKLEIK (SEQ ID NO:10), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:10. In some embodiments, a VL comprises a polypeptide that is at least 90% identical to the amino acid sequence of SEQ ID NO: 10. In some embodiments, a VL comprises a polypeptide that is at least 95% identical to the amino acid sequence of SEQ ID NO:10. In some embodiments, a VL comprises a polypeptide that is 100% identical to the amino acid sequence of SEQ ID NO:10.

In some embodiments, a light chain variable domain (VL) of any of the anti-AXL antibodies or antigen-binding fragments thereof provided herein comprises a polypeptide that is at least 80% (e.g., 80%, 82%, 84%, 86%, 88%, 90%, 91%, 92%, 93%, 94%, 95%, 96%, 97%, 98%, 99%, or identical the 100%) to amino acid sequence DIVMTQSHKFMSTSVGDRVSITCKASQDVSTAVAWYQQKPGQSPKLLIYWASTRHT GVPDRFTGSGSGTDYTLTISSVQAEDLALYYCQQHYNTPFTFGSGTKLEIK (SEQ ID NO: 11), e.g., 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, identical to the amino acid sequence of SEQ ID NO:11. In some embodiments, a VL comprises a polypeptide that is at least 90% identical to the amino acid sequence of SEQ ID NO:11. In some embodiments, a VL comprises a polypeptide that is at least 95% identical to the amino acid sequence of SEQ ID NO:11. In some embodiments, a VL comprises a polypeptide that is 100% identical to the amino acid sequence of SEQ ID NO: 11.

In some embodiments, a VL of an anti-AXL antibody or antigen-binding fragment thereof provided herein comprises a polypeptide chosen from SEQ ID NO:6, SEQ ID NO:7, SEQ ID NO:8, SEQ ID NO:9, SEQ ID NO: 10, and SEQ ID NO:11.

In some embodiments, the VL of any of the anti-AXL antibodies or antigen binding fragments thereof provided herein further comprises a signal sequence. In some embodiments, VL signal sequence of any of the anti-AXL antibodies or antigen binding fragments thereof provided herein comprises the sequence of amino acids set forth in SEQ ID NO:52, SEQ ID NO: 53, SEQ ID NO:54 or SEQ ID NO:55. In some embodiments, VL signal sequence of any of the anti-AXL antibodies or antigen binding fragments thereof provided herein comprises the sequence of amino acids set forth in SEQ ID NO:52. In some embodiments, VL signal sequence of any of the anti-AXL antibodies or antigen binding fragments thereof provided herein comprises the sequence of amino acids set forth in SEQ ID NO:53. In some embodiments, VL signal sequence of any of the anti-AXL antibodies or antigen binding fragments thereof provided herein comprises the sequence of amino acids set forth in SEQ ID NO:54. In some embodiments, VL signal sequence of any of the anti-AXL antibodies or antigen binding fragments thereof provided herein comprises the sequence of amino acids set forth in SEQ ID NO: 55.

### VH and VL Combinations

In some of any embodiments, any of the isolated antibodies or antigen binding fragment thereof provided herein contains a heavy chain variable region with at least 90% identity to the sequence set forth in any one of SEQ ID NOs: 1-5, and the light chain variable region contains at least 90% identity to the sequence set forth in any of SEQ ID NOs: 6-11.

In some of any embodiments, the heavy chain variable region comprises at least 90% identity to the sequence set forth in SEQ ID NO:1, and the light chain variable region comprises at least 90% identity to the sequence set forth in SEQ ID NO:6. In some of any embodiments, the heavy chain variable region comprises at least 90% identity to the sequence set forth in SEQ ID NO:1, and the light chain variable region comprises at least 90% identity to the sequence set forth in SEQ ID NO:7. In some of any embodiments, the heavy chain variable region comprises at least 90% identity to the sequence set forth in SEQ ID NO:2, and the light chain variable region comprises at least 90% identity to the sequence set forth in SEQ ID NO:8. In some of any embodiments, the heavy chain variable region comprises at least 90% identity to the sequence set forth in SEQ ID NO:3, and the light chain variable region comprises at least 90% identity to the sequence set forth in SEQ ID NO:9. In some of any embodiments, the heavy chain variable region comprises at least 90% identity to the sequence set forth in SEQ ID NO:4, and the light chain variable region comprises at least 90% identity to the sequence set forth in SEQ ID NO:10. In some of any embodiments, the heavy chain variable region comprises at least 90% identity to the sequence set forth in SEQ ID NO: 5, and the light chain variable region comprises at least 90% identity to the sequence set forth in SEQ ID NO:11.

In some of any embodiments, any of the isolated antibodies or antigen binding fragment thereof provided herein contains a heavy chain variable region that comprises the sequence set forth in any one of SEQ ID NOS: 1-5, and a light chain variable region that comprises the sequence set forth in any of SEQ ID NOS: 6-11.

In some of any embodiments, the heavy chain variable region comprises the sequence set forth in SEQ ID NO:1, and the light chain variable region comprises the sequence set forth in SEQ ID NO:6. In some of any embodiments, the heavy chain variable region comprises the sequence set forth in SEQ ID NO:1, and in the light chain variable region the sequence set forth in SEQ ID NO:7. In some of any embodiments, the heavy chain variable region comprises the sequence set forth in SEQ ID NO:2, and the light chain variable region comprises the sequence set forth in SEQ ID NO:8. In some of any embodiments, the heavy chain variable region comprises the sequence set forth in SEQ ID NO:3, and the light chain variable region comprises the sequence set forth in SEQ ID NO:9. In some of any embodiments, the heavy chain variable region comprises the sequence set forth in SEQ ID NO: 4, and the light chain variable region the sequence set forth in SEQ ID NO: 10. In some of any embodiments, the heavy chain variable region comprises the sequence set forth in SEQ ID NO: 5, and the light chain variable region comprises the sequence set forth in SEQ ID NO: 11.

In some of any embodiments, any of the antibodies or antigen binding fragments thereof provided herein the sequences disclosed in Table 2.

TABLE 2
VH and VL combinations

| Ab | VH (SEQ ID NO) | VL (SEQ ID NO) |
|---|---|---|
| 1 | DVQLQESGPDLVKPSQSLSLTCTVTGYSIT | DVQITQSPSYLAASPGETITINCRASKSIS |
| | SAYSWHWIRQFPGNKLEWMGYIHYSGST | KYLAWYQEKPGKTNKLLIYSGSTLQSGI |
| | NYNPSLKSRISITRDTSKNQFFLQLNSVTTE | PSRFSGSGSGTDFTLTISSLEPEDFAMYY |
| | DTATYYCARSLWFYAMDYWGQGTSVTVS | CQQHNEYPWTFGGGTKLEIK (SEQ ID |
| | S (SEQ ID NO: 1) | NO: 6) |
| 2 | DVQLQESGPDLVKPSQSLSLTCTVTGYSIT | DIQMNQSPSSLSASLGDTITITCHASQNIN |
| | SAYSWHWIRQFPGNKLEWMGYIHYSGST | VWLNWYQQKPGNIPKLLIYKASNLHTG |
| | NYNPSLKSRISITRDTSKNQFFLQLNSVTTE | VPSRFSGSGSGTGFTLTISSLQPEDIATYY |
| | DTATYYCARSLWFYAMDYWGQGTSVTVS | CQQGQSYPLTFGGGTKLEIK (SEQ ID |
| | S (SEQ ID NO: 1) | NO: 7) |
| 3 | QIQLVQSGPELKKPGETVKISCKASGYTFT | EIVLTQSPTTMAASPGEKITITCSASSSISS |
| | NYGMNWVKQAPGKGLKWMGWINTYIGE | NYLHWYQQKPGFSPKLLIYRTSNLASGV |
| | PTYADDFKGRFAFSLETSASTAYLQINNLK | PARFSGSGSGTSYSLTIGTMEAEDVATY |
| | NEDMGTYFCARGTTFYAMDYWGQGTSVT | YCQQGSSILTFGAGTKLELK (SEQ ID |
| | VSS (SEQ ID NO: 2) | NO: 8) |
| 4 | DVQLQESGPDLVKPSQSLSLTCTVTGYSIT | DVQITQSPSYLAASPGETITIKCRASKSIS |
| | SAYSWHWIRQFPGNKLEWMGYIHYSGST | KYLAWYQEKPGKTNKLLIYSGSTLHSGI |
| | NYNPSLKSRISITRDTSKNQFFLQLSSVTTE | PSRFSGSGSGTDFTLTISSLEPEDFAMYY |
| | DTATYYCARSLWFYAMDYWGQGTSVTVS | CQQHNEYPWTFGGGTKLEIK (SEQ ID |
| | S (SEQ ID NO: 3) | NO: 9) |
| 5 | QIQLVQSGPELKKPGETVKISCKASGYTFT | EIVLTQSPTTMAASPGEKITITCSASSSISS |
| | NYGMNWVKQAPGKGLKWMGWINTYTGE | NYLHWYQQKPGFSPKLLIYRTSNLASGV |
| | PTYADDFKGRFAFSLETSASTAYLQINNLK | PTRFSGSGSGTSYSLTIGTMEAEDVATYY |
| | NEDMATYFCARGDRFYAMDYWGQGTSV | CQQGSSIFTFGSGTKLEIK (SEQ ID |
| | TVSS (SEQ ID NO: 4) | NO: 10) |
| 6 | EVNLVESGGGLVQPGGSLKLSCATSGFTFS | DIVMTQSHKFMSTSVGDRVSITCKASQD |
| | DYYMYWVRQTPEKRLEWVAYISNGGGST | VSTAVAWYQQKPGQSPKLLIYWASTRH |
| | YYPDTVKGRFTISRDNAKNTLYLQMSRLK | TGVPDRFTGSGSGTDYTLTISSVQAEDLA |
| | SEDTAMYYCARLHFYAMDYWGQGTSVT | LYYCQQHYNTPFTFGSGTKLEIK (SEQ ID |
| | VSS (SEQ ID NO: 5) | NO: 11) |

### Fc

An anti-AXL antibody or antigen-binding fragment thereof provided herein may comprise a fragment crystallizable region (Fc region). An Fc region typically forms the tail of an antibody and can interact with certain cell surface receptors and certain components of the complement system. An Fc region may include, for example, two polypeptides, each derived from the second (CH2) and third (CH3) constant domains of an antibody heavy chain.

The amino acid sequence of a wild-type CH2-CH3 portion of an Fc region is provided below (positioning is as in EU index as in Kabat et al. (1992) SEQUENCES OF PROTEINS OF IMMUNOLOGICAL INTEREST, National Institutes of Health Publication No. 91-3242) (SEQ ID NO:63). The CH2 portion is from amino acids 1-110 of SEQ ID NO:63 and the CH3 portion is from amino acids 111-217 of SEQ ID NO:63.

In some embodiments, an Fc region includes one or more modifications (e.g., one or more amino acid substitutions, insertions, or deletions relative to a comparable wild-type Fc region). Antibodies and antigen-binding fragments thereof comprising modified Fc regions (variant agents) typically have altered phenotypes relative to agents comprising wild-type Fc regions. A variant agent phenotype may be expressed as altered serum half-life, altered stability, altered susceptibility to cellular enzymes, or altered effector function (e.g., as assayed in an NK-dependent or macrophage-dependent assay). Fc region modifications that alter effector function may include modifications that increase binding to activating receptors (e.g., FcγRIIA (CD16A)) and reduce binding to inhibitory receptors (e.g., FcγRIIB (CD32B)) (see, e.g., Stavenhagen, J. B. et al. (2007) Cancer Res. 57 (18): 8882-8890). Examples of variants of human IgG1 Fc regions with reduced binding to CD32B and/or increased binding to CD16A contain F243L, R292P, Y300L, V305I and/or P396L substitutions. Amino acid positions correspond to the amino acid numbering of the CH2-CH3 domain provided above.

In some embodiments, an Fc region includes one or more modifications that reduce or abrogate binding of the Fc-to-Fc receptors. Such modifications may include amino acid substitutions at positions 234, 235, 265, and 297 (see e.g., U.S. Pat. No. 5,624,821, which is incorporated by reference herein). Example substitutions include one or more of L234A, L235A, D265A and N297Q. Amino acid positions correspond to the amino acid numbering of the CH2-CH3 domain provided above.

In some embodiments, an Fc region includes one or more modifications that alter (relative to a wild-type Fc region) the Ratio of Affinities of the modified Fc region to an activating FcγR (such as FcγRIIA or FcγRIIIA) relative to an inhibiting FcγR (such as FcγRIIB):

$$Ratio of Affinities = \frac{Wild - Type to Variant Change in Affinity to Fc γ {R}_{Activating}}{Wild - Type to Variant Change in Affinity to Fc γ {R}_{Inhibiting}} ,$$

where a modified Fc region has a Ratio of Affinities greater than 1, an anti-AXL antibody or antigen-binding fragment thereof herein may have particular use in providing a therapeutic or prophylactic treatment of a disease, disorder, or infection, or the amelioration of a symptom thereof, where an enhanced efficacy of effector cell function (e.g., ADCC) mediated by FcγR is desired, e.g., cancer or infectious disease. Where a modified Fc region has a Ratio of Affinities less than 1, an anti-AXL antibody or antigen-binding fragment thereof herein may have particular use in providing a therapeutic or prophylactic treatment of a disease or disorder, or the amelioration of a symptom thereof, where a decreased efficacy of effector cell function mediated by FcγR is desired, e.g., autoimmune or inflammatory disorders. Table 5 lists example single, double, triple, quadruple and quintuple amino acid substitutions having a Ratio of Affinities greater than 1 or less than 1 (see e.g., PCT Publication Nos. WO 04/063351; WO 06/088494; WO 07/024249; WO 06/113665; WO 07/021841; WO 07/106707; WO 2008/140603, each of which is incorporated by reference herein). Amino acid positions correspond to the amino acid numbering of the CH2-CH3 domain provided above.

TABLE 5
Example Single and Multiple Substitutions Listed by Ratio of Affinities

| Ratio | Single | Double | Triple | Quadruple | Quintuple |
|---|---|---|---|---|---|
| >1 | F243L | F243L & | F243L, P247L & | L234F, F243L, | L235V, |
| | D270E | R292P | N421K | R292P & Y300L | F243L, |
| | R292G | F243L & | F243L, R292P & | L235I, F243L, | R292P, |
| | R292P | Y300L | Y300L | R292P & Y300L | Y300L & |
| | | F243L & | F243L, R292P & | L235Q, F243L, | P396L |
| | | P396L | V305I | R292P & Y300L | L235P, |
| | | D270E & | F243L, R292P & | F243L, P247L, | F243L, |
| | | P396L | P396L | D270E & N421K | R292P, |
| | | R292P & | F243L, Y300L & | F243L, R255L, | Y300L & |
| | | Y300L | P396L | D270E & P396L | P396L |
| | | R292P & | P247L, D270E & | F243L, D270E, | F243L, |
| | | V305I | N421K | G316D & R416G | R292P, |
| | | R292P & | R255L, D270E & | F243L, D270E, | V305I, |
| | | P396L | P396L | K392T & P396L | Y300L & |
| | | Y300L & | D270E, G316D & | F243L, D270E, | P396L |
| | | P396L | R416G | P396L & Q419H | |
| | | P396L & | D270E, K392T & | F243L, R292P, | |
| | | Q419H | P396L | Y300L, & P396L | |
| | | | D270E, P396L & | F243L, R292P, | |
| | | | Q419H | V305I & P396L | |
| | | | V284M, R292L | P247L, D270E, | |
| | | | & K370N | Y300L & N421K | |
| | | | R292P, Y300L & | R255L, D270E, | |
| | | | P396L | R292G & P396L | |
| | | | | R255L, D270E, | |
| | | | | Y300L & P396L | |
| | | | | D270E, G316D, | |
| | | | | P396L & R416G | |
| <1 | Y300L | F243L & | F243L, R292P & | | |
| | P396L | P396L | V305I | | |
| | | P247L & | | | |
| | | N421K | | | |
| | | R255L & | | | |
| | | P396L | | | |
| | | R292P & | | | |
| | | V305I | | | |
| | | K392T & | | | |
| | | P396L | | | |
| | | P396L & | | | |
| | | Q419H | | | |

Antibodies and Antigen-Binding Fragments Thereof that Competitively Bind with Anti-AXL Antibodies and Antigen-Binding Fragments Thereof Provided Herein

Provided herein are anti-AXL antibodies and antigen-binding fragments thereof that competitively bind, or are capable of competitively binding, with one or more anti-AXL antibodies and antigen-binding fragments thereof described herein. In particular, provided herein are anti-AXL antibodies and antigen-binding fragments thereof that compete, or are capable of competing, with one or more anti-AXL antibodies and antigen-binding fragments thereof described herein for binding to AXL. Such antibodies and antigen-binding fragments thereof that compete, or are capable of competing, with anti-AXL described herein may be referred to as competitor antibodies and antigen-binding fragments thereof. In certain instances, an antibody or antigen-binding fragment thereof (i.e., competitor antibody or antigen-binding fragment thereof) may be considered to compete for binding to AXL when the competitor binds to the same general region of AXL as an anti-AXL antibody or antigen-binding fragment thereof described herein (i.e., extracellular region or leucine-rich binding domain). In certain instances, an antibody or antigen-binding fragment thereof (i.e., competitor antibody or antigen-binding fragment thereof) may be considered to compete for binding to AXL when the competitor binds to the exact same region of AXL as an anti-AXL antibody or antigen-binding fragment thereof described herein (e.g., exact same peptide (linear epitope) or exact same surface amino acids (conformational epitope)). In certain instances, an antibody or antigen-binding fragment thereof (i.e., competitor antibody or antigen-binding fragment thereof) may be considered capable of competing for binding to AXL when the competitor binds to the same general region of AXL as an anti-AXL antibody or antigen-binding fragment thereof described herein (i.e., extracellular region or leucine-rich binding domain) under suitable assay conditions. In certain instances, an antibody or antigen-binding fragment thereof (i.e., competitor antibody or antigen-binding fragment thereof) may be considered capable of competing for binding to AXL when the competitor binds to the exact same region of AXL as an anti-AXL antibody or antigen-binding fragment thereof described herein (e.g., exact same peptide (linear epitope) or exact same surface amino acids (conformational epitope)) under suitable assay conditions.

In certain instances, an antibody or antigen-binding fragment thereof (i.e., competitor antibody or antigen-binding fragment thereof) may be considered to compete for binding to AXL when the competitor blocks the binding of one or more anti-AXL antibodies or antigen-binding fragments thereof described herein to AXL. In certain instances, an antibody or antigen-binding fragment thereof (i.e., competitor antibody or antigen-binding fragment thereof) may be considered capable of competing for binding to AXL when the competitor blocks the binding of one or more anti-AXL antibodies or antigen-binding fragments thereof described herein to AXL under suitable assay conditions. Whether a competitor blocks the binding of one or more anti-AXL antibodies or antigen-binding fragments thereof described herein to AXL may be determined using a suitable competition assay or blocking assay, such as, for example, a blocking assay as described in the Examples herein. A competitor antibody or antigen-binding fragment thereof may block binding of one or more anti-AXL antibodies or antigen-binding fragments thereof described herein to AXL in a competition or blocking assay by 50% or more, and conversely, one or more anti-AXL antibodies or antigen-binding fragments thereof described herein may block binding of the competitor antibody or antigen-binding fragment thereof to AXL in a competition or blocking assay by about 50% or more. For example, an antibody or antigen-binding fragment thereof (i.e., competitor antibody or antigen-binding fragment thereof) may block binding of one or more anti-AXL antibodies or antigen-binding fragments thereof described herein to AXL in a competition or blocking assay by about 55%, 60%, 65%, 70%, 75%, 80%, 85%, 90%, 95%, or 100%, and conversely, one or more anti-AXL antibodies or antigen-binding fragments thereof described herein may block binding of the competitor antibody or antigen-binding fragment thereof to AXL in a competition or blocking assay by about 55%, 60%, 65%, 70%, 75%, 80%, 85%, 90%, 95%, or 100%.

In certain instances, an antibody or antigen-binding fragment thereof (i.e., competitor antibody or antigen-binding fragment thereof) may be considered to compete for binding to AXL when the competitor binds to AXL with a similar affinity as one or more anti-AXL antibodies or antigen-binding fragments thereof described herein. In certain instances, an antibody or antigen-binding fragment thereof (i.e., competitor antibody or antigen-binding fragment thereof) may be considered capable of competing for binding to AXL when the competitor binds to AXL with a similar affinity as one or more anti-AXL antibodies or antigen-binding fragments thereof described herein under suitable assay conditions. In some embodiments, an antibody or antigen-binding fragment thereof (i.e., competitor antibody or antigen-binding fragment thereof) is considered to compete for binding to AXL when the competitor binds to AXL with an affinity that is at least about 50% of the affinity of one or more anti-AXL antibodies or antigen-binding fragments thereof described herein. For example, an antibody or antigen-binding fragment thereof (i.e., competitor antibody or antigen-binding fragment thereof) may be considered to compete for binding to AXL when the competitor binds to AXL with an affinity that is at least about 55%, 60%, 65%, 70%, 75%, 80%, 85%, 90%, 95%, or 100% of the affinity of one or more anti-AXL antibodies or antigen-binding fragments thereof described herein. A competitor antibody or antigen-binding fragment thereof may comprise any feature described herein for anti-AXL antibodies or antigen-binding fragments thereof.

Also provided herein are anti-AXL antibodies or antigen-binding fragments thereof that bind to, or are capable of binding to, the same epitope as one or more anti-AXL antibodies or antigen-binding fragments thereof described herein. In particular, provided herein are anti-AXL antibodies or antigen-binding fragments thereof that compete with one or more anti-AXL antibodies or antigen-binding fragments thereof described herein for binding to the same epitope on AXL. Such antibodies or antigen-binding fragments thereof that bind the same epitope may be referred to as epitope competitors. In certain instances, an epitope competitor may bind to the exact same region of AXL as an anti-AXL antibody or antigen-binding fragment thereof described herein (e.g., exact same peptide (linear epitope) or exact same surface amino acids (conformational epitope)). In certain instances, an epitope competitor blocks the binding of one or more anti-AXL antibodies or antigen-binding fragments thereof described herein to AXL. An epitope competitor may block binding of one or more anti-AXL antibodies or antigen-binding fragments thereof described herein to AXL in a competition assay by about 50% or more, and conversely, one or more anti-AXL antibodies or antigen-binding fragments thereof described herein may block binding of the epitope competitor to AXL in a competition assay by 50% or more. In certain instances, an epitope competitor binds to AXL with a similar affinity as one or more anti-AXL antibodies or antigen-binding fragments thereof described herein. In some embodiments, an epitope competitor binds to AXL with an affinity that is at least about 50% of the affinity of one or more anti-AXL antibodies or antigen-binding fragments thereof described herein. For example, an epitope competitor may bind to AXL with an affinity that is at least about 55%, 60%, 65%, 70%, 75%, 80%, 85%, 90%, 95%, or 100% of the affinity of one or more anti-AXL antibodies or antigen-binding fragments thereof described herein. An epitope competitor may comprise any feature described herein for anti-AXL antibodies or antigen-binding fragments thereof.

### Antibody Preparation

Methods for generating anti-AXL antibodies and antigen-binding fragments thereof are described in the Examples below. In some embodiments, an anti-AXL antibody or antigen-binding fragment thereof is a humanized antibody or antigen-binding fragment thereof. Humanized anti-AXL antibodies may be prepared, e.g., in a genetically engineered (i.e., transgenic) mouse (e.g., from Medarex) that, when presented with an immunogen, can produce a human antibody that does not necessarily require CDR grafting. These antibodies are fully human (100% human protein sequences) from animals such as mice in which the non-human antibody genes are suppressed and replaced with human antibody gene expression. Antibodies may be generated against AXL when presented to these genetically engineered mice or other animals that can produce human frameworks for the relevant CDRs.

Where a variant of an anti-AXL antibody is generated, the parent antibody is prepared. Example techniques for generating such non-human antibodies and parent antibodies are described in the following sections.

### Antigen Preparation

The antigen for production of antibodies may be, e.g., intact AXL, particularly expressed in cells, or a portion of AXL (e.g., N-terminal domain, C-terminal domain, a cytoplasmic domain, an intra-organelle domain, a transmembrane domain, an extracellular domain, an ectodomain, a TIR domain, a leucine-rich domain, or a AXL fragment comprising a desired epitope). Other forms of antigens useful for generating antibodies will be apparent to those skilled in the art.

### Polyclonal Antibodies

Polyclonal antibodies may be raised in animals (vertebrate or invertebrates, including mammals, birds and fish, including cartilaginous fish) by multiple subcutaneous (sc) or intraperitoneal (ip) injections of a relevant antigen and an adjuvant. It may be useful to conjugate the relevant antigen to a protein or other carrier that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Non-protein carriers (e.g., colloidal gold) also may be used for antibody production.

Animals are immunized against the antigen, immunogenic conjugates, or derivatives by combining, e.g., 100 μg or 5 μg of the protein or conjugate (for rabbits or mice, respectively) with three volumes of Freund's complete adjuvant and injecting the solution intradermally at multiple sites. One month later the animals are boosted with one-fifth to one-tenth of the original amount of peptide or conjugate in Freund's complete adjuvant by subcutaneous injection at multiple sites. Seven to 14 days later the animals are bled and the serum is assayed for antibody titer. Animals are boosted until the titer plateaus. Often, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### Monoclonal Antibodies

Monoclonal antibodies may be made using the hybridoma method first described by Kohler et al., Nature, 256:495 (1975), or may be made by other methods such as recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that may contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. The binding specificity of monoclonal antibodies produced by hybridoma cells may be determined by immunoprecipitation, by an in vitro binding assay, such as radioimmunoassay (RIA) or enzyme-linked immunoabsorbant assay (ELISA), or by flow cytometric analysis of cells expressing the membrane antigen. The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, DMEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). Alternatively, cDNA may be prepared from mRNA and the cDNA then subjected to DNA sequencing. The hybridoma cells serve as a preferred source of such genomic DNA or RNA for cDNA preparation. Once isolated, the DNA may be placed into expression vectors, which are well known in the art, and which are then transfected into host cells such as E coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

### Humanization and Amino Acid Sequence Variants

General methods for humanization of antibodies are described, for example, in U.S. Pat. Nos. 5,861,155, 6,479,284, 6,407,213, 6,639,055, 6,500,931, 5,530,101, 5,585,089, 5,693,761, 5,693,762, 6,180,370, 5,714,350, 6,350,861, 5,777,085, 5,834,597, 5,882,644, 5,932,448, 6,013,256, 6,129,914, 6,210,671, 6,329,511, 5,225,539, 6,548,640, and 5,624,821, each of which is incorporated by reference herein. In certain embodiments, it may be desirable to generate amino acid sequence variants of these humanized antibodies, particularly where these improve the binding affinity or other biological properties of the antibody.

Amino acid sequence variants of the anti-AXL antibody are prepared by introducing appropriate nucleotide changes into the anti-AXL antibody DNA, or by peptide synthesis. Such variants include, for example, deletions from, and/or insertions into and/or substitutions of, residues within the amino acid sequences of the anti-AXL antibodies for the examples herein. Any combination of deletion, insertion, and substitution is made to arrive at the final construct, provided that the final construct possesses the desired characteristics. The amino acid changes also may alter post-translational processes of the humanized or variant anti-AXL antibody, such as changing the number or position of glycosylation sites.

One method for identification of certain residues or regions of the anti-AXL antibody that are preferred locations for mutagenesis is called “alanine scanning mutagenesis,” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with AXL antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed anti-AXL antibody variants are screened for the desired activity. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the antibody fused to an epitope tag. Other insertional variants include the fusion of an enzyme or a polypeptide that increases the serum half-life of the antibody to the N- or C-terminus of the antibody.

Another type of variant is an amino acid substitution variant. These variants have at least one amino acid residue removed from the antibody molecule and a different residue inserted in its place. The sites of greatest interest for substitutional mutagenesis include the hypervariable regions, but FR alterations are also contemplated. Conservative substitutions are preferred, but more substantial changes may be introduced and the products may be screened. Examples of substitutions are listed below:

### Example Amino Acid Residue Substitutions

Ala (A) val; leu; ile; val
Arg (R) lys; gln; asn; lys
Asn (N) gln; his; asp, lys; gln; arg
Asp (D) glu; asn
Cys (C) ser; ala
Gln (Q) asn; glu
Glu (E) asp; gln
Gly (G) ala
His (H) asn; gln; lys; arg
Ile (I) leu; val; met; ala; leu; phe; norleucine
Leu (L) norleucine; ile; val; ile; met; ala; phe
Lys (K) arg; gln; asn
Met (M) leu; phe; ile
Phe (F) leu; val; ile; ala; tyr
Pro (P) ala
Ser(S) thr
Thr (T) ser
Trp (W) tyr; phe
Tyr (Y) trp; phe; thr; ser
Val (V) ile; leu; met; phe; ala; norleucine

Substantial modifications in the biological properties of an antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe

Non-conservative substitutions will entail exchanging a member of one of these classes for another class.

Any cysteine residue not involved in maintaining the proper conformation of the antibody also may be substituted, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino acid substitutions at each site.

The antibody variants thus generated are displayed in the monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine-scanning mutagenesis can be performed to identify hypervariable region residues contributing significantly to antigen-binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one of more carbohydrate moieties found in the antibody, and/or adding one or more glycosylation sites that are not present in the antibody. Glycosylation of antibodies is typically either N-linked and/or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of anti-AXL antibodies herein are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of an anti-AXL antibody.

### Human Antibodies

As an alternative to humanization, human antibodies can be generated. For example, transgenic animals (e.g., mice) may be generated that are capable, upon immunization, of producing a full repertoire of human antibodies in the absence of endogenous immunoglobulin production. For example, a homozygous deletion of the antibody heavy chain joining region (JH) gene in chimeric and germ-line mutant mice results in complete inhibition of endogenous antibody production. Transfer of the human germ-line immunoglobulin gene array into such germ-line mutant mice can result in the production of human antibodies upon antigen challenge (see, e.g., Jakobovits et al., Proc. Natl. Acad. Sci. USA, 90:2551 (1993); Jakobovits et al., Nature, 362:255-258(1993); Bruggermann et al., Year in Immuno., 7:33 (1993); and U.S. Pat. Nos. 5,591,669, 5,589,369 and 5,545,807). Human antibodies also can be derived from phage-display libraries (Hoogenboom et al., J. Mol. Biol., 227:381 (1991); Marks et al., J. Mol. Biol., 222:581-597 (1991); and U.S. Pat. Nos. 5,565,332 and 5,573,905). Human antibodies also may be generated by in vitro activated B cells (see U.S. Pat. Nos. 5,567,610 and 5,229,275).

### Antigen-Binding Antibody Fragments

In certain embodiments, an anti-AXL antibody or antigen-binding fragment thereof is an antibody fragment that retains at least one desired activity, including antigen-binding. Various techniques have been developed for the production of antibody fragments. In some instances, these fragments are derived via proteolytic digestion of intact antibodies (see, e.g., Morimoto et al., Journal of Biochemical and Biophysical Methods 24:107-117 (1992) and Brennan et al., Science 229:81 (1985)). In some instances, these fragments are produced directly by recombinant host cells. For example, Fab′-SH fragments can be directly recovered from E. coli and chemically coupled to form F(ab′)₂ fragments (Carter et al., Bio/Technology 10:163-167 (1992)). In some instances, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to the skilled practitioner.

### Multispecific Antibodies and Antigen-Binding Fragments Thereof

In some embodiments, an anti-AXL antibody or antigen-binding fragment thereof comprises a first binding moiety and a second binding moiety, where the first binding moiety is specifically reactive with a first molecule that is AXL and the second binding moiety is specifically reactive with a second molecule that is a molecular species different from the first molecule. Such an antibody or antigen-binding fragment thereof may comprise a plurality of first binding moieties, a plurality of second binding moieties, or a plurality of first binding moieties and a plurality of second binding moieties. Preferably, the ratio of first binding moieties to second binding moieties is about 1:1, although it may range from about 1000:1 to about 1:1000, where the ratio may be measured in terms of valency.

In those embodiments where the first moiety is an antibody, the second binding moiety may also be an antibody. In some embodiments, the first and second moieties are linked via a linker moiety, which may have two to many hundreds or even thousands of valencies for attachment of first and second binding moieties by one or different chemistries. Examples of bispecific antibodies include those that are reactive against two different epitopes; in some instances, on epitope is a AXL epitope and the second epitope is on an unrelated soluble molecule. In some embodiments, the bispecific antibody is reactive against an epitope on AXL and against an epitope on a different molecule found on the surface of a different cell.

Compositions herein may also comprise a first antibody or antigen-binding fragment thereof and a second antibody or antigen-binding fragment thereof, where the first antibody or antigen-binding fragment thereof comprises a first binding moiety specifically reactive with a first molecule (e.g., AXL) and the second antibody or antigen-binding fragment thereof comprises a second binding moiety specifically reactive with a second molecule that is a molecular species different than the first molecule. The first and/or second antibody or antigen-binding fragment thereof may be an antibody. The ratio of first antibody or antigen-binding fragment thereof to second antibody or antigen-binding fragment thereof may range from about 1,000:1 to 1:1,000, although the preferred ratio is about 1:1. In some embodiments, it may be desirable to generate multispecific (e.g., bispecific) anti-AXL antibodies having binding specificities for at least two different epitopes. Certain bispecific antibodies may bind to two different epitopes of AXL. Bispecific antibodies can be prepared as full-length antibodies or antibody fragments (e.g., F(ab′)₂ bispecific antibodies).

According to one method for making bispecific antibodies, the interface between a pair of antibody molecules can be engineered to maximize the percentage of heterodimers that are recovered from recombinant cell culture. The preferred interface comprises at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end products such as homodimers (see e.g., WO96/27011 published Sep. 6, 1996).

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,678,980, along with a number of cross-linking techniques.

Any suitable technique may be used for generating bispecific antibodies from antibody fragments. For example, bispecific antibodies can be prepared using chemical linkage. In certain methods, intact antibodies are proteolytically cleaved to generate F(ab′)₂ fragments (see e.g., Brennan et al., Science 229:81 (1985), which is incorporated by reference herein). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-thiol derivative to form the bispecific antibody. In yet a further embodiment, Fab′-SH fragments directly recovered from E. coli can be chemically coupled in vitro to form bispecific antibodies (see e.g., Shalaby et al., J. Exp. Med. 175:217-225 (1992), which is incorporated by reference herein).

Any suitable technique for making and isolating bispecific antibody fragments directly from recombinant cell culture may be used. For example, bispecific antibodies have been produced using leucine zippers (see e.g., Kostelny et al., J. Immunol. 148(5): 1547-1553 (1992), which is incorporated by reference herein). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the VH and VL domains of one fragment are forced to pair with VL and VH domains of another fragment, thereby forming two antigen-binding sites. Another strategy for making bispecific antibody fragments by the use of single chain Fv (scFv) dimers (see e.g., Gruber et al., J. Immunol. 152:5368 (1994), which is incorporated by reference herein). In certain instances, a bispecific antibody may be a “linear antibody” produced as described in Zapata et al. Protein Eng. 8(10):1057-1062 (1995), which is incorporated by reference herein.

Antibodies with two valencies or more are contemplated herein. An antibody (or polymer or polypeptide) herein comprising one or more binding sites per arm or fragment thereof will be referred to herein as a “multivalent” antibody. For example, a “bivalent” antibody herein comprises two binding sites per Fab or fragment thereof whereas a “trivalent” polypeptide herein comprises three binding sites per Fab or fragment thereof. In a multivalent polymer herein, the two or more binding sites per Fab may be binding to the same or different antigens. For example, the two or more binding sites in a multivalent polypeptide herein may be directed against the same antigen, for example against the same parts or epitopes of said antigen or against two or more same or different parts or epitopes of said antigen; and/or may be directed against different antigens; or a combination thereof. Thus, a bivalent polypeptide herein, for example, may comprise two identical binding sites, may comprise a first binding sites directed against a first part or epitope of an antigen and a second binding site directed against the same part or epitope of said antigen or against another part or epitope of said antigen; or may comprise a first binding sites directed against a first part or epitope of an antigen and a second binding site directed against a different antigen. However, as will be clear from the description hereinabove, the technology herein is not limited thereto, in the sense that a multivalent polypeptide herein may comprise any number of binding sites directed against the same or different antigens. In one embodiment the multivalent polypeptide comprises at least two ligand binding elements, one of which contains one or more CDR peptide sequences shown herein. In another embodiment the multivalent polypeptide comprises three ligand binding sites, each independently selected from the CDR sequences disclosed herein.

In certain embodiments, at least one of the ligand binding elements binds AXL. In one embodiment, at least one of the ligand binding elements binds another target. In one embodiment, there are up to 10,000 binding elements in a multivalent binding molecule, and the ligand binding elements may be linked to a scaffold.

An antibody (or polymer or polypeptide) herein that contains at least two binding sites per Fab or fragment thereof, in which at least one binding site is directed against a first antigen and a second binding site directed against a second antigen different from the first antigen, may also be referred to as “multispecific.” Thus, a “bispecific” polymer comprises at least one site directed against a first antigen and at least one second site directed against a second antigen, whereas a “trispecific” is a polymer that comprises at least one binding site directed against a first antigen, at least one further binding site directed against a second antigen, and at least one further binding site directed against a third antigen; and the like. Accordingly, in their simplest form, a bispecific polypeptide herein is a bivalent polypeptide (per Fab) of the technology provided herein. However, as will be clear from the description hereinabove, the technology herein is not limited thereto, in the sense that a multispecific polypeptide herein may comprise any number of binding sites directed against two or more different antigens.

### Other Modifications

Other modifications of an anti-AXL antibody or antigen-binding fragment thereof are contemplated. For example, technology herein also pertains to immunoconjugates comprising an antibody described herein (e.g., an anti-AXL antibody) conjugated to a cytotoxic agent such as a toxin (e.g., an enzymatically active toxin of bacterial, fungal, plant or animal origin, or fragments thereof), or a radioactive isotope (for example, a radioconjugate), or a cytotoxic drug. Such conjugates are sometimes referred to as “antibody-drug conjugates” or “ADC.” Conjugates are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

An anti-AXL antibody or antigen-binding fragment thereof disclosed herein may be formulated as immunoliposomes. Liposomes containing an antibody are prepared by methods know in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. For example, liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al., J. Biol. Chem. 257:286-288 (1982) via a disulfide interchange reaction. Another active ingredient is optionally contained within the liposome.

Enzymes or other polypeptides can be covalently bound to an anti-AXL antibody or antigen-binding fragment thereof by techniques well known in the art such as the use of the heterobifunctional cross-linking reagents discussed above. In some embodiments, fusion proteins comprising at least the antigen-binding region of an antibody provided herein linked to at least a functionally active portion of an enzyme can be constructed using recombinant DNA techniques well known in the art (see, e.g., Neuberger et al., Nature 312:604-608 (1984)).

In certain embodiments, it may be desirable to use an antibody fragment, rather than an intact antibody, to increase penetration of target tissues and cells, for example. In such instances, it may be desirable to modify the antibody fragment to increase its serum half-life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis; see, e.g., WO96/32478 published Oct. 17, 1996).

Covalent modifications of an anti-AXL antibody or antigen-binding fragment thereof are also included within the scope of this technology. For example, modifications may be made by chemical synthesis or by enzymatic or chemical cleavage of an anti-AXL antibody. Other types of covalent modifications of an antibody are introduced into the molecule by reacting targeted amino acid residues of the antibody with an organic derivatizing agent capable of reacting with selected side chains or the N- or C-terminal residues. Example covalent modifications of polypeptides are described in U.S. Pat. No. 5,534,615, specifically incorporated herein by reference. A preferred type of covalent modification of the antibody comprises linking the antibody to one of a variety of non-proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

In some embodiments, any of the antibodies or antigen fragments thereof disclosed herein are conjugated or hybridized to an oligonucleotide label. In some embodiments, the oligonucleotide label includes a sample barcode sequence, a binding site for a primer and an anchor. In some embodiments, the oligonucleotide label can be conjugated or hybridized to any of the detectable markers or labels disclosed herein. In some embodiments, the oligonucleotide label is a polymeric sequence. In some embodiments, the terms “oligonucleotide” and “polynucleotide” are used interchangeably to refer to a single-stranded multimer of nucleotides from about 2 to about 500 nucleotides in length. In some embodiments, any of the oligonucleotide labels described herein can be synthetic, made enzymatically (e.g., via polymerization), or using a “split-pool” method. In some embodiments, any of the oligonucleotide labels described herein can include ribonucleotide monomers (i.e., can be oligoribonucleotides) and/or deoxyribonucleotide monomers (i.e., oligodeoxyribonucleotides). In some embodiments, any of the oligonucleotide labels described herein can include a combination of both deoxyribonucleotide monomers and ribonucleotide monomers in the oligonucleotide (e.g., random or ordered combination of deoxyribonucleotide monomers and ribonucleotide monomers). In some embodiments, the oligonucleotide label can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length. In some embodiments, any of the oligonucleotide labels described herein can include one or more functional moieties that are attached (e.g., covalently or non-covalently) to another structure. In some embodiments, any of the oligonucleotide labels described herein can include one or more detectable labels (e.g., a radioisotope or fluorophore). In some embodiments, the anchor is a defined polymer, e.g., a polynucleotide or oligonucleotide sequence, which is designed to hybridize to a complementary oligonucleotide sequence. In some embodiments, the anchor is designed for the purpose of generating a double stranded construct oligonucleotide sequence. In some embodiments, the anchor is positioned at the 3′ end of the construct oligonucleotide sequence. In other embodiments, the anchor is positioned at the 5′ end of the construct oligonucleotide sequence. Each anchor is specific for its intended complementary sequence.

In some embodiments, the sample barcode sequence is a polymer, e.g., a polynucleotide, which when it is a functional element, is specific for a single ligand. In some embodiments, the sample barcode sequence can be used for identifying a particular cell or substrate, e.g., Drop-seq microbead. In some embodiments, the sample barcode sequence can be formed of a defined sequence of DNA, RNA, modified bases or combinations of these bases, as well as any other polymer defined above. In some embodiments, the sample barcode sequence is about 2 to 4 monomeric components, e.g., nucleotide bases, in length. In other embodiments, the barcode is at least about 1 to 100 monomeric components, e.g., nucleotides, in length. Thus in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In some embodiments, the sample barcode sequence is a particular barcode that can be unique relative to other barcodes.

In some of any embodiments, the sample barcode sequences can have a variety of different formats. For example, sample barcode sequences can include polynucleotide barcodes, random nucleic acid and/or amino acid sequences, and synthetic nucleic acid and/or amino acid sequences. A sample barcode sequence can be attached to an analyte or to another moiety or structure in a reversible or irreversible manner. A sample barcode sequences can be added to, for example, a fragment of a deoxyribonucleic acid (DNA) or ribonucleic acid (RNA) sample before or during sequencing of the sample. Sample barcode sequences can allow for identification and/or quantification of individual sequencing-reads (e.g., a barcode can be or can include a unique molecular identifier or “UMI”).

Sample barcode sequences can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

In some embodiments, the binding site for a primer is a functional component of the oligonucleotide which itself is an oligonucleotide or polynucleotide sequence that provides an annealing site for amplification of the oligonucleotide. The binding site for a primer can be formed of polymers of DNA, RNA, PNA, modified bases or combinations of these bases, or polyamides, etc. In some embodiments, the binding site for a primer is about 10 of such monomeric components, e.g., nucleotide bases, in length. In other embodiments, the binding site for a primer is at least about 5 to 100 monomeric components, e.g., nucleotides, in length. Thus in various embodiments, the binding site for a primer is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In certain embodiments, the binding site for a primer can be a generic sequence suitable as an annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependent amplification (I), ramification amplification method and RNA-seq. Methods for conjugating or hybridizing an oligonucleotide label can be performed in a manner set forth in WO/2018/144813, WO/2016/018960, WO/2018/089438, WO/2014/182528, WO/2018/026873, WO/2021/188838.

### Nucleic Acids, Vectors, Host Cells, and Recombinant Methods

Technology described herein also provides isolated nucleic acids encoding an anti-AXL antibody or antigen-binding fragment thereof, vectors and host cells comprising the nucleic acid, and recombinant techniques for the production of the antibody or antigen-binding fragment thereof. A nucleic acid herein may include one or more subsequences, each referred to as a polynucleotide.

Provided herein are nucleic acids (e.g., isolated nucleic acids) comprising a nucleotide sequence that encodes an anti-AXL antibody or antigen-binding fragment thereof. In some embodiments, a nucleic acid encodes an immunoglobulin heavy chain variable domain of any of the anti-AXL antibodies or antigen-binding fragments thereof provided herein. In some embodiments, a nucleic acid encodes an immunoglobulin light chain variable domain of any of the anti-AXL antibodies or antigen-binding fragments thereof provided herein. In some embodiments, a nucleic acid encodes an immunoglobulin heavy chain variable domain and an immunoglobulin light chain variable domain of an anti-AXL antibody or antigen-binding fragment thereof provided herein. In some embodiments, a nucleic acid comprises a nucleotide sequence that encodes an amino acid sequence of any one of SEQ ID NOS: 12-23. A nucleic acid may comprise a nucleotide sequence that encodes an immunoglobulin heavy chain variable domain amino acid sequence of any one of SEQ ID NOS: 12-17. A nucleic acid may comprise a nucleotide sequence that encodes an immunoglobulin light chain variable domain amino acid sequence of any one of SEQ ID NOS: 18-23.

Provided herein is an isolated nucleic acid comprising a nucleotide sequence that encodes the immunoglobulin heavy chain variable domain of any of the anti-AXL antibodies or antigen-binding fragments thereof described herein, in which the nucleotide sequence has at least 90% (e.g., 90%, 91%, 92%, 93%, 94%, 95%, 96%, 97%, 98%, 99%, or 100%) identity to any of SEQ ID NOS: 12-17.

Provided herein is an isolated nucleic acid comprising a nucleotide sequence that encodes the immunoglobulin light chain variable domain of any of the anti-AXL antibodies or antigen-binding fragments thereof described herein, in which the nucleotide sequence has at least 90% (e.g., 90%, 91%, 92%, 93%, 94%, 95%, 96%, 97%, 98%, 99%, or 100%) identity to any of SEQ ID NOS: 18-23.

Provided herein is an isolated nucleic acid comprising a nucleotide sequence that encodes the immunoglobulin heavy chain variable domain and the immunoglobulin light chain variable domain of any of the anti-AXL antibodies or antigen-binding fragments thereof described herein, in which the nucleotide sequence encoding the immunoglobulin heavy chain variable domain has at least 90% (e.g., 90%, 91%, 92%, 93%, 94%, 95%, 96%, 97%, 98%, 99%, or 100%) identity to any of SEQ ID NOS: 12-17 and the nucleotide sequence encoding he immunoglobulin light chain variable domain has at least 90% (e.g., 90%, 91%, 92%, 93%, 94%, 95%, 96%, 97%, 98%, 99%, or 100%) identity to any of SEQ ID NOS: 18-23.

Provided below in Table 3 are examples of VH nucleotide (NT) sequences related to the antibodies described herein.

TABLE 3
Nucleic acid sequences of VH

| SEQ ID | | |
|---|---|---|
| NO | SEQUENCE | AB |
| 12 | ATGAGAGTGCTGATTCTTTTGTGCCTGTTCACAGCCTTTCCTGGTATCCTGTCTGATGT | AB1 |
| | GCAGCTTCAGGAGTCAGGACCTGACCTGGTGAAACCTTCTCAGTCACTTTCACTCACC | |
| | TGCACTGTCACTGGCTACTCCATCACCAGTGCTTATAGCTGGCACTGGATCCGGCAGT | |
| | TTCCAGGAAACAAACTGGAATGGATGGGCTACATACACTACAGTGGTAGCACTAACT | |
| | ACAACCCATCTCTCAAAAGTCGAATCTCTATCACTCGAGACACATCCAAGAACCAGTTC | |
| | TTCCTGCAGTTGAATTCTGTGACTACTGAGGACACAGCCACATATTACTGTGCAAGAA | |
| | GCCTATGGTTCTATGCTATGGACTACTGGGGTCAAGGAACCTCAGTCACCGTCTCCTC | |
| | A | |
| 13 | ATGAGAGTGCTGATTCTTTTGTGCCTGTTCACAGCCTTTCCTGGTATCCTGTCTGATGT | AB2 |
| | GCAGCTTCAGGAGTCAGGACCTGACCTGGTGAAACCTTCTCAGTCACTTTCACTCACC | |
| | TGCACTGTCACTGGCTACTCCATCACCAGTGCTTATAGCTGGCACTGGATCCGGCAGT | |
| | TTCCAGGAAACAAACTGGAATGGATGGGCTACATACACTACAGTGGTAGCACTAACT | |
| | ACAACCCATCTCTCAAAAGTCGAATCTCTATCACTCGAGACACATCCAAGAACCAGTTC | |
| | TTCCTGCAGTTGAATTCTGTGACTACTGAGGACACAGCCACATATTACTGTGCAAGAA | |
| | GCCTATGGTTCTATGCTATGGACTACTGGGGTCAAGGAACCTCAGTCACCGTCTCCTC | |
| | A | |
| 14 | ATGGATTGGCTGTGGAACTTGCTATTCCTGATGGCAGCTGCCCAAAGTGCCCAAGCAC | AB3 |
| | AGATCCAGTTGGTGCAGTCTGGACCTGAGCTGAAGAAGCCTGGAGAGACAGTCAAG | |
| | ATCTCCTGCAAGGCTTCTGGATATACCTTCACAAACTATGGAATGAACTGGGTGAAGC | |
| | AGGCTCCAGGAAAGGGTTTGAAGTGGATGGGCTGGATAAACACCTACATTGGAGAG | |
| | CCAACATATGCTGATGACTTCAAGGGACGGTTTGCCTTCTCTTTGGAAACCTCTGCCA | |
| | GCACTGCCTATTTGCAGATCAACAACCTCAAAAATGAGGACATGGGTACATATTTCTG | |
| | TGCAAGGGGGACTACGTTCTATGCTATGGACTACTGGGGTCAAGGAACCTCAGTCAC | |
| | CGTCTCCTCA | |
| 15 | ATGAGAGTGCTGATTCTTTTGTGCCTGTTCACAGCCTTTCCTGGTATCCTGTCTGATGT | AB4 |
| | GCAGCTTCAGGAGTCAGGACCTGACCTGGTGAAACCTTCTCAGTCACTTTCACTCACC | |
| | TGCACTGTCACTGGCTACTCCATCACCAGTGCTTATAGCTGGCACTGGATCCGGCAGT | |
| | TTCCAGGAAACAAACTGGAATGGATGGGCTACATACACTACAGTGGTAGCACTAACT | |
| | ACAACCCATCTCTCAAAAGTCGAATCTCTATCACTCGAGACACATCCAAGAACCAGTTC | |
| | TTCCTGCAGTTGAGTTCTGTGACTACTGAGGACACAGCCACATATTACTGTGCAAGAA | |
| | GCCTATGGTTCTATGCTATGGACTACTGGGGTCAAGGAACCTCAGTCACCGTCTCCTC | |
| | A | |
| 16 | ATGGATTGGCTGTGGAACTTGCTATTCCTGATGGCAGCTGCCCAAAGTGCCCAAGCAC | AB5 |
| | AGATCCAGTTGGTGCAGTCTGGACCTGAGCTGAAGAAGCCTGGAGAGACAGTCAAG | |
| | ATCTCCTGCAAGGCTTCTGGGTATACCTTCACAAACTATGGAATGAACTGGGTGAAGC | |
| | AGGCTCCAGGAAAGGGTTTAAAGTGGATGGGCTGGATAAACACCTACACTGGAGAG | |
| | CCAACATATGCTGATGACTTCAAGGGACGGTTTGCCTTCTCTTTGGAAACCTCTGCCA | |
| | GCACTGCCTATTTGCAGATCAACAACCTCAAAAATGAGGACATGGCTACATATTTCTG | |
| | TGCAAGAGGTGATAGGTTCTATGCTATGGACTACTGGGGTCAAGGAACCTCAGTCAC | |
| | CGTCTCCTCA | |
| 17 | ATGAACTTGGGGCTCAGCTTGATTTTCCTTGTCCTTGTTTTAAAAGGTGTCCAGTGTGA | AB6 |
| | AGTGAACCTGGTGGAGTCTGGGGGAGGCTTAGTGCAGCCTGGAGGGTCCCTGAAAC | |
| | TCTCCTGTGCAACCTCTGGATTCACTTTCAGTGACTATTACATGTATTGGGTTCGCCAG | |
| | ACTCCAGAGAAGAGGCTGGAGTGGGTCGCATACATTAGTAATGGTGGTGGTAGCAC | |
| | CTATTATCCAGACACTGTAAAGGGCCGATTCACCATCTCCAGAGACAATGCCAAGAAC | |
| | ACCCTGTACCTGCAAATGAGCCGTCTGAAGTCTGAGGACACAGCCATGTATTACTGTG | |
| | CAAGACTTCATTTCTATGCTATGGACTACTGGGGTCAAGGAACCTCAGTCACCGTCTC | |
| | CTCA | |

Provided below in Table 4 are examples of VL nucleotide (NT) sequences related to the antibodies described herein.

TABLE 4
Nucleic acid sequences of VL

| SEQ ID | | |
|---|---|---|
| NO | SEQUENCE | AB |
| 18 | ATGAGGTTCCAGGTTCAGGTTCTGGGGCTCCTTCTGCTCTGGATATCAGGTGCCCAGT | AB1 |
| | GTGATGTCCAGATAACCCAGTCTCCATCTTATCTTGCTGCATCTCCTGGAGAAACCATT | |
| | ACTATTAATTGCAGGGCAAGTAAGAGCATTAGCAAATATTTAGCCTGGTATCAAGAG | |
| | AAACCTGGGAAAACTAATAAGCTTCTTATCTACTCTGGATCCACTTTGCAATCTGGAAT | |
| | TCCATCAAGGTTCAGTGGCAGTGGATCTGGTACAGATTTCACTCTCACCATCAGTAGC | |
| | CTGGAGCCTGAAGATTTTGCAATGTATTACTGTCAACAGCATAATGAATACCCGTGGA | |
| | CGTTCGGTGGAGGCACCAAGCTGGAAATCAAA | |
| 19 | ATGAGGGTCCTTGCTGAGCTCCTGGGGCTGCTGCTGTTCTGCTTTTTAGGTGTGAGAT | AB2 |
| | GTGACATCCAGATGAACCAGTCTCCATCCAGTCTGTCTGCATCCCTTGGAGACACAAT | |
| | TACCATCACTTGCCATGCCAGTCAGAACATTAATGTTTGGTTAAACTGGTACCAGCAG | |
| | AAACCAGGAAATATTCCTAAACTATTGATCTATAAGGCTTCCAACTTGCACACAGGCG | |
| | TCCCATCAAGGTTTAGTGGCAGTGGATCTGGAACAGGTTTCACATTAACCATCAGCAG | |
| | CCTGCAGCCTGAAGACATTGCCACTTACTACTGTCAACAGGGTCAAAGTTATCCTCTG | |
| | ACGTTCGGTGGAGGCACCAAGCTGGAAATCAAA | |
| 20 | ATGCAGATTATCAGCTTGCTGCTAATCAGTGTCACAGTCATAGTGTCTAATGGAGAAA | AB3 |
| | TTGTGCTCACCCAGTCTCCAACCACCATGGCTGCATCTCCCGGGGAGAAGATCACTAT | |
| | CACCTGCAGTGCCAGCTCAAGTATAAGTTCCAATTACTTGCATTGGTATCAGCAGAAG | |
| | CCAGGATTCTCCCCTAAACTCTTGATTTATAGGACATCCAATCTGGCTTCTGGAGTCCC | |
| | AGCTCGCTTCAGTGGCAGTGGGTCTGGGACCTCTTACTCTCTCACAATTGGCACCATG | |
| | GAGGCTGAAGATGTTGCCACTTACTACTGCCAGCAGGGTAGTAGTATACTCACGTTCG | |
| | GTGCTGGGACCAAGCTGGAGCTGAAA | |
| 21 | ATGAGGTTCCAGGTTCAGGTTCTGGGGCTCCTTCTGCTCTGGATATCAGGTGCCCAGT | AB4 |
| | GTGATGTCCAGATAACCCAGTCTCCATCCTATCTTGCTGCATCTCCTGGAGAAACCATT | |
| | ACTATTAAATGCAGGGCAAGTAAGAGCATTAGTAAATATTTAGCCTGGTATCAAGAG | |
| | AAACCTGGGAAAACTAATAAGCTTCTTATCTACTCTGGATCCACTTTGCATTCTGGAAT | |
| | TCCATCAAGGTTCAGTGGCAGTGGATCTGGTACAGATTTCACTCTCACCATCAGTAGC | |
| | CTGGAGCCTGAAGATTTTGCAATGTATTACTGTCAACAGCATAATGAATACCCGTGGA | |
| | CGTTCGGTGGAGGCACCAAGCTGGAAATCAAA | |
| 22 | ATGCAGATTATCAGCTTGCTGCTAATCAGTGTCACAGTCATAGTGTCTAATGGAGAAA | AB5 |
| | TTGTGCTCACCCAGTCTCCAACCACCATGGCTGCATCTCCCGGGGAGAAGATCACTAT | |
| | CACCTGCAGTGCCAGCTCAAGTATAAGTTCCAATTACTTGCATTGGTATCAGCAGAAG | |
| | CCAGGATTCTCCCCTAAACTCTTGATTTATAGGACATCCAATCTGGCTTCTGGAGTCCC | |
| | AACTCGCTTCAGTGGCAGTGGGTCTGGGACCTCTTACTCTCTCACAATTGGCACCATG | |
| | GAGGCTGAAGATGTTGCCACTTACTACTGCCAGCAGGGTAGTAGTATATTCACGTTCG | |
| | GCTCGGGGACAAAGTTGGAAATAAAA | |
| 23 | ATGGAGTCACAGATTCAGGCATTTGTATTCGTGTTTCTCTGGTTGTCTGGTGTTGACG | AB6 |
| | GAGACATTGTGATGACCCAGTCTCACAAATTCATGTCCACATCAGTAGGAGACAGGG | |
| | TCAGCATCACCTGCAAGGCCAGTCAGGATGTGAGTACTGCTGTAGCCTGGTATCAAC | |
| | AAAAACCAGGGCAATCTCCTAAACTACTGATTTACTGGGCATCCACCCGGCACACTGG | |
| | AGTCCCTGATCGCTTCACAGGCAGTGGATCTGGGACAGATTATACTCTCACCATCAGC | |
| | AGTGTGCAGGCTGAAGACCTGGCACTTTATTACTGTCAGCAACATTATAACACTCCAT | |
| | TCACGTTCGGCTCGGGGACAAAGTTGGAAATAAAA | |

In some embodiments, any of the nucleic acids provided herein comprises a signal sequence. In some embodiments, any of the nucleic acids described herein does not comprise a signal sequence.

For recombinant production of an anti-AXL antibody or antigen-binding fragment thereof, a nucleic acid encoding the anti-AXL antibody or antigen-binding fragment thereof may be isolated and inserted into a replicable vector for further cloning (amplification of the DNA) or for expression. In certain instances, an anti-AXL antibody or antigen-binding fragment thereof may be produced by homologous recombination, e.g., as described in U.S. Pat. No. 5,204,244, specifically incorporated herein by reference. DNA encoding an anti-AXL antibody or antigen-binding fragment thereof can be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, and origin of replication, one or more marker genes, an enhancer element, a promoter, and a transcription termination sequence, e.g., as described in U.S. Pat. No. 5,534,615 issued Jul. 9, 1996 and specifically incorporated herein by reference.

Suitable host cells for cloning or expressing DNA in vectors herein are prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescens, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X₁₇₇₆ (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-AXL antibody or antigen-binding fragment thereof-encoding vectors. Saccharomyces cerevisiae, or common baker's yeast, is the most commonly used among lower eukaryotic host microorganisms. A number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of an anti-AXL antibody or antigen-binding fragment thereof (e.g., a glycosylated anti-AXL antibody or antigen-binding fragment thereof) are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk moth) have been identified. A variety of viral strains for transfection are publicly available, e.g., the L-1 variant of Autographa californica NPV and the Bm-5 strain of Bombyx mori NPV, and such viruses may be used as the virus herein according to the present technology, particularly for transfection of Spodoptera frugiperda cells. Plant cell cultures of cotton, corn, potato, soybean, petunia, tomato, and tobacco can also be utilized as hosts.

Suitable host cells for the expression of an anti-AXL antibody or antigen-binding fragment thereof also may include vertebrate cells (e.g., mammalian cells). Vertebrate cells may be propagated in culture (tissue culture). Examples of useful mammalian host cell lines include monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells may be transformed with the above-described expression or cloning vectors for antibody production and cultured in conventional nutrient media modified as appropriate for inducing promoters, selecting transformants, or amplifying the genes encoding the desired sequences.

Host cells used to produce an antibody or antigen-binding fragment thereof herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, an antibody or antigen-binding fragment thereof can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody or antigen-binding fragment thereof composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody or antigen-binding fragment thereof of interest and contaminants may be subjected to low pH hydrophobic interaction chromatography using an elution buffer at a pH between about 2.5-4.5, and may be performed at low salt concentrations (e.g., from about 0-0.25M salt).

### Pharmaceutical Formulations, Dosing, and Routes of Administration

The present technology provides anti-AXL antibodies or antigen-binding fragments thereof and related compositions, which may be useful for elimination of AXL-expressing cells from the body, for example, and for identification and quantification of the number of AXL-expressing cells in tissue samples, for example.

Therapeutic methods and compositions of the present technology may be referred to as “AXL-based” to indicate that these therapies can change the relative or absolute numbers of undesirable or toxic AXL-expressing cells such as lymphomas or autoimmune B lymphocytes.

One way to control the amount of undesirable AXL-expressing cells in a patient is by providing a composition that comprises one or more anti-AXL antibodies to cause cytotoxic activity towards the AXL-expressing cells, for example.

Anti-AXL antibodies or antigen-binding fragments thereof may be formulated in a pharmaceutical composition that is useful for a variety of purposes, including the treatment of diseases, disorders, or physical trauma. Pharmaceutical compositions comprising one or more anti-AXL antibodies or antigen-binding fragments thereof herein may be used to administer pharmaceutical compositions herein to a patient in need thereof, and according to one embodiment of the technology, kits are provided that include such devices. Such devices and kits may be designed for routine administration, including self-administration, of the pharmaceutical compositions herein.

Therapeutic formulations of an antibody or antigen-binding fragment thereof may be prepared for storage by mixing the antibody or antigen-binding fragment thereof having the desired degree of purity with optional physiologically acceptable carriers, excipients or stabilizers (Remington's Pharmaceutical Sciences 16^{th} edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

Formulations herein may also contain more than one active compound as necessary for the particular indication being treated, preferably those with complementary activities that do not adversely affect each other. Such molecules are suitably present in combination in amounts that are effective for the purpose intended.

Active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences 16^{th} edition, Osol, A. Ed. (1980).

Formulations for in vivo administration generally are sterile. This may be accomplished for instance by filtration through sterile filtration membranes, for example.

Sustained-release preparations may be prepared. Suitable examples of sustained-release preparations include semipermeable matrices of solid hydrophobic polymers containing the antibody or antigen-binding fragment thereof, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as such as ethylene-vinyl acetate and lactic acid-glycolic acid enable release of molecules for over 100 days, certain hydrogels release proteins for shorter time periods. When encapsulated antibodies or antigen-binding fragments thereof remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

For therapeutic applications, anti-AXL antibodies or antigen-binding fragments thereof, provided herein are administered to a mammal, e.g., a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, oral, topical, or inhalation routes.

For the prevention or treatment of disease, the appropriate dosage of an antibody or antigen-binding fragment thereof will depend on the type of disease to be treated, as defined above, the severity and course of the disease, whether the antibody is administered for preventative or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to about 50 mg/kg (e.g., 0.1-20 mg/kg) of antibody may be an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine AXL levels in bodily fluids or tissues may be used in order to optimize patient exposure to the therapeutic antibody.

In some embodiments, a composition comprising an anti-AXL antibody or antigen-binding fragment thereof herein (e.g., a mAb that interferes with AXL activity) is administered as a monotherapy, and in some embodiments, the composition comprising the anti-AXL antibody or antigen-binding fragment thereof is administered as part of a combination therapy. In some cases, the effectiveness of the antibody or antigen-binding fragment thereof is preventing or treating disease may be improved by administering the antibody or antigen-binding fragment thereof serially or in combination with another antibody or antigen-binding fragment thereof that is effective for those purposes, such as a chemotherapeutic drug for treatment of cancer or a microbial infection. In other cases, the anti-AXL antibody or antigen-binding fragment thereof may serve to enhance or sensitize cells to chemotherapeutic treatment, thus permitting efficacy at lower doses and with lower toxicity. Certain combination therapies include, in addition to administration of the composition comprising an antibody or antigen-binding fragment thereof that reduces the number of AXL-expressing cells, delivering a second therapeutic regimen selected from the group consisting of administration of a chemotherapeutic agent, radiation therapy, surgery, and a combination of any of the foregoing.

Such other agents may be present in the composition being administered or may be administered separately. Also, the anti-AXL antibody or antigen-binding fragment thereof may be suitably administered serially or in combination with the other agent or modality, e.g., chemotherapeutic drug or radiation for treatment of cancer, infection, and the like, or an immunosuppressive drug.

### Research and Diagnostic, Including Clinical Diagnostic, Uses for Anti-AXL Antibodies and Antigen-Binding Fragments Thereof Provided Herein

Provided herein are diagnostic reagents comprising an anti-AXL antibody or antigen-binding fragment thereof described herein. For example, anti-AXL antibodies or antigen-binding fragments thereof provided herein may be used to detect and/or purify AXL, e.g., from bodily fluid(s) or expressed on cells in bodily fluids or tissues. Also provided herein are methods for detecting AXL. For example, a method may comprise contacting a sample (e.g., a biological sample known or suspected to contain AXL) with an anti-AXL antibody or antigen-binding fragment thereof provided herein, and, if the sample contains AXL, detecting AXL:anti-AXL complexes. Also provided herein are reagents comprising and anti-AXL antibody or antigen-binding fragment thereof described herein and methods for detecting AXL for research purposes.

Anti-AXL antibodies, for example, may be useful in diagnostic assays for AXL, e.g., detecting its presence in specific cells, tissues, or bodily fluids. Such diagnostic methods may be useful in diagnosis, e.g., of a hyperproliferative disease or disorder. Thus clinical diagnostic uses as well as research uses are comprehended herein.

In some embodiments, an anti-AXL antibody or antigen-binding fragment thereof comprises a detectable marker or label. In some embodiments, an anti-AXL antibody or antigen-binding fragment thereof is conjugated to a detectable marker or label. For example, for research and diagnostic applications, an anti-AXL antibody or antigen-binding fragment thereof may be labeled with a detectable moiety. Numerous labels are available which are generally grouped into the following categories:
(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.
(b) Fluorescent labels such as rare earth chelates (europium chelates) or fluorescein and its derivatives, rhodamine and its derivatives, dansyl, Lissamine, phycoerythrin, Texas Red and Brilliant Violet™ are available. The fluorescent labels can be conjugated to the antibody using the techniques disclosed in Current Protocols in Immunology, supra, for example. Fluorescence can be quantified using a flow cytometer, imaging microscope or fluorimeter.
(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemilluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:
(i) Horseradish peroxidase (HRP) with hydrogen peroxidase as a substrate, where the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations may be used (e.g., U.S. Pat. Nos. 4,275,149 and 4,318,980, each of which is incorporated by reference herein).

In certain instances, the label is indirectly conjugated with the antibody or antigen-binding fragment thereof. The skilled artisan will be aware of various techniques for achieving this. For example, an antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In some embodiments, and anti-AXL antibody or antigen-binding fragment thereof need not be labeled, and the presence thereof can be detected, e.g., using a labeled antibody which binds to an anti-AXL antibody.

In some embodiments, an anti-AXL antibody or antigen-binding fragment thereof herein is immobilized on a solid support or substrate. In some embodiments, an anti-AXL antibody or antigen-binding fragment thereof herein is non-diffusively immobilized on a solid support (e.g., the anti-AXL antibody or antigen-binding fragment thereof does not detach from the solid support). A solid support or substrate can be any physically separable solid to which an anti-AXL antibody or antigen-binding fragment thereof can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support or substrate can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). An anti-AXL antibody or antigen-binding fragment thereof may be linked to a solid support by covalent bonds or by non-covalent interactions and may be linked to a solid support directly or indirectly (e.g., via an intermediary agent such as a spacer molecule or biotin).

Antibodies or antigen-binding fragments thereof provided herein may be employed in any known assay method, such as flow cytometry, immunohistochemistry, immunofluorescence, mass cytometry (e.g., Cytof instrument), competitive binding assays, direct and indirect sandwich assays, and immunoprecipitation assays. Zola, Monoclonal Antibodies: A Manual of Techniques, pp. 147-158 (CRC Press, Inc. 1987).

Flow cytometry and mass cytometry assays generally involve the use of a single primary antibody to specifically identify the presence of the target molecule expressed on the surface of a dispersed suspension of individual cells. The dispersed cells are typically obtained from a biological fluid sample, e.g., blood, but may also be obtained from a dispersion of single cells prepared from a solid tissue sample such as spleen or tumor biopsy. The primary antibody may be directly conjugated with a detectable moiety, e.g., a fluorophore such as phycoerythrin for flow cytometry or a heavy metal chelate for mass cytometry. Alternatively, the primary antibody may be unlabeled or labeled with an undetectable tag such as biotin, and the primary antibody is then detected by a detectably labeled secondary antibody that specifically recognizes the primary antibody itself or the tag on the primary antibody. The labeled cells are then analyzed in an instrument capable of single cell detection, e.g., flow cytometer, mass cytometer, fluorescence microscope or brightfield light microscope, to identify those individual cells in the dispersed population or tissue sample that express the target recognized by the primary antibody. Detailed description of the technological basis and practical application of flow cytometry principles may be found in, e.g., Shapiro, Practical Flow Cytometry, 4th Edition, Wiley, 2003.

Sandwich assays involve the use of two antibodies, each capable of binding to a different immunogenic portion, or epitope, of the protein that is detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. In a cell ELISA, the target cell population may be attached to the solid support using antibodies first attached to the support and that recognize different cell surface proteins. These first antibodies capture the cells to the support. AXL on the surface of the cells can then be detected by adding anti-AXL antibody to the captured cells and detecting the amount of AXL antibody attached to the cells. In certain instances, fixed and permeabilized cells may be used, an in such instances, surface AXL and intracellular AXL may be detected.

For immunohistochemistry, the blood or tissue sample may be fresh or frozen or may be embedded in paraffin and fixed with a preservative such as formalin, for example.

The antibodies or antigen-binding fragments thereof herein also may be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintillography.

### Detection of AXL in Immune Cells

Provided herein are antibodies or antigen-binding fragments thereof and methods for detecting AXL in immune cells. Detection of AXL in immune cells may refer to detection on the surface of immune cells (e.g., by surface staining) and/or inside immune cells (e.g., by intracellular staining). In some embodiments, antibodies or antigen-binding fragments thereof and methods are provided for detecting AXL in a heterogeneous population of immune cells. A heterogeneous population of immune cells may comprise two or more types of immune cells. For example, a heterogeneous population of immune cells may comprise two or more B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and the like. In some embodiments, a heterogeneous population of immune cells comprises peripheral blood mononuclear cells (PBMCs) which may include, for example, T cells, B cells, natural killer cells, and monocytes.

Generally, cells are contacted with an anti-AXL antibody or antigen-binding fragment thereof described herein (e.g., in a flow cytometry assay as described in the Examples; or any suitable protein or cell detection assay). In some embodiments, AXL is detected at a significant level in certain immune cells by an anti-AXL antibody or antigen-binding fragment thereof described herein. AXL may be detected at a significant level by an anti-AXL antibody or antigen-binding fragment thereof described herein in certain immune cells and not significantly detected in other immune cells. The level of AXL detection in certain immune cells may vary according to certain factors such as, for example, type of detection assay, type of detection reagent (e.g., type of dye), antibody concentration, donor cell variability, and the like.

In some of any embodiments, any of the antibodies or antigen binding fragments thereof provided herein can be used in the characterization of single cells by measurement of gene-expression levels and cellular proteins. Among such known single cell sequencing platforms suitable for integration with the antibodies or antigen binding fragments thereof described herein is the Drop-seq method, including, but not limited to, microfluidic, plate-based, or microwell, Seq-Well™ method and adaptations of the basic protocol, and InDrop™ method.

In another embodiment, a single cell sequencing platform suitable for integration with the antibodies or antigen binding fragments thereof described herein is 10× genomics single cell 3′ solution or single cell V (D) J solution, either run on Chromium controller, or dedicated Chromium single cell controller. Other suitable sequencing methods include Wafergen iCel18™ method, Microwell-seq method, Fluidigm CI™ method and equivalent single cell products. Still other known sequencing protocols useful with the antibodies or antigen binding fragments thereof described herein include BD Resolve™ single cell analysis platform and ddSeq (from Illumina® Bio-Rad® SureCell™ WTA 3′ Library Prep Kit for the ddSEQ™ System, 2017, Pub. No. 1070-2016-014-B, Illumina Inc., Bio-Rad Laboratories, Inc.). In still other embodiment, the antibodies or antigen binding fragments thereof described herein are useful with combinatorial indexing-based approaches (sci-RNA-seq™ method or SPLIT-seq™ method) and Spatial Transcriptomics, or comparable spatially resolved sequencing approaches. The methods and compositions described herein can also be used as an added layer of information on standard index sorting (FACS) and mRNA-sequencing-based approaches.

In some of any embodiments, any of the antibodies or antigen binding fragments thereof described herein can be used to detect the presence, absence or amount of the various nucleic acids, proteins, targets, oligonucleotides, amplification products and barcodes described herein.

In some of any embodiments, the detection comprises hybridization of a detectable moiety to the antibody or antigen binding fragment thereof. In some of any embodiments, the sample is contacted with a second antibody. In some of any embodiments, the second antibody is an antibody comprising a detectable moiety. In some of any embodiments, the detectable moiety comprises an oligonucleotide. In some of any embodiments, the detectable moiety comprises a fluorescent label. In some of any embodiments, the measurement comprises sequencing. In some of any embodiments, the detectable moiety comprises immunofluorescence. In some of any embodiments, the sample is a formalin-fixed paraffin-embedded sample. In some of any embodiments, the sample comprises a cell. In some of any embodiments, the sample comprises a tissue sample.

Detection of AXL at a significant level may refer to a particular signal to noise (S:N) ratio (e.g., threshold or range) measured in a flow cytometry assay.

In some embodiments, AXL is detected at a significant level in immune cells (e.g., lymphocytes.

### Kits Incorporating Anti-AXL Antibodies or Antigen-Binding Fragments Thereof Provided Herein

An anti-AXL antibody or antigen-binding fragment thereof herein may be provided in a kit, for example, a packaged combination of reagents in predetermined amounts with instructions for use (e.g., instructions for performing a diagnostic assay; instructions for performing a laboratory assay). In some embodiments, the kit is a diagnostic kit configured to detect AXL in a sample (e.g., a biological sample). Where the anti-AXL antibody or antigen-binding fragment thereof is labeled with a fluorophore, the kit may include an identical isotype negative control irrelevant antibody to control for non-specific binding of the anti-AXL antibody or antigen-binding fragment thereof. Where the anti-AXL antibody or antigen-binding fragment thereof is labeled with an enzyme, the kit may include substrates and cofactors required by the enzyme (e.g., substrate precursor which provides the detectable chromophore or fluorophore). Additional additives may be included such as stabilizers, buffers (e.g., a block buffer or lysis buffer), and the like. The relative amounts of the various reagents may be varied widely to provide for concentrations in solution of the reagents that substantially optimize the sensitivity of the assay. In certain instances, reagents may be provided as dry powders (e.g., lyophilized powder), including excipients that on dissolution will provide a reagent solution having the appropriate concentration.

### Articles of Manufacture

In another aspect of the present technology, an article of manufacture containing materials useful for the treatment, or diagnosis, of the disorders described herein is provided. An article of manufacture may comprise a container and a label. Suitable containers include, for example, bottles, vials, syringes, and test tubes. Containers may be formed from a variety of materials such as glass or plastic. A container may hold a composition that is effective for treating a condition and may have a sterile access port (for example the container may be an intravenous solution bag or a vial having a stopper pierceable by a hypodermic injection needle). An active anti-AXL antibody or antigen-binding fragment thereof in the composition may be an anti-AXL antibody. A label on, or associated with, the container indicates that the composition is used for treating, or diagnosing a condition of choice. The article of manufacture may further comprise a second container comprising a pharmaceutically acceptable buffer, such as phosphate-buffered saline, Ringer's solution and dextrose solution; and may further include other materials desirable from a commercial and user standpoint, including other buffers, diluents, filters, needles, syringes, and package inserts with instructions for use.

### Definitions

An “acceptor human framework” generally refers to a framework comprising the amino acid sequence of a heavy chain variable domain (VH) framework or a light chain variable domain (VL) framework derived from a human immunoglobulin framework or a human consensus framework, as defined herein. An acceptor human framework “derived from” a human immunoglobulin framework or a human consensus framework may comprise the same amino acid sequence thereof, or it may contain amino acid sequence changes. In some embodiments, the number of framework amino acid changes are 10 or less, 9 or less, 8 or less, 7 or less, 6 or less, 5 or less, 4 or less, 3 or less, or 2 or less. In some embodiments, the VH and/or VL acceptor human framework(s) is (are) identical in sequence to the VH and/or VL human immunoglobulin framework amino acid sequence or human consensus framework amino acid sequence.

“Framework” or “FR” generally refers to variable domain residues other than hypervariable region (HVR) residues. The FR of a variable domain generally consists of four FR domains: FR1; FR2; FR3; and FR4. Accordingly, the HVR and FR sequences generally appear in the following sequence in VH (or VL): FR1-H1 (L1)-FR2-H2 (L2)-FR3-H3 (L3)-FR4.

A “human consensus framework” generally refers to a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

The term “hypervariable region” or “HVR” generally refers to each of the regions of an antibody variable domain that are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition.

The precise amino acid sequence boundaries of a given CDR or FR can be readily determined using any number of well-known schemes, including those described in Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745,” (“Contact” numbering scheme), Martin et al., Proc. Natl. Acad. Sci., 86:9268-9272 (1989) (“AbM” numbering scheme), Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 6, below, lists exemplary position boundaries of CDRH1, CDRH2, CDRH3, and CDRL1, CDRL2, and CDRL3 as identified by Kabat, Chothia, and Contact schemes, respectively. For CDRH1, residue numbering is listed using both the Kabat and Chothia numbering schemes. FRs are located between CDRs, for example, with FRH1 located between CDRH1 and CDRH2, and so forth. It is noted that because the shown Kabat numbering scheme places insertions at H35A and H35B, the end of the Chothia CDRH1 loop when numbered using the shown Kabat numbering convention varies between H32 and H34, depending on the length of the loop.

TABLE 6

| CDR | Kabat | Chothia | AbM | Contact |
|---|---|---|---|---|
| CDRH1 | H31--H35B | H26--H32 . . . | H26--H35B | H30--H35B |
| (Kabat | | 34 | | |
| Numbering¹) | | | | |
| CDRH1 | H31--H35 | H26--H32 | H26--H35 | H30--H35 |
| (Chothia | | | | |
| Numbering²) | | | | |
| CDRH2 | H50--H65 | H52--H56 | H50--H58 | H47--H58 |
| CDRH3 | H95--H102 | H95--H102 | H95--H102 | H93--H101 |
| CDRL1 | L24--L34 | L24--L34 | L24--L34 | L30--L36 |
| CDRL2 | L50--L56 | L50--L56 | L50--L56 | L46--L55 |
| CDRL3 | L89--L97 | L89--L97 | L89--L97 | L89--L96 |

¹Kabat et al. (1991), “Sequences of Proteins of Immunological Interest,” 5^{th} Ed. Public Health Service, National Institutes of Health, Bethesda, MD
²Al-Lazikani et al., (1997) JMB 273, 927-948

CDRs also comprise “specificity determining residues,” or “SDRs,” which are residues that contact a particular antigen. Unless otherwise indicated, HVR residues and other residues in the variable domain (e.g., FR residues) are numbered herein according to Kabat et al., supra.

The term “variable region” or “variable domain” generally refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al. Kuby Immunology, 6th ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Affinity” generally refers to the strength of the sum total of non-covalent interactions between a single binding site of a molecule (e.g., an antibody) and its binding partner (e.g., an antigen). Unless indicated otherwise, “binding affinity” refers to intrinsic binding affinity, which reflects a 1:1 interaction between members of a binding pair (e.g., antibody and antigen). The affinity of a molecule X for its partner Y can generally be represented by a dissociation constant (K_{d}). Affinity can be measured by common methods known in the art, including those described herein. Specific illustrative and example embodiments for measuring binding affinity are described elsewhere herein. In some instances, antibodies herein bind to a target (e.g., AXL) with a high affinity, e.g., a K_{d} value of no more than about 1×10⁻⁷ M; preferably no more than about 1×10⁻⁸ M; and preferably no more than about 5×10⁻⁹ M.

An “affinity matured” antibody generally refers to an antibody with one or more alterations in one or more hypervariable regions (HVRs), compared to a parent antibody that does not possess such alterations. Preferably, such alterations result in improved affinity of the antibody for its target antigen.

The term “anti-AXL antibody or antigen-binding fragment thereof” generally refers to a molecule that is, or comprise, one or more anti-AXL antibodies, AXL-binding antibody fragments, or AXL-binding antibody derivatives.

The terms “anti-AXL antibody” and “an antibody that binds to AXL” generally refer to an antibody capable of binding AXL with sufficient affinity and/or specificity such that the antibody is useful as a research tool, diagnostic agent and/or therapeutic agent in targeting AXL. In some embodiments, the extent of binding of an anti-AXL antibody (or antigen-binding fragment thereof) to an unrelated, non-AXL protein is less than about 10% of the binding of the antibody to AXL as measured, e.g., by a radioimmunoassay (RIA) or by Scatchard analysis or by surface plasmon resonance, such as, for example, Biacore. In certain embodiments, an antibody that binds to AXL has a dissociation constant (kD) of 0.1 μM, 100 nM, 10 nM, 1 nM, 0.1 nM, 0.01 nM, or 0.001 nM (e.g., 10⁻⁷ M or less, e.g., from 10⁻⁷ M to 10⁻¹³ M). In certain embodiments, an anti-AXL antibody binds to an epitope of AXL that is conserved among AXL from different species.

The term “antibody” herein is used in the broadest sense and includes polyclonal and monoclonal antibodies, including intact antibodies and functional (antigen-binding) antibody fragments, including fragment antigen-binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “antibody derivative” generally refers to a molecule other than an intact antibody that comprises a portion derived from an intact antibody (or antigen-binding fragment thereof) and that binds the antigen to which the intact antibody (or antigen-binding fragment thereof) binds. Examples of antibody derivatives include but are not limited to single chain variable fragments (scFv), diabodies, triabodies, and the like, aptamers comprising multiple antigen-binding antibody fragments, single chain variable fragments, diabodies, triabodies, and the like.

An “antibody fragment” or “antigen-binding antibody fragment” generally refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2 and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

The term “Fc region” generally refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

An “antibody that binds to the same epitope” as a reference antibody (e.g., an antibody that binds AXL) generally refers to an antibody that blocks binding of the reference antibody to its antigen in a competition assay by 50% or more, and conversely, the reference antibody blocks binding of the antibody to its antigen in a competition assay by 50% or more.

The term “chimeric” antibody generally refers to an antibody in which a portion of the heavy and/or light chain is derived from a particular source or species, while the remainder of the heavy and/or light chain is derived from a different source or species.

A “human antibody” generally refers to an antibody that possesses and amino acid sequence corresponding to that of an antibody produced by a human or a human cell or derived from a non-human source that utilizes human antibody repertoires or other human antibody-encoding sequences. This definition of a human antibody specifically excludes a “humanized” antibody comprising non-human antigen-binding residues.

A “humanized” antibody generally refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. In some embodiments, a humanized antibody (or antigen-binding fragment or derivative thereof), when aligned with the antibody from which the acceptor framework regions were derived, includes one or more amino acid substitutions (or deletions or insertions) at desired locations. In some such embodiments, the amino acid residue(s) substituted (or inserted or deleted) at a particular position in the human (or other) or other FR corresponds to the amino acid residue(s) at the corresponding location(s) in the parent antibody (i.e., the non-human antibody from which the CDRs or HVRs were derived). A “humanized form” of an antibody, e.g., a non-human antibody, refers to an antibody that has undergone humanization.

The term “antibody drug conjugate” (ADC) or “immunoconjugate” generally refers to a particular class of antibody-drug conjugates. Here, “antibody-drug conjugate” is an anti-AXL antibody or antigen-binding fragment thereof (e.g., an anti-AXL antibody or AXL-binding fragment or derivative thereof) conjugated to one or more heterologous molecule(s), including, but not limited to, a cytotoxic agent.

The term “cytotoxic agent” generally refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “diagnostic reagent” generally refers to a compound, e.g., a target-specific antibody (or antigen-binding thereof) used to perform a diagnostic assay.

“Effector functions” generally refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an antibody or antigen-binding fragment thereof, e.g., a pharmaceutical formulation, generally refers to an amount effective, at dosages and for periods of time necessary, to achieve the desired therapeutic or prophylactic result.

The term “epitope” generally refers to the particular site on an antigen molecule to which an antibody binds.

The terms “host cell”, “host cell line”, and “host cell culture” are used interchangeably and generally refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “rabbit antibody” generally refers to an antibody that possesses an amino acid sequence that corresponds to that of an antibody produced by a rabbit or a rabbit cell or derived from a non-rabbit source that utilizes rabbit antibody repertoires or other rabbit antibody-encoding sequences.

An “immunoconjugate” generally refers to an antibody (or antigen-binding fragment or derivative thereof) conjugated to one or more heterologous molecule(s), including, but not limited to, a cytotoxic agent. An immunoconjugate is equivalent to the term “antibody drug conjugate” (ADC).

An “individual” or “patient” or “subject” generally is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” molecule (e.g., nucleic acid, antibody) generally refers to a molecule that has been separated from a component of its original environment (e.g., the natural environment if it is naturally occurring, or a host cell if expressed exogenously), and thus is altered by human intervention (e.g., “by the hand of man”) from its original environment. In some embodiments, for example, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). An isolated nucleic acid may refer to a nucleic acid molecule contained in cells that ordinarily contain the nucleic acid molecule, but the nucleic acid molecule is present extrachromosomally or at a chromosomal location that is different from its natural chromosomal location. In some embodiments, an isolated nucleic acid can be provided with fewer non-nucleic acid components (e.g., protein, lipid) than the amount of components that are present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components.

“Isolated nucleic acid encoding an anti-AXL antibody” or “isolated polynucleotide encoding an anti-AXL antibody” generally refers to one or more nucleic acid molecules encoding antibody heavy and light chains (or fragments thereof), including such nucleic acid molecule(s) in a single vector or separate vectors, and such nucleic acid molecule(s) present at one or more locations in a recombinant host cell.

The term “AXL” generally refers to any native, mature AXL that results from processing of a AXL precursor protein in a cell. The term includes AXL from any vertebrate source, including mammals such as primates (e.g., humans and cynomolgus or rhesus monkeys) and rodents (e.g., mice and rats), unless otherwise indicated. The term also includes naturally occurring variants of AXL, e.g., splice variants or allelic variants. The amino acid sequence of an example of human AXL protein is shown in SEQ ID NO:37.

The term “AXL-positive cell” generally refers to any cell that expresses AXL on its surface or on an intracellular membrane or organelle (e.g., endosome, ER, Golgi apparatus, lysosome, and the like). Some cells, such as immune cells (e.g., lymphocytes), exhibit up-regulation of AXL expression.

The term “monoclonal antibody” generally refers to an antibody obtained from a population of substantially homogeneous antibodies, i.e., the individual antibodies comprising the population are identical (as assessed at the level of Ig heavy and/or light chain amino acid sequence) and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present technology may be made by a variety of techniques, including, but not limited to, the hybridoma method, recombinant DNA methods, phage-display methods, and methods utilizing transgenic animals containing all or part of the human immunoglobulin loci, such methods and other example methods for making monoclonal antibodies being described herein.

The term “package insert” generally refers to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence generally refers to the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “pharmaceutical composition” generally refers to a preparation that is in such form as to permit the biological activity of an active ingredient contained therein to be effective, and which contains no additional components which are unacceptably toxic to a subject to which the formulation would be administered.

A “pharmaceutically acceptable carrier” generally refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) generally refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies herein are used to delay development of a disease or to slow the progression of a disease.

The term “vector” generally refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### EXEMPLARY EMBODIMENTS

Exemplary embodiments provided in accordance with the presently disclosed subject matter include, but are not limited to, the following embodiments:
1. An isolated antibody or antigen binding fragment thereof that specifically binds to AXL, wherein the isolated antibody comprises:
a) a heavy chain variable region comprising:
(i) a heavy chain complementarity determining region 1 (CDRH1) comprising the sequence of X₁X₂X₃X₄X₅X₆ (SEQ ID NO:57), wherein X₁ is S, N or D, X₂ is A or Y, X₃ is Y or G, X₄ is S or M, X₅ is W, N or Y, and X₆ is H or no amino acid;
(ii) a heavy chain complementarity determining region 2 (CDRH2) comprising the sequence of X₁IX₃X₄X₅X₆X₇X₈X₉X₁₀X₁₁X₁₂X₁₃X₁₄X₁₅X₁₆X₁₇ (SEQ ID NO:58), wherein X₁ is Y or W, X₃ is H, N or S, X₄ is Y, T or N, X₅ is S, Y or G, X₆ is G, I or T, X₇ is S or G, X₈ is T, E or S, X₉ is N, P or T, X₁₀ is Y or T, X₁₁ is N or Y, X₁₂ is Por A, X₁₃ is S or D, X₁₄ is L, D or T, X₁₅ is K, F or V, X₁₆ is S or K, and X₁₇ is G or no amino acid;
(iii) a heavy chain complementarity determining region 3 (CDRH3) comprising the sequence of X₁X₂X₃FYAMDY (SEQ ID NO:59), wherein X₁ is S, G or no amino acid, X₂ is L, T or D, and X₃ is W, T, R or H.; and
b) a light chain variable region comprising:
(iv) a light chain complementarity determining region 1 (CDRL1) comprising the sequence of X₁ASX₄X₅X₆X₇X₈X₉X₁₀X₁₁X₁₂ (SEQ ID NO:60), wherein X₁ is R, H, S or K, X₄ is K, Q or S, X₅ is S, N or D, X₆ is I or V, X₇ is S or N, X₈ is K, V, S or T, X₉ is Y, V, S or T, X₁₀ is L, Y or V, X₁₁ is A, N or L, and X₁₂ is H or no amino acid;
(v) a light chain complementarity determining region 2 (CDRL2) comprising the sequence of X₁X₂SX₄X₅X₆X₇ (SEQ ID NO:61), wherein X₁ is S, K, R or W, X₂ is G, A or T, X₄ is T or N, X₅ is L or R, X₆ is Q, H or A, X₇ is S or T; and
(vi) a light chain complementarity determining region 3 (CDRL3) comprising the sequence of QQX₃X₄X₅X₆X₇X₈X₉ (SEQ ID NO:62), wherein X₃ is H or G, X₄ is N, Q, S or Y, X₅ is E, S or N, X₆ Y, I or T, X₇ is P, L or F, X₈ is W, L, T or F, X₉ is T or no amino acid.
2. The isolated antibody or antigen binding fragment thereof of embodiment 1, wherein:
(1) the CDRH1 comprises the sequence set forth in any of SEQ ID NOS: 24, 25, and 26 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 24, 25 and 26;
(2) the CDRH2 comprises the sequence set forth in any of SEQ ID NOS: 27, 28, 29 and 30 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 27, 28, 29 and 30; and
(3) the CDRH3 comprises the sequence set forth in any of SEQ ID NOS: 31, 32, 33 and 34 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 31, 32, 33 and 34.
3. The isolated antibody or antigen binding fragment thereof embodiment 1 or 2, wherein:
(1) the CDRH1 comprises the sequence set forth in any of SEQ ID NOS: 24, 25 and 26 or a sequence having one amino acid substitution relative to a sequence of any one of SEQ ID NOS: 24, 25 and 26;
(2) the CDRH2 comprises the sequence set forth in any of SEQ ID NOS: 27, 28, 29 and 30 or a sequence having one, two, or three amino acid substitutions relative to a sequence of any one of SEQ ID NOS: 27, 28, 29 and 30; and
(3) the CDRH3 comprises the sequence set forth in any of SEQ ID NOS: 31, 32, 33 and 34 or a sequence having one or two amino acid substitutions relative to a sequence of any one of SEQ ID NOS: 31, 32, 33 and 34.
4. The isolated antibody or antigen binding fragment thereof of any one of embodiments 1 to 3, wherein:
(1) the CDRH1 comprises the sequence set forth in any of SEQ ID NOS: 24, 25 and 26;
(2) the CDRH2 comprises the sequence set forth in any of SEQ ID NOS: 27, 28, 29 and 30; and
(3) the CDRH3 comprises the sequence set forth in any of SEQ ID NOS: 31, 32, 33 and 34.
5. The isolated antibody or antigen binding fragment thereof of any one of embodiments 1 to 4, wherein the CDRH1 comprises the sequence set forth in SEQ ID NO:24, the CDRH2 comprises the sequence set forth in SEQ ID NO:27, and the CDRH3 comprises the sequence set forth in SEQ ID NO:31.
6 The isolated antibody or antigen binding fragment thereof of any one of embodiments 1 to 4, wherein the CDRH1 comprises the sequence set forth in SEQ ID NO:25, the CDRH2 comprises the sequence set forth in SEQ ID NO:28, and the CDRH3 comprises the sequence set forth in SEQ ID NO:32.
7. The isolated antibody or antigen binding fragment thereof of any one of embodiments 1 to 4, wherein the CDRH1 comprises the sequence set forth in SEQ ID NO:25, the CDRH2 comprises the sequence set forth in SEQ ID NO:29, and the CDRH3 comprises the sequence set forth in SEQ ID NO:33.
8 The isolated antibody or antigen binding fragment thereof of any one of embodiments 1 to 4, wherein the CDRH1 comprises the sequence set forth in SEQ ID NO:26, the CDRH2 comprises the sequence set forth in SEQ ID NO:30, and the CDRH3 comprises the sequence set forth in SEQ ID NO:34.
9. The isolated antibody or antigen binding fragment thereof of any one of embodiments 1 to 8, wherein:
(1) the CDRL1 comprises the sequence set forth in any one of SEQ ID NOS: 35, 36, 37 and 38 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 35, 36, 37 and 38;
(2) the CDRL2 comprises the sequence set forth in any one of SEQ ID NOS: 39, 40, 41, 42 and 43 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 39, 40, 41, 42 and 43; and
(3) the CDRL3 comprises the sequence set forth in any one of SEQ ID NOS: 44, 45, 46, 47 and 48 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 44, 45, 46, 47 and 48.
10. The isolated antibody or antigen binding fragment thereof of embodiment 1 or 9, wherein:
(1) the CDRL1 comprises the sequence set forth in any one of SEQ ID NOS: 35, 36, 37 and 38 or a sequence having one or two amino acid substitutions relative to a sequence of any one of SEQ ID NOS: 35, 36, 37 and 38;
(2) the CDRL2 comprises the sequence set forth in any one of SEQ ID NOS: 39, 40, 41, 42 and 43 or a sequence having one amino acid substitution relative to a sequence of any one of SEQ ID NOS: 39, 40, 41, 42 and 43; and
(3) the CDRL3 comprises the sequence set forth in any one of SEQ ID NOS: 44, 45, 46, 47 and 48 or a sequence having one or two amino acid substitutions relative to a sequence of any one of SEQ ID NOS: 44, 45, 46, 47 and 48.
10. The isolated antibody or antigen binding fragment thereof of any one of embodiments 1 to 9, wherein:
(1) the CDRL1 comprises the sequence set forth in any one of SEQ ID NOS: 35, 36, 37 and 38;
(2) the CDRL2 comprises the sequence set forth in any one of SEQ ID NOS: 39, 40, 41, 42 and 43; and
(3) the CDRL3 comprises the sequence set forth in any one of SEQ ID NOS: 44, 45, 46, 47 and 48.
11. The isolated antibody or antigen binding fragment thereof of any one of embodiments 1 to 10, wherein the CDRL1 comprises the sequence set forth in SEQ ID NO: 35, the CDRL2 comprises the sequence set forth in SEQ ID NO:39, and the CDRL3 comprises the sequence set forth in SEQ ID NO:44.
12. The isolated antibody or antigen binding fragment thereof of any one of embodiments 1 to 10, wherein the CDRL1 comprises the sequence set forth in SEQ ID NO: 36, a CDRL2 comprises the sequence set forth in SEQ ID NO:40, and a CDRL3 comprises the sequence set forth in SEQ ID NO:45.
13. The isolated antibody or antigen binding fragment thereof of any one of embodiments 1 to 10, wherein the CDRL1 comprises the sequence set forth in SEQ ID NO: 37, a CDRL2 comprises the sequence set forth in SEQ ID NO:41, and a CDRL3 comprises the sequence set forth in SEQ ID NO:46.
14. The isolated antibody or antigen binding fragment thereof of any one of embodiments 1 to 10, wherein the CDRL1 comprises the sequence set forth in SEQ ID NO: 35, the CDRL2 comprises the sequence set forth in SEQ ID NO:42, and the CDRL3 comprises the sequence set forth in SEQ ID NO:44.
15. The isolated antibody or antigen binding fragment thereof of any one of embodiments 1 to 10, wherein the CDRL1 comprises the sequence set forth in SEQ ID NO: 37, a CDRL2 comprises the sequence set forth in SEQ ID NO:41, and a CDRL3 comprises the sequence set forth in SEQ ID NO:47.
16. The isolated antibody or antigen binding fragment thereof of any one of embodiments 1 to 10, wherein the CDRL1 comprises the sequence set forth in SEQ ID NO: 38, the CDRL2 comprises the sequence set forth in SEQ ID NO:43, and the CDRL3 comprises the sequence set forth in SEQ ID NO:48.
17. The isolated antibody or antigen binding fragment thereof of any one of embodiments 1 to 16, wherein:
(1) the CDRH1 comprises the sequence set forth in any one of SEQ ID NOS: 24, 25 and 26, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 24, 25 and 26;
(2) the CDRH2 comprises the sequence set forth in any one of SEQ ID NOS: 27, 28, 29 and 30, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 27, 28, 29 and 30;
(3) the CDRH3 comprises the sequence set forth in any one of SEQ ID NOS: 31, 32, 33 and 34, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 31, 32, 33 and 34;
(4) the CDRL1 comprises the sequence set forth in any one of SEQ ID NOS: 35, 36, 37 and 38, or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 35, 36, 37 and 38;
(5) the CDRL2 comprises the sequence set forth in any one of SEQ ID NOS: 39, 40, 41, 42 and 43 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 39, 40, 41, 42 and 43; and
(6) the CDRL3 comprises the sequence set forth in any one of SEQ ID NOS: 44, 45, 46, 47 and 48 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NOS: 44, 45, 46, 47 and 48.
18. The isolated antibody or antigen binding fragment thereof of embodiment 1 to 17, wherein:
(1) the CDRH1 comprises the sequence set forth in any one of SEQ ID NOS: 24, 25 and 26 or a sequence having one amino acid substitution relative to a sequence of any one of SEQ ID NOS: 24, 25 and 26;
(2) the CDRH2 comprises the sequence set forth in any one of SEQ ID NOS: 27, 28, 29 and 30 or a sequence having one, two, or three amino acid substitutions relative to a sequence of any one of SEQ ID NOS: 27, 28, 29 and 30;
(3) the CDRH3 comprises the sequence set forth in any one of SEQ ID NOS: 31, 32, 33 and 34 or a sequence having one or two amino acid substitutions relative to a sequence of any one of SEQ ID NOS: 31, 32, 33 and 34;
(4) the CDRL1 comprises the sequence set forth in any one of SEQ ID NOS: 35, 36, 37 and 38 or a sequence having one or two amino acid substitutions relative to a sequence of any one of SEQ ID NOS: 35, 36, 37 and 38;
(5) the CDRL2 comprises the sequence set forth in any one of SEQ ID NOS: 39, 40, 41, 42 and 43 or a sequence having one amino acid substitution relative to a sequence of any one of SEQ ID NOS: 39, 40, 41, 42 and 43; and
(6) the CDRL3 comprises the sequence set forth in any one of SEQ ID NOS: 44, 45, 46, 47 and 48 or a sequence having one or two amino acid substitutions relative to a sequence of any one of SEQ ID NOS: 44, 45, 46, 47 and 48.
19. The isolated antibody or antigen binding fragment thereof of any one of embodiments 1 to 18, wherein:
(1) the CDRH1 comprises the sequence set forth in any one of SEQ ID NOS: 24, 25 and 26;
(2) the CDRH2 comprises the sequence set forth in any one of SEQ ID NOS: 27, 28, 29 and 30;
(3) the CDRH3 comprises the sequence set forth in any one of SEQ ID NOS: 31, 32, 33 and 34:
(4) the CDRL1 comprises the sequence set forth in any one of SEQ ID NOS: 35, 36, 37 and 38;
(5) the CDRL2 comprises the sequence set forth in any one of SEQ ID NOS: 39, 40, 41, 42 and 43; and
(6) the CDRL3 comprises the sequence set forth in any one of SEQ ID NOS: 44, 45, 46, 47 and 48.
20. The isolated antibody or antigen binding fragment thereof of any one of embodiments 1 to 19, wherein the CDRH1 comprises the sequence set forth in SEQ ID NO: 24, the CDRH2 comprises the sequence set forth in SEQ ID NO:27, and the CDRH3 comprises the sequence set forth in SEQ ID NO:31, the CDRL1 comprises the sequence set forth in SEQ ID NO:35, the CDRL2 comprises the sequence set forth in SEQ ID NO:39, and the CDRL3 comprises the sequence set forth in SEQ ID NO:44.
21. The isolated antibody or antigen binding fragment thereof of any one of embodiments 1 to 19, wherein the CDRH1 comprises the sequence set forth in SEQ ID NO: 24, the CDRH2 comprises the sequence set forth in SEQ ID NO:27, and the CDRH3 comprises the sequence set forth in SEQ ID NO:31, the CDRL1 comprises the sequence set forth in SEQ ID NO:36, the CDRL2 comprises the sequence set forth in SEQ ID NO:40, and the CDRL3 comprises the sequence set forth in SEQ ID NO:45.
22. The isolated antibody or antigen binding fragment thereof of any one of embodiments 1 to 19, wherein the CDRH1 comprises the sequence set forth in SEQ ID NO: 25, the CDRH2 comprises the sequence set forth in SEQ ID NO:28, and the CDRH3 comprises the sequence set forth in SEQ ID NO:32, the CDRL1 comprises the sequence set forth in SEQ ID NO:37, the CDRL2 comprises the sequence set forth in SEQ ID NO:41, and the CDRL3 comprises the sequence set forth in SEQ ID NO:46.
23 The isolated antibody or antigen binding fragment thereof of any one of embodiments 1 to 19, wherein the CDRH1 comprises the sequence set forth in SEQ ID NO: 24, the CDRH2 comprises the sequence set forth in SEQ ID NO:27, and the CDRH3 comprises the sequence set forth in SEQ ID NO:31, the CDRL1 comprises the sequence set forth in SEQ ID NO:35, the CDRL2 comprises the sequence set forth in SEQ ID NO:42, and the CDRL3 comprises the sequence set forth in SEQ ID NO:44.
24. The isolated antibody or antigen binding fragment thereof of any one of embodiments 1 to 19, wherein the CDRH1 comprises the sequence set forth in SEQ ID NO: 25, the CDRH2 comprises the sequence set forth in SEQ ID NO:29, and the CDRH3 comprises the sequence set forth in SEQ ID NO:33, the CDRL1 comprises the sequence set forth in SEQ ID NO:37, the CDRL2 comprises the sequence set forth in SEQ ID NO:41, and the CDRL3 comprises the sequence set forth in SEQ ID NO:47.
25 The isolated antibody or antigen binding fragment thereof of any one of embodiments 1 to 19, wherein the CDRH1 comprises the sequence set forth in SEQ ID NO: 26, the CDRH2 comprises the sequence set forth in SEQ ID NO:30, and the CDRH3 comprises the sequence set forth in SEQ ID NO:34, the CDRL1 comprises the sequence set forth in SEQ ID NO:38, the CDRL2 comprises the sequence set forth in SEQ ID NO:43, and the CDRL3 comprises the sequence set forth in SEQ ID NO:48.
26. The isolated antibody or antigen binding fragment thereof of any one of embodiments 1 to 25, wherein the heavy chain variable region comprises at least 90% identity to the sequence set forth in any one of SEQ ID NOS: 1-5.
27. The isolated antibody or antigen binding fragment thereof of any of embodiments 1 to 26, wherein the heavy chain variable region comprises at least 90% identity to the sequence set forth in SEQ ID NO:1.
28. The isolated antibody or antigen binding fragment thereof of any of embodiments 1 to 26, wherein the heavy chain variable region comprises at least 90% identity to the sequence of SEQ ID NO:2.
29. The isolated antibody or antigen binding fragment thereof of any of embodiments 1 to 26, wherein the heavy chain variable region comprises at least 90% identity to the sequence set forth in SEQ ID NO:3.
30. The isolated antibody or antigen binding fragment thereof of any of embodiments 1 to 26, wherein the heavy chain variable region comprises at least 90% identity to the sequence of SEQ ID NO:4.
31. The isolated antibody or antigen binding fragment thereof of any of embodiments 1 to 26, wherein the heavy chain variable region comprises at least 90% identity to the sequence of SEQ ID NO:5.
32. The isolated antibody or antigen binding fragment thereof of any one of embodiments 1 to 26, wherein the heavy chain variable region comprises the sequence of any one of SEQ ID NOs: 1-5.
33. The isolated antibody or antigen binding fragment thereof of embodiment 32, wherein the heavy chain variable region comprises the sequence set forth in SEQ ID NO:1.
34. The isolated antibody or antigen binding fragment thereof of embodiment 32, wherein the heavy chain variable region comprises the sequence of SEQ ID NO:2.
35. The isolated antibody or antigen binding fragment thereof of embodiment 32, wherein the heavy chain variable region comprises the sequence set forth in SEQ ID NO:3.
36. The isolated antibody or antigen binding fragment thereof of embodiment 32, wherein the heavy chain variable region comprises the sequence of SEQ ID NO:4.
37. The isolated antibody or antigen binding fragment thereof of embodiment 32, wherein the heavy chain variable region comprises the sequence of SEQ ID NO:5.
38. The isolated antibody or antigen binding fragment thereof of any one of embodiments 1 to 26, wherein the light chain variable region comprises at least 90% identity to a sequence set forth in any of SEQ ID NOS: 6-11.
39. The isolated antibody or antigen binding fragment thereof of embodiment 38, wherein the light chain variable region comprises at least 90% identity to the sequence set forth in SEQ ID NO:6.
40. The isolated antibody or antigen binding fragment thereof of embodiment 38, wherein the light chain variable region comprises at least 90% identity to the sequence set forth in SEQ ID NO:7.
41. The isolated antibody or antigen binding fragment thereof of embodiment 38, wherein the light chain variable region comprises at least 90% identity to the sequence set forth in SEQ ID NO:8.
42. The isolated antibody or antigen binding fragment thereof of embodiment 38, wherein the light chain variable region comprises at least 90% identity to the sequence set forth in SEQ ID NO:9.
43. The isolated antibody or antigen binding fragment thereof of embodiment 38, wherein the light chain variable region comprises at least 90% identity to the sequence set forth in SEQ ID NO: 10.
44. The isolated antibody or antigen binding fragment thereof of embodiment 38, wherein the light chain variable region comprises at least 90% identity to the sequence set forth in SEQ ID NO:11.
45. The isolated antibody or antigen binding fragment thereof of any one of embodiments 1 to 38, wherein the light chain variable region comprises the sequence set forth in any of SEQ ID NOS: 6-11.
46. The isolated antibody or antigen binding fragment thereof of embodiment 45, wherein the light chain variable region comprises the sequence set forth in SEQ ID NO:6.
47. The isolated antibody or antigen binding fragment thereof of embodiment 45, wherein the light chain variable region comprises the sequence set forth in SEQ ID NO:7.
48. The isolated antibody or antigen binding fragment thereof of embodiment 45, wherein the light chain variable region comprises the sequence set forth in SEQ ID NO:8.
49. The isolated antibody or antigen binding fragment thereof of embodiment 45, wherein the light chain variable region comprises the sequence set forth in SEQ ID NO:9.
50. The isolated antibody or antigen binding fragment thereof of embodiment 45, wherein the light chain variable region comprises the sequence set forth in SEQ ID NO:10.
51. The isolated antibody or antigen binding fragment thereof of embodiment 45, wherein the light chain variable region comprises the sequence set forth in SEQ ID NO:11.
52. The isolated antibody or antigen binding fragment thereof of any one of embodiments 1 to 51, wherein the heavy chain variable region comprises at least 90% identity to the sequence set forth in any one of SEQ ID NOS: 1-5, and wherein the light chain variable region comprises at least 90% identity to the sequence set forth in any of SEQ ID NOS: 6-11.
53. The isolated antibody or antigen binding fragment thereof of any one of embodiments 1 to 52, wherein the heavy chain variable region comprises at least 90% identity to the sequence set forth in SEQ ID NO: 1, and wherein the light chain variable region comprises at least 90% identity to the sequence set forth in SEQ ID NO:6.
54 The isolated antibody or antigen binding fragment thereof of any one of embodiments 1 to 52, wherein the heavy chain variable region comprises at least 90% identity to the sequence set forth in SEQ ID NO:1, and wherein the light chain variable region comprises at least 90% identity to the sequence set forth in SEQ ID NO:7.
55. The isolated antibody or antigen binding fragment thereof of any one of embodiments 1 to 52, wherein the heavy chain variable region comprises at least 90% identity to the sequence set forth in SEQ ID NO:2, and wherein the light chain variable region comprises at least 90% identity to the sequence set forth in SEQ ID NO:8.
56. The isolated antibody or antigen binding fragment thereof of any one of embodiments 1 to 52, wherein the heavy chain variable region comprises at least 90% identity to the sequence set forth in SEQ ID NO:3, and wherein the light chain variable region comprises at least 90% identity to the sequence set forth in SEQ ID NO:9.
57. The isolated antibody or antigen binding fragment thereof of any one of embodiments 1 to 52, wherein the heavy chain variable region comprises at least 90% identity to the sequence set forth in SEQ ID NO:4, and wherein the light chain variable region comprises at least 90% identity to the sequence set forth in SEQ ID NO:10.
58. The isolated antibody or antigen binding fragment thereof of any one of embodiments 1 to 52, wherein the heavy chain variable region comprises at least 90% identity to the sequence set forth in SEQ ID NO:5, and wherein the light chain variable region comprises at least 90% identity to the sequence set forth in SEQ ID NO:11.
59. The isolated antibody or antigen binding fragment thereof of any one of embodiments 1 to 58, wherein the heavy chain variable region comprises the sequence set forth in any one of SEQ ID NOS: 1-5, and wherein the light chain variable region comprises the sequence set forth in any of SEQ ID NOS: 6-11.
60. The isolated antibody or antigen binding fragment thereof of any one of embodiments 1 to 58, wherein the heavy chain variable region comprises the sequence set forth in SEQ ID NO:1, and wherein the light chain variable region comprises the sequence set forth in SEQ ID NO:6.
61. The isolated antibody or antigen binding fragment thereof of any one of embodiments 1 to 58, wherein the heavy chain variable region comprises the sequence set forth in SEQ ID NO:1, and wherein the light chain variable region the sequence set forth in SEQ ID NO:7.
62. The isolated antibody or antigen binding fragment thereof of any one of embodiments 1 to 58, wherein the heavy chain variable region comprises the sequence set forth in SEQ ID NO:2, and wherein the light chain variable region comprises the sequence set forth in SEQ ID NO:8.
63. The isolated antibody or antigen binding fragment thereof of any one of embodiments 1 to 58, wherein the heavy chain variable region comprises the sequence set forth in SEQ ID NO:3, and wherein the light chain variable region comprises the sequence set forth in SEQ ID NO:9.
64. The isolated antibody or antigen binding fragment thereof of any one of embodiments 1 to 58, wherein the heavy chain variable region comprises the sequence set forth in SEQ ID NO:4, and wherein the light chain variable region the sequence set forth in SEQ ID NO:10.
65. The isolated antibody or antigen binding fragment thereof of any one of embodiments 1 to 58, wherein the heavy chain variable region comprises the sequence set forth in SEQ ID NO:5, and wherein the light chain variable region comprises the sequence set forth in SEQ ID NO:11.
66. The isolated antibody or antigen binding fragment thereof of any one of embodiments 1 to 65, wherein the heavy chain variable region further comprises a signal sequence.
67. The isolated antibody or antigen binding fragment thereof of embodiment 66, wherein the signal sequence comprises the sequence set forth in any of SEQ ID NOS: 49-51.
68. The isolated antibody or antigen binding fragment thereof of any one of embodiments 1 to 67, wherein the light chain variable region further comprises a signal sequence.
69. The isolated antibody or antigen binding fragment thereof of embodiments 68, wherein the signal sequence comprises the sequence set forth in any of SEQ ID NOS: 52-55.
70 The isolated antibody or antigen binding fragment thereof of any one of embodiments 1 to 69, wherein the antibody comprises an Fc polypeptide having at least 90% identity to a sequence of SEQ ID NO:63.
71. An isolated antibody or antigen binding fragment thereof that specifically binds to AXL wherein the isolated antibody competes binding to the AXL receptor with an antibody of any one of embodiments 1 to 70.
72. The isolated antibody or antigen binding fragment thereof of any one of embodiments 1 to 71, wherein the antibody is a monoclonal antibody.
73. The isolated antibody or antigen binding fragment thereof of any one of embodiments 1 to 72, wherein the antibody is a humanized antibody.
74. The isolated antibody or antigen binding fragment thereof of any one of embodiments 1 to 73, wherein the antibody comprises one or more human framework regions.
75. The isolated antibody or antigen binding fragment thereof of any one of embodiments 1 to 74, wherein the antibody is conjugated to a detectable marker or label.
76 The isolated antibody or antigen binding fragment thereof of any one of embodiments 1 to 75, wherein the antibody is non-diffusively immobilized on a solid support.
77. An isolated nucleic acid encoding the isolated antibody or antigen binding fragment thereof of any one of embodiments 1 to 76.
78. An isolated nucleic acid comprising a nucleotide sequence that encodes a heavy chain variable region comprising at least 90% sequence identity to the sequence set forth in any of SEQ ID NOS: 12-17.
79. The isolated nucleic acid of embodiment 78, wherein the heavy chain variable region comprises at least 90% sequence identity to the sequence set forth in SEQ ID NO: 12.
80. The isolated nucleic acid of embodiment 78, wherein the heavy chain variable region comprises at least 90% sequence identity to the sequence set forth in SEQ ID NO:13.
81. The isolated nucleic acid of embodiment 78, wherein the heavy chain variable region comprises at least 90% sequence identity to the sequence set forth in SEQ ID NO: 14.
82. The isolated nucleic acid of embodiment 78, wherein the heavy chain variable region comprises at least 90% sequence identity to the sequence set forth in SEQ ID NO: 15.
83 The isolated nucleic acid of embodiment 78, wherein the heavy chain variable region comprises at least 90% sequence identity to the sequence set forth in SEQ ID NO:16.
84. The isolated nucleic acid of embodiment 78, wherein the heavy chain variable region comprises at least 90% sequence identity to the sequence set forth in SEQ ID NO: 17.
85. An isolated nucleic acid comprising a nucleotide sequence that encodes a heavy chain variable region comprising the sequence set forth in any of SEQ ID NOS: 12-17.
86. The isolated nucleic acid of embodiment 85, wherein the heavy chain variable region comprises the sequence set forth in SEQ ID NO:12.
87. The isolated nucleic acid of embodiment 85, wherein the heavy chain variable region comprises the sequence set forth in SEQ ID NO:13.
88. The isolated nucleic acid of embodiment 85, wherein the heavy chain variable region comprises the sequence set forth in SEQ ID NO:14.
89. The isolated nucleic acid of embodiment 85, wherein the heavy chain variable region comprises the sequence set forth in SEQ ID NO:15.
90. The isolated nucleic acid of embodiment 85, wherein the heavy chain variable region comprises the sequence set forth in SEQ ID NO: 16.
91. The isolated nucleic acid of embodiment 85, wherein the heavy chain variable region comprises the sequence set forth in SEQ ID NO:17.
92. An isolated nucleic acid comprising a nucleotide sequence that encodes a light chain variable region comprising at least 90% sequence identity to the sequence set forth in any of SEQ ID NOS: 18-23.
93. The isolated nucleic acid of embodiment 92, wherein the light chain variable region comprises at least 90% sequence identity to the sequence set forth in SEQ ID NO:18.
94. The isolated nucleic acid of embodiment 92, wherein the light chain variable region comprises at least 90% sequence identity to the sequence set forth in SEQ ID NO: 19.
95. The isolated nucleic acid of embodiment 92, wherein the light chain variable region comprises at least 90% sequence identity to the sequence set forth in SEQ ID NO:20.
96. The isolated nucleic acid of embodiment 92, wherein the light chain variable region comprises at least 90% sequence identity to the sequence set forth in SEQ ID NO:21.
97. The isolated nucleic acid of embodiment 92, wherein the light chain variable region comprises at least 90% sequence identity to the sequence set forth in SEQ ID NO:22.
98 The isolated nucleic acid of embodiment 92, wherein the light chain variable region comprises at least 90% sequence identity to the sequence set forth in SEQ ID NO:23.
99. An isolated nucleic acid comprising a nucleotide sequence that encodes a light chain variable region comprising the sequence set forth in any of SEQ ID NOS: 18-23.
100. The isolated nucleic acid of embodiment 99, wherein the light chain variable region comprises the sequence set forth in SEQ ID NO: 18.
101. The isolated nucleic acid of embodiment 99, wherein the light chain variable region comprises the sequence set forth in SEQ ID NO: 19.
102. The isolated nucleic acid of embodiment 99, wherein the light chain variable region comprises the sequence set forth in SEQ ID NO:20.
103. The isolated nucleic acid of embodiment 99, wherein the light chain variable region comprises the sequence set forth in SEQ ID NO:21.
104. The isolated nucleic acid of embodiment 99, wherein the light chain variable region comprises the sequence set forth in SEQ ID NO:22.
105. The isolated nucleic acid of embodiment 99, wherein the light chain variable region comprises the sequence set forth in SEQ ID NO:23.
106. An expression vector comprising the nucleic acid of any one of embodiments 77 to 105.
107. An isolated host cell comprising the expression vector of embodiment 106.
108. A pharmaceutical composition comprising the isolated antibody or antigen binding fragment thereof of any one of embodiments 1 to 76 and a pharmaceutically acceptable carrier.
109. A diagnostic reagent comprising the isolated antibody or antigen binding fragment thereof of any one of embodiments 1 to 76.
110. A kit comprising the isolated antibody or antigen binding fragment thereof of any one of embodiments 1 to 76 or the diagnostic reagent of embodiment 75.
111. A method of detecting AXL comprising contacting a sample known or suspected to contain AXL with the isolated antibody or antigen binding fragment thereof of any one of embodiments 1 to 76.
112. A method of detecting AXL, comprising
a) contacting a sample with the isolated antibody or antigen binding fragment thereof of any one of embodiments 1 to 76, under conditions to bind said antibody to a AXL on said sample, wherein the binding generates the production of a receptor/antibody complex; and
b) detecting the presence of the receptor/antibody complexes, wherein the detecting comprises the presence or absence of the AXL on said sample.
113. A method of treating or preventing a disease or disorder associated with AXL in a subject, comprising:
a) contacting a sample known or suspected to contain AXL with the isolated antibody or antigen binding fragment thereof of any one of embodiments 1 to 76;
b) detecting the presence of complexes comprising AXL and the antibody; wherein the presence of the complexes indicates the presence of a disease or disorder; and
c) administering to the subject the isolated antibody or antigen binding fragment thereof of any one of embodiments 1 to 76.
114. A method of diagnosing a disease or disorder, comprising:
a) isolating a sample from a subject;
b) incubating the sample with the isolated antibody or antigen binding fragment thereof of any one of embodiments 1 to 76, for a period of time sufficient to generate AXL:anti-AXL complexes;
c) detecting the presence or absence of the AXL:anti-AXL complexes from the isolated tissue; and
d) associating presence or abundance of AXL with a location of interest of a tissue sample.
115. The method of embodiment 114, wherein the increase of AXL over a control level in the location of interest of the tissue sample is indicative of a disease or disorder in a subject.
116. The method of any one of embodiments 111-115, wherein the method is performed in vitro.
117. The method of any one of embodiments 111-115, wherein the method is performed in vivo.
118. The method of any one of embodiments 111-117, wherein the detection comprises intracellular detection.
119. The method of any one of embodiments 111-118, wherein the detection comprises detection on the surface of a cell.
120. The method of any one of embodiments 111-119, wherein the detection comprises hybridization of a detectable moiety to the antibody or antigen binding fragment thereof.
121. The method of any one of embodiments 111-120, wherein the sample is contacted with a second antibody.
122. The method of any one of embodiments 111-121, wherein the second antibody is an antibody comprising a detectable moiety.
123. The method of any one of embodiments 111-122, wherein the detectable moiety comprises an oligonucleotide.
124. The method of any one of embodiments 111-123, wherein the detectable moiety comprises a fluorescent label.
125. The method of any one of embodiments 111-124, wherein the measurement comprises sequencing.
126. The method of any one of embodiments 111-125, wherein the detectable moiety comprises immunofluorescence.
127. The method of any one of embodiments 111-126, wherein sample is a formalin-fixed paraffin-embedded sample.
128. The method of any one of embodiments 111-127, wherein the sample comprises a cell.
129. The method of any one of embodiments 111-128, wherein the sample comprises a tissue sample.
130. The method of any one of embodiments 111-129, wherein the sample comprises immune cells.
131. The method of embodiment 131, wherein the immune cells are selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and peripheral blood mononuclear cells (PBMCs)
132. The method of any one of embodiments 111-131, wherein the sample comprises a tissue or cells associated with a disease or disorder.
133. The method of any one of embodiments 111-132, wherein the disease or disorder is a cancer, an autoimmune disorder, an inflammatory disorder, or an infection.
134. The method of embodiment 132 or 133, wherein the disease or disorder is chosen from non-viral cancers, virus-associated cancers, cancers associated with HBV infection, cancers associated with Epstein-Barr virus (EBV) infection, cancers associated with polyomavirus infection, erythema nodosum leprosum (ENL), autoimmune diseases, autoimmune inflammation, autoimmune thyroid diseases, B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, B-cell leukemias, myelodysplastic syndromes, solid phase cancer, herpes viral infections, and/or rejection of transplanted tissues or organs.
135. The isolated antibody or antigen binding fragment thereof of any one of embodiments 1 to 76, for use in a method of associating presence or abundance of AXL with a location of interest of a tissue sample.
136. The isolated antibody or antigen binding fragment thereof of any one of embodiments 1 to 76, for use in a method of detecting AXL in a tissue sample.
137. The method of any of embodiments 111-134, wherein the method comprises generating a nucleic acid molecule comprising all or a portion of the sequence of the oligonucleotide or a complement thereof.
138. The isolated antibody or antigen binding fragment thereof of any one of embodiments 1 to 76, for use in the construction of a protein library.
139. The isolated antibody or antigen binding fragment thereof of embodiment 138, wherein the construction of a protein library comprises sequencing.
140. The isolated antibody or antigen binding fragment thereof of embodiment 138, wherein the construction of a protein library comprises the use of flow cytometry.

### EXAMPLES

The examples set forth below illustrate certain embodiments and do not limit the technology.

### Example 1. Generation Anti-HUMAN AXL Antibody Expressing Hybridomas

Briefly, mice were immunized with a human AXL immunogen, hybridomas were formed using standard protocols to fuse myeloma cells with spleens, and lymph node cells were drained and harvested. Successful fusions were selected into HAT medium, and cloned into approximately one cell per well in microtiter plates, after which culture supernatants were tested against human AXL-expressing transfected cells by flow cytometry. Wells were selected by assessment of staining profiles and then sub-cultured into larger vessels and sub-cloned. Hybridoma sub-clones were further characterized by flow cytometry using human AXL-transfected cells. Candidate clones expressing exemplary anti-human AXL antibodies were selected and screened using various methods, including by flow cytometry against human blood cells divided into distinct subsets (e.g., lymphocytes, monocytes, and the like), and against one or more cell lines generated from diseased.

### Example 2. Sequencing of Exemplary Anti-HUMAN AXL Antibody Variable Regions

This Example describes the sequencing of exemplary anti-human AXL antibodies generated in Example 1 above.

Cells from anti-human AXL hybridoma cell lines described in Example 1 above were grown in standard mammalian tissue culture media. Total RNA was isolated from hybridoma cells from various clones expressing anti-human AXL monoclonal antibodies using a procedure based on the RNeasy Mini Kit (Qiagen). Briefly, RNA was used to generate a first strand of cDNA of the light chain and heavy chain variable domains. Both light chain and heavy chain variable domain cDNAs were amplified by a 5′-RACE technique, and positive clones were prepared by PCR and then subjected to DNA sequencing.

Amino acid sequences of the individual variable domains (CDRs and Framework regions), including the CDR1, CDR2, and CDR3 regions, for both the heavy and light chains for six different antibodies, designated AB 1-6 (also referred to herein as antibodies 1-6). The various heavy and light chain CDR sequences are shown in Table E1, below.

TABLE E1
CDR Sequences and SEQ ID NOs. of VH and VL domains of exemplary
anti-HUMAN AXL antibodies.

| SEQ | | AB | |
|---|---|---|---|
| ID NO | CDR Type | Number(s) | Amino Acid Sequence |
| 24 | CDRH1 | 1, 2, 4 | SAYSWH |
| 25 | CDRH1 | 3, 5 | NYGMN |
| 26 | CDRH1 | 6 | DYYMY |
| 27 | CDRH2 | 1, 2, 4 | YIHYSGSTNYNPSLKS |
| 28 | CDRH2 | 3 | WINTYIGEPTYADDFKG |
| 29 | CDRH2 | 5 | WINTYTGEPTYADDFKG |
| 30 | CDRH2 | 6 | YISNGGGSTYYPDTVKG |
| 31 | CDRH3 | 1, 2, 4 | SLWFYAMDY |
| 32 | CDRH3 | 3 | GTTFYAMDY |
| 33 | CDRH3 | 5 | GDRFYAMDY |
| 34 | CDRH3 | 6 | LHFYAMDY |
| 35 | CDRL1 | 1, 4 | RASKSISKYLA |
| 36 | CDRL1 | 2 | HASQNINVWLN |
| 37 | CDRL1 | 3, 5 | SASSSISSNYLH |
| 38 | CDRL1 | 6 | KASQDVSTAVA |
| 39 | CDRL2 | 1 | SGSTLQS |
| 40 | CDRL2 | 2 | KASNLHT |
| 41 | CDRL2 | 3, 5 | RTSNLAS |
| 42 | CDRL2 | 4 | SGSTLHS |
| 43 | CDRL2 | 6 | WASTRHT |
| 44 | CDRL3 | 1, 4 | QQHNEYPWT |
| 45 | CDRL3 | 2 | QQGQSYPLT |
| 46 | CDRL3 | 3 | QQGSSILT |
| 47 | CDRL3 | 5 | QQGSSIFT |
| 48 | CDRL3 | 6 | QQHYNTPFT |

### Example 3. Detection of HUMAN AXL Expressing Cells Using Exemplary Anti-HUMAN AXL Antibodies

This Example describes the ability of exemplary generated anti-human AXL antibodies to detect target cells expressing human AXL, assessed by flow cytometry.

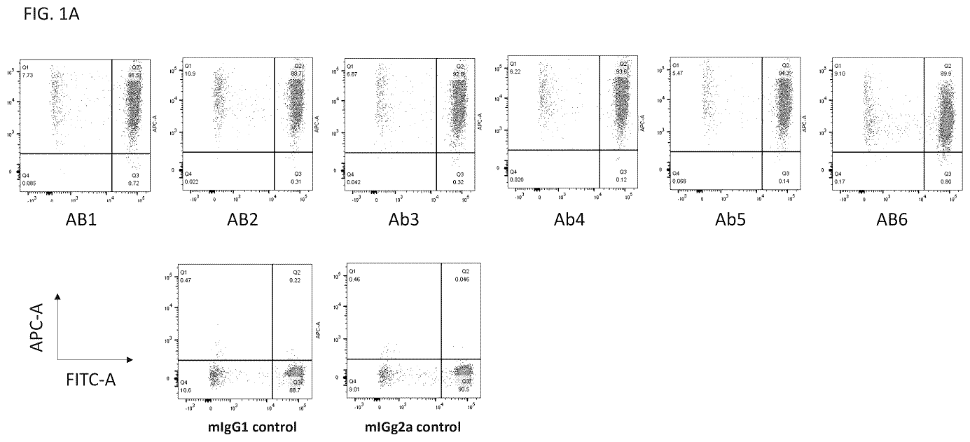

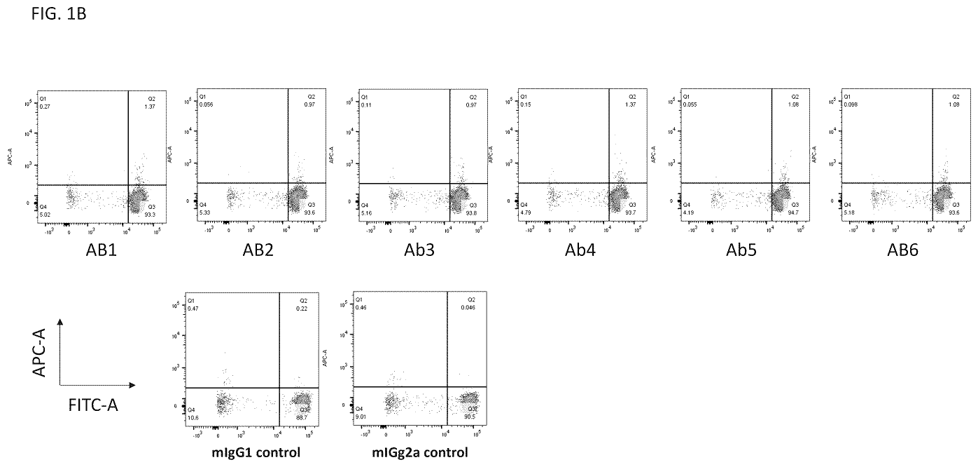

In a first experiment, exemplary anti-human AXL antibodies were assessed on an IL-3 dependent murine pro B cell line (BA/F3, RIKEN CELL BANK, Cat #RCB4476), transfected with Human AXLΔcyto-IG (Ba/F3 cells were retrovirally transduced with human AXL lacking the cytoplasmic domain and GFP). Transfected BA/F3 cells were stained with lug of exemplary antibodies AB1-AB6, or isotype controls (mouse IgG1 for clone AB1, 2, 3, 4 and 5; mouse IgG2a for clone AB6), and allowed to incubate for 15 minutes. Cells were then washed twice with FACS wash buffer and stained with anti-mouse IgG-APC secondary antibody for 15 minutes. Cells were washed with FACS buffer and analyzed on a BD Canto II and corresponding data was analyzed by FlowJo. As shown in FIGS. 1A-1B, all exemplary tested anti-human AXL antibodies were capable of staining Human AXL transfected cells (FIG. 1A), but not mGypa (FIG. 1B) transfected cells (vector control cell line).

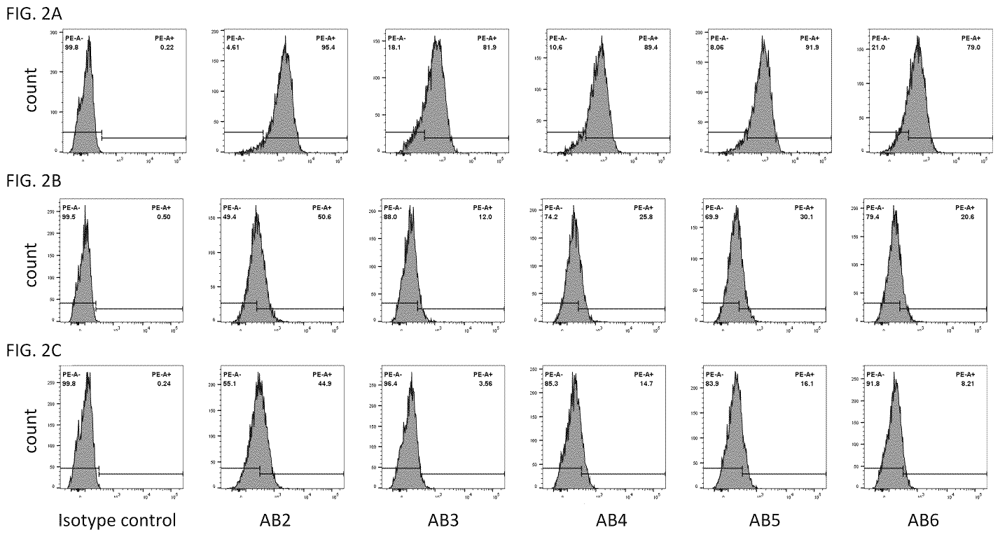

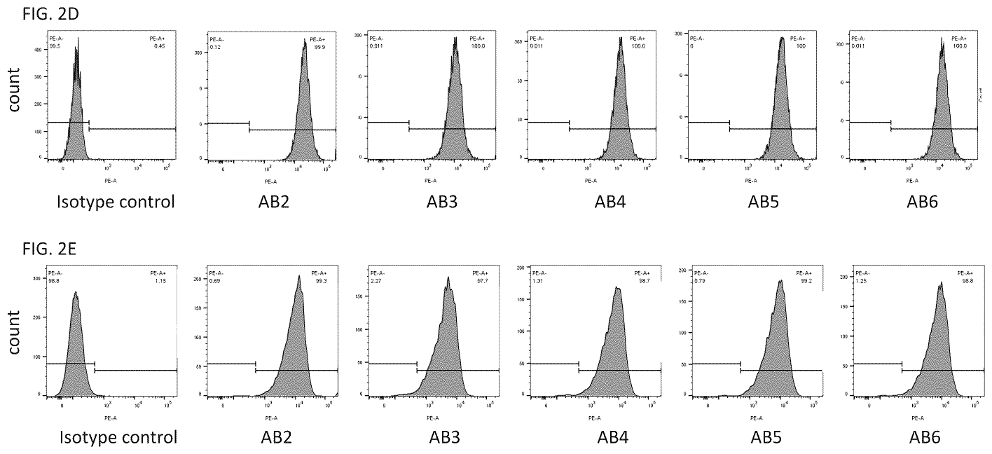

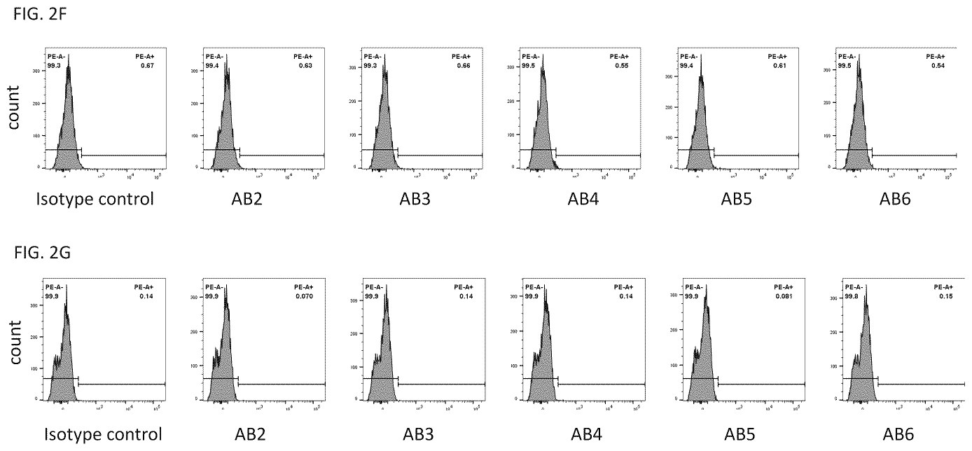

In another experiment, carcinoma cell lines A-431 (CRL-1555™, Epidermoid Carcinoma, ATCC), A-549 (ATCC® CCL-185™, Lung carcinoma, ATCC), HeLa (ATCC® CCL-2™, Cervix Adenocarcinoma, ATCC), MDA-MB-231 (HTB-26, breast, Adenocarcinoma, ATCC), and NCI-H1299 (ATCC® CRL-5803™, non-small cell lung carcinoma, ATCC), were grown following manufacturer's protocols. Once cells reached 80% confluency, cells were detached from the flask using 0.25% Trypsin-0.1% EDTA and suspended in cell staining buffer. 0.25 μg of exemplary generated anti-human AXL antibodies AB2-AB6 PE conjugates were added, and allowed to incubate for 15 minutes. Cells were washed with FACS buffer and analyzed on a BD Canto II flow cytometer. As shown in FIGS. 2A-2E, all exemplary tested anti-human AXL antibodies were capable of staining A-431 (FIG. 2A), A-549 (FIG. 2B), HeLa (FIG. 2C), MDA-MB-231 (FIG. 2D) and NCI-H1299 (FIG. 2E) cells, as compared to isotype control. Cell lines that are not known to express the human AXL receptor tyrosine kinase, TH-29 (ATCC® HTB-38™ from ATCC) and Jurkat (Clone E6-1, ATCC TIB-152 from ATCC) were grown following manufacturer's protocols, and staining protocol was conducted similarly to above. Exemplary tested anti-human AXL antibodies AB2-AB6 PE conjugates demonstrated negative surface staining, TH-29 (FIG. 2F) and Jurkat (FIG. 2G), respectively.

These results demonstrate the ability of exemplary generated anti-human AXL antibodies to specifically bind the cognate receptor human AXL receptor tyrosine kinase, with minimal to no non-specific binding.

### Example 4. Assessment of the Specific Binding of Exemplary Anti-Human AXL Antibodies to Human AXL Receptor Tyrosine Kinase

This Example describes assessment of the ability of exemplary generated anti-AXL antibodies to specifically bind to the AXL receptor on a target cell.

Briefly, BA/F3 cells were transfected with huAXLΔcyto-IG similarly to Example 3 above. hAXL recombinant proteins (Sino Biological, Catalog #10279-H08H) were incubated in separate tubes with exemplary anti-human antibodies AB2-AB6 conjugated to PE, or AB1 PU, for 15 minutes at room temperature. PE conjugated mIgG1 and mIgG2a, or PU-IgG1 were used as controls. After incubation, 2×10⁶/100 μL of huAXLΔcyto-IG transfected BA/F3 cells were added to each tube, and cells were acquired on a BD Canto II and corresponding data was analyzed by FlowJo.

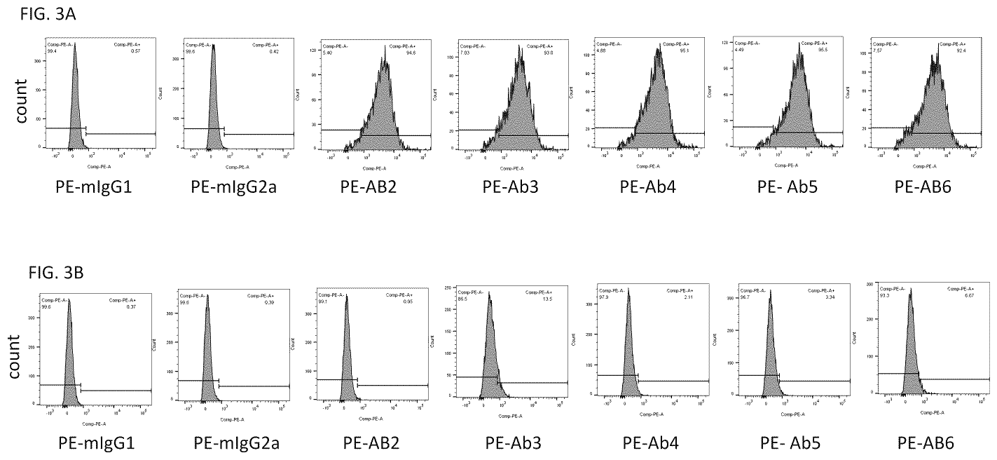

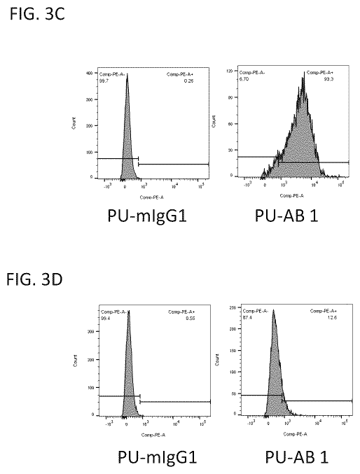

As shown in FIGS. 3A-3D, exemplary antibodies AB2-AB6 (FIG. 3B) and AB1 (FIG. 3D) are all neutralized upon addition of human AXL recombinant protein, as compared to the absence of the recombinant protein for AB2-AB6 (FIG. 3A) and AB1 (FIG. 3C) respectively.

These results suggest that exemplary tested antibodies specifically bind to the AXL expressing cell.

### Example 5. Assessment of Functional Activity of Exemplary Anti-Human AXL Antibodies

This Example describes assessment of the functional activity of exemplary anti-human AXL antibodies by evaluation of the ability to block GAS6 induced AKT and ERK phosphorylation.

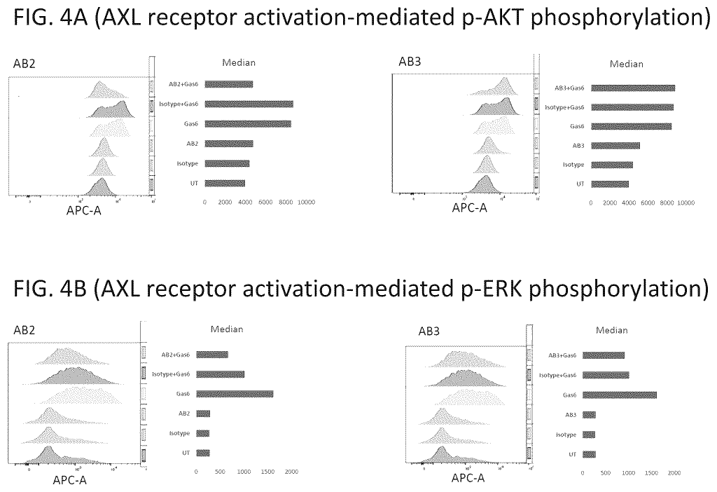

For phosphorylation assays, H1299 cells, which are capable of Gas6 inducible human AXL activation, were grown in RPMI-1640 medium according to manufacturer's specifications. The cells were seeded at one million per T75 flask (T75) and sub-cultured every two or three days. After one or two passages, the cells were seeded at an appropriate density for a T75 flask. The cells were placed in a 37° C. incubator and allowed to grow 24 hours. The cells were observed under a light microscope the following day. When the cells were approximately 80% confluent, the media was removed and replaced with serum-free media (basal RPMI-1640 media without added FBS). After 48 hours, the cells were trypsinized with 2-3 mL of trypsin/EDTA solution to the flask. Once the cells detached (rounding up at the edge), the flask was tapped to dislodge the cells. The trypsin was neutralized with 6-8 ml of culture medium (containing 10% FBS) and the cells were transferred to a 50 mL sterile conical tube. The tube was filled with 1× sterile PBS to 50 mL, and was centrifuged at 1500 rpm for 5 min. The supernatant was discarded and the cells were washed with 1× sterile PBS two times (50 ml PBS, centrifuged at 1500 rpm for 5 min). The cells were resuspended in serum-free RPMI-1640 in 15 ml conical tubes at a density of 0.5-1 million cells/test/tube. 10 μg of exemplary antibodies AB2 and AB3, or isotype controls MOPC-21 and MOPC-173 were added to the tubes. The 15 ml conicals containing the cells and exemplary antibodies were placed in the 37° C. water bath for 60 minutes. Phosphorylation was induced by adding Gas6 (Acrobiosystems.com, Cat. No. GA6-H5249) at a final concentration of 50 nM for 5-10 minutes to the rested cells. The cells were fixed by adding an equal volume of pre-warmed Fixation Buffer (BioLegend Cat #420801) to each 15 ml conical in the 37° C. water bath for 15 minutes, then the cells were permeabilized by adding 0.3 mL/1×10⁶ cells of True-Phos™ Perm Buffer (BioLegend Cat #425401) to each 15 mL conical while vortexing, and incubated at −20° C. for 60 minutes. Cells were stained with an APC-anti-Phospho-AKT1 (Ser473) Monoclonal Antibody (clone SDRNR, Thermofisher, Cat #17-9715-42), or a PE-anti-ERK1/2 Phospho (Thr202/Tyr204) Antibody (clone 4B11B69, Biolegend, Cat #369506). The cells were acquired on a BD Canto II and corresponding data was analyzed by FlowJo. Results are shown in FIGS. 4A-4B, and quantification of results are shown in Table E2 and E3.

As shown in FIGS. 4A and 4B and Table E2 and E3, exemplary antibody AB2 was surprisingly capable of blocking Gas6-induced AKT phosphorylation (FIG. 4A and Table E2), and Gas6-induced ERK phosphorylation (FIG. 4B and Table E3), while exemplary antibody AB3 failed to block the AXL receptor activation, thereby allowing for very distinct applications for each of AB2 and AB3, including for example, therapeutic applications for AB2 and diagnostic and/or research tool applications for AB2 and AB3.

TABLE E2
Analysis of Gas6 induced AKT phosphorylation in H1299 cells

| | |
|---|---|
| | APC-A |
| Median: APC-A | Geometric mean |

AB2

| | | |
|---|---|---|
| p-AKT APC + AB2 + Gas6 | 4760 | 5219 |
| p-AKT APC + isotype | 9118 | 8385 |
| control + Gas6 | | |
| p-AKT APC + Gas6 | 8514 | 8010 |
| p-AKT APC + AB2 | 4771 | 4725 |
| p-AKT APC + isotype | 4427 | 4377 |
| control | | |
| p-AKT APC untreated | 3887 | 3800 |

AB3

| | | |
|---|---|---|
| p-AKT APC + AB3 + Gas6 | 10878 | 9163 |
| p-AKT APC + isotype | 9118 | 8385 |
| control + Gas6 | | |
| p-AKT APC + Gas6 | 8514 | 8010 |
| p-AKT APC + AB3 | 5168 | 5368 |
| p-AKT APC + isotype | 4427 | 4377 |
| control | | |
| p-AKT APC untreated | 3887 | 3800 |

TABLE E3
Analysis of Gas6 induced ERK phosphorylation in H1299 cells

| | |
|---|---|
| | APC-A |
| Median: APC-A | Geometric mean |

AB2

| | | |
|---|---|---|
| p-ERK A647 + AB2 + Gas6 | 669 | 969 |
| p-ERK A647 + isotype | 1016 | 1325 |
| control + Gas6 | | |
| p-ERK A647 + Gas6 | 1620 | 1965 |
| p-ERK A647 + AB2 | 292 | 830 |
| p-ERK A647 + isotype | 274 | 965 |
| control | | |
| p-ERK A647 untreated | 285 | 1037 |

AB3

| | | |
|---|---|---|
| p-ERK A647 + AB3 + Gas6 | 919 | 1291 |
| p-ERK A647 + isotype | 1016 | 1325 |
| control + Gas6 | | |
| p-ERK A647 + Gas6 | 1620 | 1695 |
| p-ERK A647 + AB3 | 285 | 856 |
| p-ERK A647 + isotype | 274 | 965 |
| control | | |
| p-ERK A647 untreated | 285 | 1037 |

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

SEQUENCE TABLE

| SEQ | | |
|---|---|---|
| ID NO | SEQUENCE | DESCRIPTION |
| 1 | DVQLQESGPDLVKPSQSLSLTCTVTGYSITSAYSWHWIRQFPGNKLEWMGYI | VH amino acid |
| | HYSGSTNYNPSLKSRISITRDTSKNQFFLQLNSVTTEDTATYYCARSLWFYAMD | |
| | YWGQGTSVTVSS | |
| 2 | QIQLVQSGPELKKPGETVKISCKASGYTFTNYGMNWVKQAPGKGLKWMGW | VH amino acid |
| | INTYIGEPTYADDFKGRFAFSLETSASTAYLQINNLKNEDMGTYFCARGTTFYA | |
| | MDYWGQGTSVTVSS | |
| 3 | DVQLQESGPDLVKPSQSLSLTCTVTGYSITSAYSWHWIRQFPGNKLEWMGYI | VH amino acid |
| | HYSGSTNYNPSLKSRISITRDTSKNQFFLQLSSVTTEDTATYYCARSLWFYAMD | |
| | YWGQGTSVTVSS | |
| 4 | QIQLVQSGPELKKPGETVKISCKASGYTFTNYGMNWVKQAPGKGLKWMGW | VH amino acid |
| | INTYTGEPTYADDFKGRFAFSLETSASTAYLQINNLKNEDMATYFCARGDRFY | |
| | AMDYWGQGTSVTVSS | |
| 5 | EVNLVESGGGLVQPGGSLKLSCATSGFTFSDYYMYWVRQTPEKRLEWVAYIS | VH amino acid |
| | NGGGSTYYPDTVKGRFTISRDNAKNTLYLQMSRLKSEDTAMYYCARLHFYAM | |
| | DYWGQGTSVTVSS | |
| 6 | DVQITQSPSYLAASPGETITINCRASKSISKYLAWYQEKPGKTNKLLIYSGSTLQS | VL amino acid |
| | GIPSRFSGSGSGTDFTLTISSLEPEDFAMYYCQQHNEYPWTFGGGTKLEIK | |
| 7 | DIQMNQSPSSLSASLGDTITITCHASQNINVWLNWYQQKPGNIPKLLIYKASN | VL amino acid |
| | LHTGVPSRFSGSGSGTGFTLTISSLQPEDIATYYCQQGQSYPLTFGGGTKLEIK | |
| 8 | EIVLTQSPTTMAASPGEKITITCSASSSISSNYLHWYQQKPGFSPKLLIYRTSNLA | VL amino acid |
| | SGVPARFSGSGSGTSYSLTIGTMEAEDVATYYCQQGSSILTFGAGTKLELK | |
| 9 | DVQITQSPSYLAASPGETITIKCRASKSISKYLAWYQEKPGKTNKLLIYSGSTLHS | VL amino acid |
| | GIPSRFSGSGSGTDFTLTISSLEPEDFAMYYCQQHNEYPWTFGGGTKLEIK | |
| 10 | EIVLTQSPTTMAASPGEKITITCSASSSISSNYLHWYQQKPGFSPKLLIYRTSNLA | VL amino acid |
| | SGVPTRFSGSGSGTSYSLTIGTMEAEDVATYYCQQGSSIFTFGSGTKLEIK | |
| 11 | DIVMTQSHKFMSTSVGDRVSITCKASQDVSTAVAWYQQKPGQSPKLLIYWA | VL amino acid |
| | STRHTGVPDRFTGSGSGTDYTLTISSVQAEDLALYYCQQHYNTPFTFGSGTKLE | |
| | IK | |
| 12 | ATGAGAGTGCTGATTCTTTTGTGCCTGTTCACAGCCTTTCCTGGTATCCTGT | VH nucleic acid |
| | CTGATGTGCAGCTTCAGGAGTCAGGACCTGACCTGGTGAAACCTTCTCAGT | |
| | CACTTTCACTCACCTGCACTGTCACTGGCTACTCCATCACCAGTGCTTATAG | |
| | CTGGCACTGGATCCGGCAGTTTCCAGGAAACAAACTGGAATGGATGGGCT | |
| | ACATACACTACAGTGGTAGCACTAACTACAACCCATCTCTCAAAAGTCGAA | |
| | TCTCTATCACTCGAGACACATCCAAGAACCAGTTCTTCCTGCAGTTGAATTC | |
| | TGTGACTACTGAGGACACAGCCACATATTACTGTGCAAGAAGCCTATGGTT | |
| | CTATGCTATGGACTACTGGGGTCAAGGAACCTCAGTCACCGTCTCCTCA | |
| 13 | ATGAGAGTGCTGATTCTTTTGTGCCTGTTCACAGCCTTTCCTGGTATCCTGT | VH nucleic acid |
| | CTGATGTGCAGCTTCAGGAGTCAGGACCTGACCTGGTGAAACCTTCTCAGT | |
| | CACTTTCACTCACCTGCACTGTCACTGGCTACTCCATCACCAGTGCTTATAG | |
| | CTGGCACTGGATCCGGCAGTTTCCAGGAAACAAACTGGAATGGATGGGCT | |
| | ACATACACTACAGTGGTAGCACTAACTACAACCCATCTCTCAAAAGTCGAA | |
| | TCTCTATCACTCGAGACACATCCAAGAACCAGTTCTTCCTGCAGTTGAATTC | |
| | TGTGACTACTGAGGACACAGCCACATATTACTGTGCAAGAAGCCTATGGTT | |
| | CTATGCTATGGACTACTGGGGTCAAGGAACCTCAGTCACCGTCTCCTCA | |
| 14 | ATGGATTGGCTGTGGAACTTGCTATTCCTGATGGCAGCTGCCCAAAGTGCC | VH nucleic acid |
| | CAAGCACAGATCCAGTTGGTGCAGTCTGGACCTGAGCTGAAGAAGCCTGG | |
| | AGAGACAGTCAAGATCTCCTGCAAGGCTTCTGGATATACCTTCACAAACTA | |
| | TGGAATGAACTGGGTGAAGCAGGCTCCAGGAAAGGGTTTGAAGTGGATG | |
| | GGCTGGATAAACACCTACATTGGAGAGCCAACATATGCTGATGACTTCAA | |
| | GGGACGGTTTGCCTTCTCTTTGGAAACCTCTGCCAGCACTGCCTATTTGCA | |
| | GATCAACAACCTCAAAAATGAGGACATGGGTACATATTTCTGTGCAAGGG | |
| | GGACTACGTTCTATGCTATGGACTACTGGGGTCAAGGAACCTCAGTCACC | |
| | GTCTCCTCA | |
| 15 | ATGAGAGTGCTGATTCTTTTGTGCCTGTTCACAGCCTTTCCTGGTATCCTGT | VH nucleic acid |
| | CTGATGTGCAGCTTCAGGAGTCAGGACCTGACCTGGTGAAACCTTCTCAGT | |
| | CACTTTCACTCACCTGCACTGTCACTGGCTACTCCATCACCAGTGCTTATAG | |
| | CTGGCACTGGATCCGGCAGTTTCCAGGAAACAAACTGGAATGGATGGGCT | |
| | ACATACACTACAGTGGTAGCACTAACTACAACCCATCTCTCAAAAGTCGAA | |
| | TCTCTATCACTCGAGACACATCCAAGAACCAGTTCTTCCTGCAGTTGAGTTC | |
| | TGTGACTACTGAGGACACAGCCACATATTACTGTGCAAGAAGCCTATGGTT | |
| | CTATGCTATGGACTACTGGGGTCAAGGAACCTCAGTCACCGTCTCCTCA | |
| 16 | ATGGATTGGCTGTGGAACTTGCTATTCCTGATGGCAGCTGCCCAAAGTGCC | VH nucleic acid |
| | CAAGCACAGATCCAGTTGGTGCAGTCTGGACCTGAGCTGAAGAAGCCTGG | |
| | AGAGACAGTCAAGATCTCCTGCAAGGCTTCTGGGTATACCTTCACAAACTA | |
| | TGGAATGAACTGGGTGAAGCAGGCTCCAGGAAAGGGTTTAAAGTGGATG | |
| | GGCTGGATAAACACCTACACTGGAGAGCCAACATATGCTGATGACTTCAA | |
| | GGGACGGTTTGCCTTCTCTTTGGAAACCTCTGCCAGCACTGCCTATTTGCA | |
| | GATCAACAACCTCAAAAATGAGGACATGGCTACATATTTCTGTGCAAGAG | |
| | GTGATAGGTTCTATGCTATGGACTACTGGGGTCAAGGAACCTCAGTCACC | |
| | GTCTCCTCA | |
| 17 | ATGAACTTGGGGCTCAGCTTGATTTTCCTTGTCCTTGTTTTAAAAGGTGTCC | VH nucleic acid |
| | AGTGTGAAGTGAACCTGGTGGAGTCTGGGGGAGGCTTAGTGCAGCCTGG | |
| | AGGGTCCCTGAAACTCTCCTGTGCAACCTCTGGATTCACTTTCAGTGACTAT | |
| | TACATGTATTGGGTTCGCCAGACTCCAGAGAAGAGGCTGGAGTGGGTCGC | |
| | ATACATTAGTAATGGTGGTGGTAGCACCTATTATCCAGACACTGTAAAGG | |
| | GCCGATTCACCATCTCCAGAGACAATGCCAAGAACACCCTGTACCTGCAAA | |
| | TGAGCCGTCTGAAGTCTGAGGACACAGCCATGTATTACTGTGCAAGACTTC | |
| | ATTTCTATGCTATGGACTACTGGGGTCAAGGAACCTCAGTCACCGTCTCCT | |
| | CA | |
| 18 | ATGAGGTTCCAGGTTCAGGTTCTGGGGCTCCTTCTGCTCTGGATATCAGGT | VL nucleic acid |
| | GCCCAGTGTGATGTCCAGATAACCCAGTCTCCATCTTATCTTGCTGCATCTC | |
| | CTGGAGAAACCATTACTATTAATTGCAGGGCAAGTAAGAGCATTAGCAAA | |
| | TATTTAGCCTGGTATCAAGAGAAACCTGGGAAAACTAATAAGCTTCTTATC | |
| | TACTCTGGATCCACTTTGCAATCTGGAATTCCATCAAGGTTCAGTGGCAGT | |
| | GGATCTGGTACAGATTTCACTCTCACCATCAGTAGCCTGGAGCCTGAAGAT | |
| | TTTGCAATGTATTACTGTCAACAGCATAATGAATACCCGTGGACGTTCGGT | |
| | GGAGGCACCAAGCTGGAAATCAAA | |
| 19 | ATGAGGGTCCTTGCTGAGCTCCTGGGGCTGCTGCTGTTCTGCTTTTTAGGT | VL nucleic acid |
| | GTGAGATGTGACATCCAGATGAACCAGTCTCCATCCAGTCTGTCTGCATCC | |
| | CTTGGAGACACAATTACCATCACTTGCCATGCCAGTCAGAACATTAATGTT | |
| | TGGTTAAACTGGTACCAGCAGAAACCAGGAAATATTCCTAAACTATTGATC | |
| | TATAAGGCTTCCAACTTGCACACAGGCGTCCCATCAAGGTTTAGTGGCAGT | |
| | GGATCTGGAACAGGTTTCACATTAACCATCAGCAGCCTGCAGCCTGAAGA | |
| | CATTGCCACTTACTACTGTCAACAGGGTCAAAGTTATCCTCTGACGTTCGG | |
| | TGGAGGCACCAAGCTGGAAATCAAA | |
| 20 | ATGCAGATTATCAGCTTGCTGCTAATCAGTGTCACAGTCATAGTGTCTAAT | VL nucleic acid |
| | GGAGAAATTGTGCTCACCCAGTCTCCAACCACCATGGCTGCATCTCCCGGG | |
| | GAGAAGATCACTATCACCTGCAGTGCCAGCTCAAGTATAAGTTCCAATTAC | |
| | TTGCATTGGTATCAGCAGAAGCCAGGATTCTCCCCTAAACTCTTGATTTATA | |
| | GGACATCCAATCTGGCTTCTGGAGTCCCAGCTCGCTTCAGTGGCAGTGGG | |
| | TCTGGGACCTCTTACTCTCTCACAATTGGCACCATGGAGGCTGAAGATGTT | |
| | GCCACTTACTACTGCCAGCAGGGTAGTAGTATACTCACGTTCGGTGCTGG | |
| | GACCAAGCTGGAGCTGAAA | |
| 21 | ATGAGGTTCCAGGTTCAGGTTCTGGGGCTCCTTCTGCTCTGGATATCAGGT | VL nucleic acid |
| | GCCCAGTGTGATGTCCAGATAACCCAGTCTCCATCCTATCTTGCTGCATCTC | |
| | CTGGAGAAACCATTACTATTAAATGCAGGGCAAGTAAGAGCATTAGTAAA | |
| | TATTTAGCCTGGTATCAAGAGAAACCTGGGAAAACTAATAAGCTTCTTATC | |
| | TACTCTGGATCCACTTTGCATTCTGGAATTCCATCAAGGTTCAGTGGCAGT | |
| | GGATCTGGTACAGATTTCACTCTCACCATCAGTAGCCTGGAGCCTGAAGAT | |
| | TTTGCAATGTATTACTGTCAACAGCATAATGAATACCCGTGGACGTTCGGT | |
| | GGAGGCACCAAGCTGGAAATCAAA | |
| 22 | ATGCAGATTATCAGCTTGCTGCTAATCAGTGTCACAGTCATAGTGTCTAAT | VL nucleic acid |
| | GGAGAAATTGTGCTCACCCAGTCTCCAACCACCATGGCTGCATCTCCCGGG | |
| | GAGAAGATCACTATCACCTGCAGTGCCAGCTCAAGTATAAGTTCCAATTAC | |
| | TTGCATTGGTATCAGCAGAAGCCAGGATTCTCCCCTAAACTCTTGATTTATA | |
| | GGACATCCAATCTGGCTTCTGGAGTCCCAACTCGCTTCAGTGGCAGTGGGT | |
| | CTGGGACCTCTTACTCTCTCACAATTGGCACCATGGAGGCTGAAGATGTTG | |
| | CCACTTACTACTGCCAGCAGGGTAGTAGTATATTCACGTTCGGCTCGGGGA | |
| | CAAAGTTGGAAATAAAA | |
| 23 | ATGGAGTCACAGATTCAGGCATTTGTATTCGTGTTTCTCTGGTTGTCTGGT | VL nucleic acid |
| | GTTGACGGAGACATTGTGATGACCCAGTCTCACAAATTCATGTCCACATCA | |
| | GTAGGAGACAGGGTCAGCATCACCTGCAAGGCCAGTCAGGATGTGAGTA | |
| | CTGCTGTAGCCTGGTATCAACAAAAACCAGGGCAATCTCCTAAACTACTGA | |
| | TTTACTGGGCATCCACCCGGCACACTGGAGTCCCTGATCGCTTCACAGGCA | |
| | GTGGATCTGGGACAGATTATACTCTCACCATCAGCAGTGTGCAGGCTGAA | |
| | GACCTGGCACTTTATTACTGTCAGCAACATTATAACACTCCATTCACGTTCG | |
| | GCTCGGGGACAAAGTTGGAAATAAAA | |
| 24 | SAYSWH | CDRH1 |
| 25 | NYGMN | CDRH1 |
| 26 | DYYMY | CDRH1 |
| 27 | YIHYSGSTNYNPSLKS | CDRH2 |
| 28 | WINTYIGEPTYADDFKG | CDRH2 |
| 29 | WINTYTGEPTYADDFKG | CDRH2 |
| 30 | YISNGGGSTYYPDTVKG | CDRH2 |
| 31 | SLWFYAMDY | CDRH3 |
| 32 | GTTFYAMDY | CDRH3 |
| 33 | GDRFYAMDY | CDRH3 |
| 34 | LHFYAMDY | CDRH3 |
| 35 | RASKSISKYLA | CDRL1 |
| 36 | HASQNINVWLN | CDRL1 |
| 37 | SASSSISSNYLH | CDRL1 |
| 38 | KASQDVSTAVA | CDRL1 |
| 39 | SGSTLQS | CDRL2 |
| 40 | KASNLHT | CDRL2 |
| 41 | RTSNLAS | CDRL2 |
| 42 | SGSTLHS | CDRL2 |
| 43 | WASTRHT | CDRL2 |
| 44 | QQHNEYPWT | CDRL3 |
| 45 | QQGQSYPLT | CDRL3 |
| 46 | QQGSSILT | CDRL3 |
| 47 | QQGSSIFT | CDRL3 |
| 48 | QQHYNTPFT | CDRL3 |
| 49 | MRVLILLCLFTAFPGILS | VH signal |
| | | sequence |
| 50 | MDWLWNLLFLMAAAQSAQA | VH signal |
| | | sequence |
| 51 | MNLGLSLIFLVLVLKGVQC | VH signal |
| | | sequence |
| 52 | MRFQVQVLGLLLLWISGAQC | VL signal |
| | | sequence |
| 53 | MRVLAELLGLLLFCFLGVRC | VL signal |
| | | sequence |
| 54 | MQIISLLLISVTVIVSNG | VL signal |
| | | sequence |
| 55 | MESQIQAFVFVFLWLSGVDG | VL signal |
| | | sequence |
| 56 | MAWRCPRMGRVPLAWCLALCGWACMAPRGTQAEESPFVGNPGNITGAR | human AXL |
| | GLTGTLRCQLQV | protein |
| | QGEPPEVHWLRDGQILELADSTQTQVPLGEDEQDDWIVVSQLRITSLQLSDT | |
| | GQYQCLVF | |
| | LGHQTFVSQPGYVGLEGLPYFLEEPEDRTVAANTPFNLSCQAQGPPEPVDLL | |
| | WLQDAVPL | |
| | ATAPGHGPQRSLHVPGLNKTSSFSCEAHNAKGVTTSRTATITVLPQQPRNLHL | |
| | VSRQPTE | |
| | LEVAWTPGLSGIYPLTHCTLQAVLSDDGMGIQAGEPDPPEEPLTSQASVPPH | |
| | QLRLGSLH | |
| | PHTPYHIRVACTSSQGPSSWTHWLPVETPEGVPLGPPENISATRNGSQAFVH | |
| | WQEPRAPL | |
| | QGTLLGYRLAYQGQDTPEVLMDIGLRQEVTLELQGDGSVSNLTVCVAAYTAA | |
| | GDGPWSLP | |
| | VPLEAWRPGQAQPVHQLVKEPSTPAFSWPWWYVLLGAVVAAACVLILALFL | |
| | VHRRKKETR | |
| | YGEVFEPTVERGELVVRYRVRKSYSRRTTEATLNSLGISEELKEKLRDVMVDRH | |
| | KVALGK | |
| | TLGEGEFGAVMEGQLNQDDSILKVAVKTMKIAICTRSELEDELSEAVCMKEFD | |
| | HPNVMRL | |
| | IGVCFQGSERESFPAPVVILPFMKHGDLHSFLLYSRLGDQPVYLPTQMLVKFM | |
| | ADIASGM | |
| | EYLSTKRFIHRDLAARNCMLNENMSVCVADFGLSKKIYNGDYYRQGRIAKMP | |
| | VKWIAIES | |
| | LADRVYTSKSDVWSFGVTMWEIATRGQTPYPGVENSEIYDYLRQGNRLKQP | |
| | ADCLDGLYA | |
| | LMSRCWELNPQDRPSFTELREDLENTLKALPPAQEPDEILYVNMDEGGGYPE | |
| | PPGAAGGA | |
| | DPPTQPDPKDSCSCLTAAEVHPAGRYVLCPSTTPSPAQPADRGSPAAPGQED | |
| | GA | |
| 57 | X1X2X3X4X5X6 | CDRH1 |
| | | X1 is S, N or D |
| | | X2 is A or Y |
| | | X3 is Y or G |
| | | X4 is S or M |
| | | X5 is W, N or Y |
| | | X6 is H or no |
| | | amino acid |
| 58 | X1IX3X4X5X6X7X8X9X10X11X12X13X14X15X16X17 | CDRH2 |
| | | X1 is Y or W |
| | | X3 is H, N or S |
| | | X4 is Y, T or N |
| | | X5 is S, Y or G |
| | | X6 is G, I or T |
| | | X7 is S or G |
| | | X8 is T, E or S |
| | | X9 is N, P or T |
| | | X10 is Y or T |
| | | X11 is N or Y |
| | | X12 is P or A |
| | | X13 is S or D |
| | | X14 is L, D or T |
| | | X15 is K, F or V |
| | | X16 is S or K |
| | | X17 is G or no |
| | | amino acid |
| 59 | X1X2X3FYAMDY | CDRH3 |
| | | X1 is S, G or no |
| | | amino acid |
| | | X2 is L, T or D |
| | | X3 is W, T, R or H |
| 60 | X1ASX4X5X6X7X8X9X10X11X12 | CDRL1 |
| | | X1 is R, H, S or K |
| | | X4 is K, Q or S |
| | | X5 is S, N or D |
| | | X6 is I or V |
| | | X7 is S or N |
| | | X8 is K, V, S or T |
| | | X9 is Y, V, S or T |
| | | X10 is L, Y or V |
| | | X11 is A, N or L |
| | | X12 is H or no |
| | | amino acid |
| 61 | X1X2SX4X5X6X7 | CDRL2 |
| | | X1 is S, K, R or W |
| | | X2 is G, A or T |
| | | X4 is T or N |
| | | X5 is L or R |
| | | X6 is Q, H or A |
| | | X7 is S or T |
| 62 | QQX3X4X5X6X7X8X9 | CDRL3 |
| | | X3 is H or G |
| | | X4 is N, Q, S or Y |
| | | X5 is E, S or N |
| | | X6 Y, I or T |
| | | X7 is P, L or F |
| | | X8 is W, L, T or F |
| | | X9 is T or no |
| | | amino acid |
| 63 | APELLGGPS VFLFPPKPKD TLMISRTPEV TCVVVDVSHE DPEVKFNWYV | Fc polypeptide |
| | DGVEVHNAKTKPREEQYNST YRVVSVLTVL HQDWLNGKEY KCKVSNKALP | |
| | APIEKTISKA K GQPREPQVYTLPPSREEMT KNQVSLTCLV KGFYPSDIAV | |
| | EWESNGQPEN NYKTTPPVLD SDGSFFLYSKLTVDKSRWQQ | |
| | GNVFSCSVMH EALHNHYTQK SLSLSPGK | |

What is claimed is:

1. An isolated antibody or antigen binding fragment thereof that specifically binds to AXL, wherein the isolated antibody comprises:
a) a heavy chain variable region comprising:
(i) a heavy chain complementarity determining region 1 (CDRH1) comprising a sequence set forth in SEQ ID NO:24 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:24;
(ii) a heavy chain complementarity determining region 2 (CDRH2) comprising a sequence set forth in SEQ ID NO:27 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:27; and,
(iii) a heavy chain complementarity determining region 3 (CDRH3) comprising a sequence set forth in SEQ ID NO:31 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:31; and,
b) a light chain variable region comprising:
(i) a light chain complementarity determining region 1 (CDRL1) comprising a sequence set forth in SEQ ID NO:36 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:36;
(ii) a light chain complementarity determining region 2 (CDRL2) comprising a sequence set forth in SEQ ID NO:40 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:40; and,
(iii) a light chain complementarity determining region 3 (CDRL3) comprising the sequence set forth in SEQ ID NO:45 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:45.

2. The isolated antibody or antigen binding fragment thereof of claim 1, wherein the heavy chain variable region comprises at least 90% identity to the sequence set forth in SEQ ID NO: 1.

3. The isolated antibody or antigen binding fragment thereof of claim 1, wherein the light chain variable region comprises at least 90% identity to a sequence set forth in SEQ ID NO: 7.

4. The isolated antibody or antigen binding fragment thereof of claim 1, wherein the heavy chain variable region comprises at least 90% identity to a sequence set forth in SEQ ID NO: 1, and wherein the light chain variable region comprises at least 90% identity to a sequence set forth in SEQ ID NO:7.

5. The isolated antibody or antigen binding fragment thereof of claim 1, further comprising an Fc polypeptide having at least 90% identity to a sequence set forth in SEQ ID NO: 63.

6. A pharmaceutical composition comprising the isolated antibody or antigen binding fragment thereof of any one of claims 1 to 5 and a pharmaceutically acceptable carrier.

7. A diagnostic reagent comprising the isolated antibody or antigen binding fragment thereof of any one of claims 1 to 5.

8. A kit comprising the isolated antibody or antigen binding fragment thereof of any one of claims 1 to 5 or the diagnostic reagent of claim 7.

9. An isolated nucleic acid comprising a nucleotide sequence that encodes a heavy chain variable region comprising a sequence set forth in SEQ ID NO:13 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:13.

10. An isolated nucleic acid comprising a nucleotide sequence that encodes a light chain variable region comprising a sequence set forth in SEQ ID NO: 19 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:19.

11. An expression vector comprising the nucleic acid of any one of claims 9-10.

12. An isolated host cell comprising the expression vector of claim 11.

13. An isolated antibody or antigen binding fragment thereof that specifically binds to AXL, wherein the isolated antibody comprises:
a) a heavy chain variable region comprising:
(i) a heavy chain complementarity determining region 1 (CDRH1) comprising a sequence set forth in SEQ ID NO:25 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:25;
(ii) a heavy chain complementarity determining region 2 (CDRH2) comprising a sequence set forth in SEQ ID NO:28 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:28; and,
(iii) a heavy chain complementarity determining region 3 (CDRH3) comprises a sequence set forth in SEQ ID NO:32 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:32; and,
b) a light chain variable region comprising:
(i) a light chain complementarity determining region 1 (CDRL1) a sequence set forth in SEQ ID NO: 37 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:37;
(ii) a light chain complementarity determining region 2 (CDRL2) comprising a sequence set forth in SEQ ID NO:41 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:41; and,
(iii) a light chain complementarity determining region 3 (CDRL3) comprising a sequence set forth in SEQ ID NO:46 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:46.

14. The isolated antibody or antigen binding fragment thereof of claim 13, wherein the heavy chain variable region comprises at least 90% identity to the sequence set forth in SEQ ID NO: 2.

15. The isolated antibody or antigen binding fragment thereof of claim 13, wherein the light chain variable region comprises at least 90% identity to the sequence set forth in SEQ ID NO: 8.

16. The isolated antibody or antigen binding fragment thereof of claim 13, wherein the heavy chain variable region comprises at least 90% identity to a sequence set forth in SEQ ID NO: 2, and wherein the light chain variable region comprises at least 90% identity to a sequence set forth in SEQ ID NO:8.

17. The isolated antibody or antigen binding fragment thereof of claim 13, further comprising an Fc polypeptide having at least 90% identity to a sequence of SEQ ID NO:63.

18. A pharmaceutical composition comprising the isolated antibody or antigen binding fragment thereof of any one of claims 13-17 and a pharmaceutically acceptable carrier.

19. A diagnostic reagent comprising the isolated antibody or antigen binding fragment thereof of any one of claims 13-17.

20. A kit comprising the isolated antibody or antigen binding fragment thereof of any one of claims 13-17 or the diagnostic reagent of claim 19.

21. An isolated nucleic acid comprising a nucleotide sequence that encodes a heavy chain variable region comprising a sequence set forth in SEQ ID NO:14 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:14.

22. An isolated nucleic acid comprising a nucleotide sequence that encodes a light chain variable region comprising a sequence set forth in SEQ ID NO:20 or a sequence having 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequence set forth in SEQ ID NO:20.

23. An expression vector comprising the nucleic acid of any one of claims 22-23.

24. An isolated host cell comprising the expression vector of claim 23.

25. A method of detecting AXL, the method comprising:
a) contacting a sample with the isolated antibody or antigen binding fragment thereof of any one of claim 1-5 or 13-17, under conditions to bind said antibody to an AXL on said sample, wherein the binding generates the production of a receptor/antibody complex; and
b) detecting the presence of the receptor/antibody complexes,
wherein the detecting comprises the presence or absence of the AXL on said sample.

26. A method of treating or preventing a disease or disorder associated with AXL in a subject, the method comprising:
a) contacting a sample known or suspected to contain AXL with the isolated antibody or antigen binding fragment thereof of any one of claim 1-5 or 13-17;
b) detecting the presence of complexes comprising AXL and the antibody; wherein the presence of the complexes indicates the presence of a disease or disorder; and,
c) administering to the subject the isolated antibody or antigen binding fragment thereof of any one of claim 1-5 or 13-17.

27. A method of diagnosing a disease or disorder, the method comprising:
a) isolating a sample from a subject;
b) incubating the sample with the isolated antibody or antigen binding fragment thereof of any one of claim 1-5 or 13-17, for a period of time sufficient to generate AXL:anti-AXL complexes;
c) detecting the presence or absence of the AXL:anti-AXL complexes from the isolated tissue; and,
d) associating presence or abundance of AXL with a location of interest of a tissue sample.


SEQUENCE LISTING (63 sequences)

SEQ ID NO: 1 - protein, 118 aa, synthetic construct
DVQLQESGPDLVKPSQSLSLTCTVTGYSITSAYSWHWIRQFPGNKLEWMGYIHYSGSTNYNPSLKSRISITRDTSKNQFFLQLNSVTTEDTATYYCARSLWFYAMDYWGQGTSVTVSS

SEQ ID NO: 2 - protein, 118 aa, synthetic construct
QIQLVQSGPELKKPGETVKISCKASGYTFTNYGMNWVKQAPGKGLKWMGWINTYIGEPTYADDFKGRFAFSLETSASTAYLQINNLKNEDMGTYFCARGTTFYAMDYWGQGTSVTVSS

SEQ ID NO: 3 - protein, 118 aa, synthetic construct
DVQLQESGPDLVKPSQSLSLTCTVTGYSITSAYSWHWIRQFPGNKLEWMGYIHYSGSTNYNPSLKSRISITRDTSKNQFFLQLSSVTTEDTATYYCARSLWFYAMDYWGQGTSVTVSS

SEQ ID NO: 4 - protein, 118 aa, synthetic construct
QIQLVQSGPELKKPGETVKISCKASGYTFTNYGMNWVKQAPGKGLKWMGWINTYTGEPTYADDFKGRFAFSLETSASTAYLQINNLKNEDMATYFCARGDRFYAMDYWGQGTSVTVSS

SEQ ID NO: 5 - protein, 117 aa, synthetic construct
EVNLVESGGGLVQPGGSLKLSCATSGFTFSDYYMYWVRQTPEKRLEWVAYISNGGGSTYYPDTVKGRFTISRDNAKNTLYLQMSRLKSEDTAMYYCARLHFYAMDYWGQGTSVTVSS

SEQ ID NO: 6 - protein, 107 aa, synthetic construct
DVQITQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIK

SEQ ID NO: 7 - protein, 107 aa, synthetic construct
DIQMNQSPSSLSASLGDTITITCHASQNINVWLNWYQQKPGNIPKLLIYKASNLHTGVPSRFSGSGSGTGFTLTISSLQPEDIATYYCQQGQSYPLTFGGGTKLEIK

SEQ ID NO: 8 - protein, 107 aa, synthetic construct
EIVLTQSPTTMAASPGEKITITCSASSSISSNYLHWYQQKPGFSPKLLIYRTSNLASGVPARFSGSGSGTSYSLTIGTMEAEDVATYYCQQGSSILTFGAGTKLELK

SEQ ID NO: 9 - protein, 107 aa, synthetic construct
DVQITQSPSYLAASPGETITIKCRASKSISKYLAWYQEKPGKTNKLLIYSGSTLHSGIPSRFSGSGSGTDFTLTISSLEPEDFAMYYCQQHNEYPWTFGGGTKLEIK

SEQ ID NO: 10 - protein, 107 aa, synthetic construct
EIVLTQSPTTMAASPGEKITITCSASSSISSNYLHWYQQKPGFSPKLLIYRTSNLASGVPTRFSGSGSGTSYSLTIGTMEAEDVATYYCQQGSSIFTFGSGTKLEIK

SEQ ID NO: 11 - protein, 107 aa, synthetic construct
DIVMTQSHKFMSTSVGDRVSITCKASQDVSTAVAWYQQKPGQSPKLLIYWASTRHTGVPDRFTGSGSGTDYTLTISSVQAEDLALYYCQQHYNTPFTFGSGTKLEIK

SEQ ID NO: 12 - DNA, 408 bp, synthetic construct
  source 1..408: mol_type = other DNA
atgagagtgctgattcttttgtgcctgttcacagcctttcctggtatcctgtctgatgtgcagcttcaggagtcaggacctgacctggtgaaaccttctcagtcactttcactcacctgcactgtcactggctactccatcaccagtgcttatagctggcactggatccggcagtttccaggaaacaaactggaatggatgggctacatacactacagtggtagcactaactacaacccatctctcaaaagtcgaatctctatcactcgagacacatccaagaaccagttcttcctgcagttgaattctgtgactactgaggacacagccacatattactgtgcaagaagcctatggttctatgctatggactactggggtcaaggaacctcagtcaccgtctcctca

SEQ ID NO: 13 - DNA, 408 bp, synthetic construct; identical to SEQ ID NO: 12
  source 1..408: mol_type = other DNA

SEQ ID NO: 14 - DNA, 411 bp, synthetic construct
  source 1..411: mol_type = other DNA
atggattggctgtggaacttgctattcctgatggcagctgcccaaagtgcccaagcacagatccagttggtgcagtctggacctgagctgaagaagcctggagagacagtcaagatctcctgcaaggcttctggatataccttcacaaactatggaatgaactgggtgaagcaggctccaggaaagggtttgaagtggatgggctggataaacacctacattggagagccaacatatgctgatgacttcaagggacggtttgccttctctttggaaacctctgccagcactgcctatttgcagatcaacaacctcaaaaatgaggacatgggtacatatttctgtgcaagggggactacgttctatgctatggactactggggtcaaggaacctcagtcaccgtctcctca

SEQ ID NO: 15 - DNA, 408 bp, synthetic construct
  source 1..408: mol_type = other DNA
atgagagtgctgattcttttgtgcctgttcacagcctttcctggtatcctgtctgatgtgcagcttcaggagtcaggacctgacctggtgaaaccttctcagtcactttcactcacctgcactgtcactggctactccatcaccagtgcttatagctggcactggatccggcagtttccaggaaacaaactggaatggatgggctacatacactacagtggtagcactaactacaacccatctctcaaaagtcgaatctctatcactcgagacacatccaagaaccagttcttcctgcagttgagttctgtgactactgaggacacagccacatattactgtgcaagaagcctatggttctatgctatggactactggggtcaaggaacctcagtcaccgtctcctca

SEQ ID NO: 16 - DNA, 411 bp, synthetic construct
  source 1..411: mol_type = other DNA
atggattggctgtggaacttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacatggctacatatttctgtgcaagaggtgataggttctatgctatggactactggggtcaaggaacctcagtcaccgtctcctca

SEQ ID NO: 17 - DNA, 408 bp, synthetic construct
  source 1..408: mol_type = other DNA
atgaacttggggctcagcttgattttccttgtccttgttttaaaaggtgtccagtgtgaagtgaacctggtggagtctgggggaggcttagtgcagcctggagggtccctgaaactctcctgtgcaacctctggattcactttcagtgactattacatgtattgggttcgccagactccagagaagaggctggagtgggtcgcatacattagtaatggtggtggtagcacctattatccagacactgtaaagggccgattcaccatctccagagacaatgccaagaacaccctgtacctgcaaatgagccgtctgaagtctgaggacacagccatgtattactgtgcaagacttcatttctatgctatggactactggggtcaaggaacctcagtcaccgtctcctca

SEQ ID NO: 18 - DNA, 381 bp, synthetic construct
  source 1..381: mol_type = other DNA
atgaggttccaggttcaggttctggggctccttctgctctggatatcaggtgcccagtgtgatgtccagataacccagtctccatcttatcttgctgcatctcctggagaaaccattactattaattgcagggcaagtaagagcattagcaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacagcataatgaatacccgtggacgttcggtggaggcaccaagctggaaatcaaa

SEQ ID NO: 19 - DNA, 381 bp, synthetic construct
  source 1..381: mol_type = other DNA
atgagggtccttgctgagctcctggggctgctgctgttctgctttttaggtgtgagatgtgacatccagatgaaccagtctccatccagtctgtctgcatcccttggagacacaattaccatcacttgccatgccagtcagaacattaatgtttggttaaactggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatcctctgacgttcggtggaggcaccaagctggaaatcaaa

SEQ ID NO: 20 - DNA, 375 bp, synthetic construct
  source 1..375: mol_type = other DNA
atgcagattatcagcttgctgctaatcagtgtcacagtcatagtgtctaatggagaaattgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtatactcacgttcggtgctgggaccaagctggagctgaaa

SEQ ID NO: 21 - DNA, 381 bp, synthetic construct
  source 1..381: mol_type = other DNA
atgaggttccaggttcaggttctggggctccttctgctctggatatcaggtgcccagtgtgatgtccagataacccagtctccatcctatcttgctgcatctcctggagaaaccattactattaaatgcagggcaagtaagagcattagtaaatatttagcctggtatcaagagaaacctgggaaaactaataagcttcttatctactctggatccactttgcattctggaattccatcaaggttcagtggcagtggatctggtacagatttcactctcaccatcagtagcctggagcctgaagattttgcaatgtattactgtcaacagcataatgaatacccgtggacgttcggtggaggcaccaagctggaaatcaaa

SEQ ID NO: 22 - DNA, 375 bp, synthetic construct
  source 1..375: mol_type = other DNA
atgcagattatcagcttgctgctaatcagtgtcacagtcatagtgtctaatggagaaattgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccaactcgcttcagtggcagtgggtctgggacctcttactctctcacaattggcaccatggaggctgaagatgttgccacttactactgccagcagggtagtagtatattcacgttcggctcggggacaaagttggaaataaaa

SEQ ID NO: 23 - DNA, 381 bp, synthetic construct
  source 1..381: mol_type = other DNA
atggagtcacagattcaggcatttgtattcgtgtttctctggttgtctggtgttgacggagacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattataacactccattcacgttcggctcggggacaaagttggaaataaaa

SEQ ID NO: 24 - protein, 6 aa, synthetic construct
SAYSWH

SEQ ID NO: 25 - protein, 5 aa, synthetic construct
NYGMN

SEQ ID NO: 26 - protein, 5 aa, synthetic construct
DYYMY

SEQ ID NO: 27 - protein, 16 aa, synthetic construct
YIHYSGSTNYNPSLKS

SEQ ID NO: 28 - protein, 17 aa, synthetic construct
WINTYIGEPTYADDFKG

SEQ ID NO: 29 - protein, 17 aa, synthetic construct
WINTYTGEPTYADDFKG

SEQ ID NO: 30 - protein, 17 aa, synthetic construct
YISNGGGSTYYPDTVKG

SEQ ID NO: 31 - protein, 9 aa, synthetic construct
SLWFYAMDY

SEQ ID NO: 32 - protein, 9 aa, synthetic construct
GTTFYAMDY

SEQ ID NO: 33 - protein, 9 aa, synthetic construct
GDRFYAMDY

SEQ ID NO: 34 - protein, 8 aa, synthetic construct
LHFYAMDY

SEQ ID NO: 35 - protein, 11 aa, synthetic construct
RASKSISKYLA

SEQ ID NO: 36 - protein, 11 aa, synthetic construct
HASQNINVWLN

SEQ ID NO: 37 - protein, 12 aa, synthetic construct
SASSSISSNYLH

SEQ ID NO: 38 - protein, 11 aa, synthetic construct
KASQDVSTAVA

SEQ ID NO: 39 - protein, 7 aa, synthetic construct
SGSTLQS

SEQ ID NO: 40 - protein, 7 aa, synthetic construct
KASNLHT

SEQ ID NO: 41 - protein, 7 aa, synthetic construct
RTSNLAS

SEQ ID NO: 42 - protein, 7 aa, synthetic construct
SGSTLHS

SEQ ID NO: 43 - protein, 7 aa, synthetic construct
WASTRHT

SEQ ID NO: 44 - protein, 9 aa, synthetic construct
QQHNEYPWT

SEQ ID NO: 45 - protein, 9 aa, synthetic construct
QQGQSYPLT

SEQ ID NO: 46 - protein, 8 aa, synthetic construct
QQGSSILT

SEQ ID NO: 47 - protein, 8 aa, synthetic construct
QQGSSIFT

SEQ ID NO: 48 - protein, 9 aa, synthetic construct
QQHYNTPFT

SEQ ID NO: 49 - protein, 18 aa, synthetic construct
MRVLILLCLFTAFPGILS

SEQ ID NO: 50 - protein, 19 aa, synthetic construct
MDWLWNLLFLMAAAQSAQA

SEQ ID NO: 51 - protein, 19 aa, synthetic construct
MNLGLSLIFLVLVLKGVQC

SEQ ID NO: 52 - protein, 20 aa, synthetic construct
MRFQVQVLGLLLLWISGAQC

SEQ ID NO: 53 - protein, 20 aa, synthetic construct
MRVLAELLGLLLFCFLGVRC

SEQ ID NO: 54 - protein, 18 aa, synthetic construct
MQIISLLLISVTVIVSNG

SEQ ID NO: 55 - protein, 20 aa, synthetic construct
MESQIQAFVFVFLWLSGVDG

SEQ ID NO: 56 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 57-58 - skipped (2 entries)

SEQ ID NO: 59 - protein, 9 aa, synthetic construct
  VARIANT 1: note = X1 is S, G or no amino acid
  VARIANT 2: note = X2 is L, T or D
  VARIANT 3: note = X3 is W, T, R or H
XXXFYAMDY

SEQ ID NO: 60-62 - skipped (3 entries)

SEQ ID NO: 63 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK